(19) United States
(12) Patent Application Publication
(10) Pub. No.: US 2023/0194530 A1
(43) Pub. Date: Jun. 22, 2023

(54) CIRCULATING CARDIOVASCULAR BIOMARKERS AND VASCULAR STABILIZING THERAPY

(71) Applicant: UNIVERSITY HEALTH NETWORK, Toronto (CA)

(72) Inventors: Kathryn HOWE, Toronto (CA); Jason FISH, Toronto (CA); Dakota GUSTAFSON, Toronto (CA); Paaladinesh THAVENDIRANATHAN, Toronto (CA); Katherin HANNEMAN, Toronto (CA); Kevin KAIN, Toronto (CA)

(21) Appl. No.: 18/067,318

(22) Filed: Dec. 16, 2022

Related U.S. Application Data
(60) Provisional application No. 63/290,560, filed on Dec. 16, 2021.

(51) Int. Cl. G01N 33/569; C12Q 1/70

(52) U.S. Cl. CPC G01N 33/56983; C12Q 1/701; C12Q 2600/16; C12Q 2600/178; C12Q 2600/158; C12Q 1/6869

(57) ABSTRACT

Recent literature on SARS-CoV-2 pathogenesis has suggested that the induction of substantial acute respiratory distress phenotypes is driven by a mismatched inflammatory response together with broad vascular dysfunction. While several detailed reports implementing multi-omic approaches have provided insight into the immune cell phenotypes involved in these processes, risk stratifying markers specific to COVID-19 and the vasculature have not been explored. Provided herein is a comprehensive, multi-omics-based description of the molecular antecedents to COVID-19 mortality, yielding new insights pertaining to the vasculature while highlighting the urgent need for clinical translation of novel biomarkers for disease prognosis.

### CROSS-REFERENCE TO RELATED APPLICATIONS

This application claims the benefit of U.S. Provisional Application No. 63/290,560, filed Dec. 16, 2021 and having same title, the entire content of which is incorporated herein by reference.

### TECHNICAL FIELD

This disclosure generally relate to the field of COVID-19 diagnostic risk assessment, and in particular diagnostic methods using biomarker panels, as well as treatments of Covid-19.

### BACKGROUND OF THE ART

Severe acute respiratory syndrome coronavirus 2 (SARS-CoV-2) is a highly contagious betacoronavirus, which results in coronavirus disease 2019 (COVID-19). While the majority of infected individuals manifest mild to moderate illness, 14-31% of symptomatic unvaccinated patients eventually require hospitalization, with intensive care unit (ICU) admission rates ranging from 2-26% among those hospitalized. Select populations, particularly older individuals and those with underlying comorbidities (including cardiovascular disease [CVD]) have high rates of morbidity and mortality. Substantially higher rates of morbidity and mortality have been observed as variants of concern become more predominant. While diagnostic testing has allowed for the rapid identification of COVID-19 cases, the lack of post-diagnosis risk assessment metrics, especially among the highest-risk subgroups, have undermined the cascade and allocation of care.

### SUMMARY

The use of existing cardiovascular and respiratory parameters could serve as a metric of risk prediction. However, case-fatality rates of those with comorbidities remain particularly high (e.g., preexisting CVD at ˜10.5%) with cardiorespiratory the aforementioned markers may have limited utility. In this regard, while standard metrics including measures of cardiac damage (e.g., troponin values above the 99^{th} percentile), the extent of inflammatory activation (e.g., C-reactive protein expression), and cardiovascular imaging have elucidated the spectrum of COVID-19 complications, they may have only modestly elucidated the risk of adverse in-hospital outcomes and may often provide limited insight into disease mechanism. Albeit encouraging, some COVID-19 therapeutics focus on stemming aberrant immune responses and controlling viral reproduction (e.g., tocilizumab and remdesevir, respectively), which may neglect key elements of the host response contributing to outcomes. From this perspective, clinical data and autopsy studies revealing endotheliitis and thrombosis have raised the possibility that endothelial dysfunction, particularly fluctuations in vascular integrity and coagulative capacity, could be a driver of clinical outcomes. Thrombosis, fluid extravasation, and microangiopathy observed in the small vessels and capillaries of the lungs may suggest that an intense vascular reaction takes place in those with severe disease.

Approaches suggest that viral tropism towards angiotensin-converting enzyme 2 and acetylated sialic acid residues, which are highly expressed by vascular endothelial cells (EC), could instigate cardiovascular dysfunction. Other approaches have alternatively suggested that SARS-CoV-2 may have limited infectious potential and replicative ability in ECs. In this respect, mechanisms secondary to direct infection, such as cascading immunological activation may instead be the driving factor behind the observed endothelial dysfunction; particularly among the populations with coexisting conditions where EC dysfunction is already evident. In fact, select endothelium-related biomarkers such as thrombomodulin, von Willebrand Factor, angiopoietin-2 (Ang-2), and soluble triggering receptor expressed on myeloid cells-1 (sTREM-1) may show utility in prognostication, being associated with both disease severity and in-hospital mortality. While markers of endothelial function may aid in prognostication, it seems possible that a simple combination of markers can provide insight significant enough to adjudicate the level of care a patient will need, nor is it apparent that these markers associate specifically with COVID-19 pathology. To date, the integration of clinical data with broad omics technologies has opened up new avenues for efficiently delineating complex patient phenotypes and their associations with clinical outcomes. Circulating profiles of plasma microRNAs (miRNA), in particular, have been shown to be tightly associated with disease, and capable of providing not only detailed prognostic information but also mechanistic insight.

The following seeks to characterize markers of endothelial function and inflammation and to develop for the first time an atlas of miRNA expression across both a spectrum of individuals diagnosed with COVID-19 as well as SARS-CoV-2-negative patients from the ICU. To enhance clinical utility, the following focuses on understanding how these markers are associated with in-hospital mortality of high-risk patients, particularly those requiring the highest levels of care.

In accordance with an aspect, there is provided a method of determining Coronavirus Disease 2019 (COVID-19) severity in a subject. The method including obtaining a circulating blood sample from the subject, and obtaining a biomarker panel comprising one or more of angiopoietin-2 (Ang-2), endothelin-1 (ET-1), soluble intercellular adhesion molecule (sICAM), soluble vascular cell adhesion molecule (sVCAM), soluble E-selectin (sE-selectin), triggering receptor expressed on myeloid cells-1 (sTREM-1), interkeulin-6 (IL-6), interleukin-8 (IL-8), myeloperoxidase (MPO), and high-sensitivity cardiac troponin (hs-cTnI). A different level of at least one biomarker in the biomarker panel compared to a reference panel from a SARS-CoV-2-negative subject is indicative of COVID-19 severity.

In accordance with a further aspect, the biomarker panel may include Ang-2.

In accordance with a further aspect, the biomarker panel may include Ang-2, and MPO.

In accordance with a further aspect, the biomarker panel may include Ang-2, ET-1, sICAM-1, sVCAM-1, sE-Selectin, sTREM-1, IL-6, IL-8, MPO and hs-cTnI.

In accordance with a further aspect, an elevated level of at least one biomarker may be indicative of COVID-19 severity.

In accordance with an aspect, there is provided a method of determining mortality risk of a subject infected with SARS-CoV-2. The method including detecting in a circulating blood sample from the subject one or more circulating microRNA (miR) biomarkers selected from the group consisting of: one or more miR from miR-30 family, miR-181a-5p, miR-199a-3p, miR-4793-5p, miR-6080, and miR-6750-5p.

In accordance with a further aspect, the one or more biomarkers may be selected from the group consisting of: miR-30b, miR-30c, miR-30e, miR-181a-5p, miR-199a-3p, and miR-6080.

In accordance with a further aspect, the method may include amplifying and detecting a target miR in the blood sample using polymerase chain reaction (PCR).

In accordance with a further aspect, the method may include capturing and sequencing the detected miR.

In accordance with a further aspect, the method may include sequencing the detected miR using next-generation sequencing. In some embodiments, the next-generation sequencing comprises Illumina™, Roche 454™, or Ion Torrent Sequencing™.

In accordance with a further aspect, sequencing the detected miR may include a nuclease protection assay. Exemplary sequencing technique for miRNA includes nuclease protection assays that are coupled to sequencing, such as miRNA whole transcriptome assays (https://www.htgmolecular.com/assays/mirna-wta, the entire content of which is incorporated herein by reference).

In accordance with a further aspect, the method further including measuring the expression level of at least one of the one or more circulating miR biomarkers.

In accordance with a further aspect, the method may include measuring the expression level of two or more of the circulating miR biomarkers.

In accordance with a further aspect, the method may include measuring the miR expression level using a multiplex assay.

In accordance with a further aspect, the multiplex assay may be a 5-plex assay.

In accordance with a further aspect, detecting the one or more circulating miR biomarkers may include detecting miR using a colorimetic or a CRISPR-based biosensor. Exemplary CRISPR-based biosensor includes CRISPR/Cas13a-powered electrochemical microfluidic biosensors (https://onlinelibrary.wiley.com/doi/full/10.1002/adma.201905311, the entire content of which is incorporated herein by reference).

In accordance with an aspect, there is provided a kit including one or more antibodies specific to angiopoietin-2 (Ang-2), endothelin-1 (ET-1), soluble intercellular adhesion molecule (sICAM), soluble vascular cell adhesion molecule (sVCAM), soluble E-selectin (sE-selectin), triggering receptor expressed on myeloid cells-1 (sTREM-1), interkeulin-6 (IL-6), IL-8, myeloperoxidase (MPO), or high-sensitivity cardiac troponin (hs-cTnI).

In accordance with a further aspect, the kit may include an antibody specific to Ang-2.

In accordance with a further aspect, the kit may include a first antibody specific to Ang-2, and a second antibody specific to MPO.

In accordance with a further aspect, the kit may further include one or more antibodies specific to a biomarker selected from ET-1, sICAM-1, sVCAM-1, sE-Selectin, sTREM-1, IL-6, IL-8, and hs-cTnI.

In accordance with an aspect, there is provided a kit including one or more probes for binding a circulating microRNA (miR) biomarker selected from the group consisting of one or more miR from miR-30 family, miR-181a-5p, miR-199a-3p, miR-4793-5p, miR-6080, and miR-6750-5p.

In accordance with a further aspect, the biomarker may be selected from the group consisting of miR-30b, miR-30c, miR-30e, miR-181a-5p, miR-199a-3p, and miR-6080.

In accordance with a further aspect, the kit may be for an assay.

In accordance with a further aspect, the one or more probes may be amplification probes.

In accordance with a further aspect, the one or more probes may be sequencing probes.

In accordance with an aspect, there is provided a method of treating Coronavirus Disease 2019 (COVID-19) in a subject or inhibiting the development of COVID-19 or a sequelae resulting from COVID-19 in a subject. The method including administering said subject with Slit2, or a variant or fragment thereof.

In accordance with a further aspect, the method may include administering the subject with a full-length Slit2.

In accordance with a further aspect, the method may include administering the subject with a N-terminal domain of Slit2 (Slit2-N).

In accordance with an aspect, there is provided a method of treating Coronavirus Disease 2019 (COVID-19) in a subject or inhibiting the development of COVID-19 or a sequelae resulting from COVID-19 in a subject. The method including upregulating the Slit2 signaling pathway in the subject.

In accordance with a further aspect, the method may include administering the subject with one or more factors or polypeptides from the Slit2 signaling pathway.

In accordance with a further aspect, the one or more factors or polypeptides from the Slit2 signaling pathway may be GIT1, ARF6, Ab1, GTPase, Ena, Myo9b, or Hakai.

In accordance with an aspect there is provided a use of Slit2 or a variant or fragment thereof in the treatment or inhibition of the development of Coronavirus Disease 2019 (COVID-19) or a sequelae resulting from COVID-19 in a subject.

In accordance with an aspect there is provided a use of Slit2 or a variant or fragment thereof in the manufacture of a medicament for the treatment or inhibition of the development of Coronavirus Disease 2019 (COVID-19) or a sequelae resulting from COVID-19 in a subject.

In accordance with a further aspect, the use may include use of a full-length Slit2.

In accordance with a further aspect, the use may include use of a N-terminal domain of Slit2 (Slit2-N).

In accordance with an aspect there is provided use of one or more factors or polypeptides from the Slit2 signaling pathway for the treatment of Coronavirus Disease 2019 (COVID-19) in a subject or inhibiting the development of COVID-19 or a sequelae resulting from COVID-19 in a subject by upregulating the Slit2 signaling pathway in the subject.

In accordance with an aspect there is provided use of one or more factors or polypeptides from the Slit2 signaling pathway for the manufacture of a medicament for the treatment of Coronavirus Disease 2019 (COVID-19) in a subject or inhibiting the development of COVID-19 or a sequelae resulting from COVID-19 in a subject.

### DESCRIPTION OF THE DRAWINGS

FIG. 1: Unadjusted Kaplan-Meier Estimates of Survival, according to some embodiments.

FIG. 2: Plasma Concentration of Endothelial Dysfunction and Inflammatory Markers at T₀₋₁, according to some embodiments.

FIG. 3: Plasma MiRNA Transcriptome Across the COVID-19 Severity, according to some embodiments.

FIG. 4: Machine Learning Approach to Risk Assessment and Association of Biomarkers with In-Hospital Mortality for Severe COVID-19 Patients, according to some embodiments.

FIG. 5: Endothelial Barrier Disruption Driven by Modulation of Inflammatory and Cytoskeletal Pathways is Disease Severity Dependent, according to some embodiments.

FIG. 6: Targeted Modulators of Endothelial Barrier Protect Against COVID-19 Plasma-Induced Endothelial Barrier Dysfunction, according to some embodiments.

FIG. 7: Flow diagram of patients enrolled between the COLOBILI Study (St. Michael's Hospital) and the COVID Study (University Health Network), according to some embodiments.

FIG. 8: Spike (trimer) antigen serology testing from patients having a negative SARS-CoV-2 polymerase chain reaction result, according to some embodiments.

FIG. 9: The association of coronary artery disease with mortality characterized in terms of proportion of deceased patients stratified by status, according to some embodiments.

FIG. 10: Spearman correlations between t₀₋₁ concentrations of biomarkers amongst the entire cohort (SARS-CoV-2 negative and positive populations), according to some embodiments. Values presented in the graph correspond to the graphic representations.

FIG. 11: Spearman correlations between t₀₋₁ concentrations of biomarkers within the mild COVID-19 subgroup, according to some embodiments. Values presented in the graph correspond to the graphic representations.

FIG. 12: Spearman correlations between t₀₋₁ concentrations of biomarkers within the severe COVID-19 subgroup, according to some embodiments. Values presented in the graph correspond to the graphic representations.

FIG. 13: Spearman correlations between t₀₋₁ concentrations of biomarkers within the COVID-19 subgroup, according to some embodiments. Values presented in the graph correspond to the graphic representations.

FIG. 14: Spearman correlations between t₀₋₁ concentrations of biomarkers within the mild SARS-CoV-2 negative subgroup, according to some embodiments. Values presented in the graph correspond to the graphic representations.

FIG. 15: Spearman correlations between t₀₋₁ concentrations of biomarkers within the severe SARS-CoV-2 negative subgroup, according to some embodiments. Values presented in the graph correspond to the graphic representations.

FIG. 16. Plasma Concentration of Endothelial Dysfunction and Inflammatory Markers at t₀₋₁. (a) ET-1, (b) sICAM-1, (c) sE-Selectin, (d) sTREM-1, (e) IL-6, (f) IL-8, (g) MPO, and (h) hs-cTnI stratified among disease severity, according to some embodiments.

FIG. 17: Plasma Concentration of Endothelial Dysfunction and Inflammatory Markers at t₀₋₁ and ability to discriminate survival in ICU patients, according to some embodiments. Severe COVID-19 patients and severe negative patients (i.e., SARS-CoV-2 negative) (a) Levels of Angiopoietin-2, (b) Endothelin-1, (c) sICAM-1, (d) sVCAM-1, (e) sE-Selectin, (f) sTREM-1, (g) IL-6, (h) IL-8, (i) MPO, and (j) hs-cTnI stratified among disease severity.

FIG. 18: Plasma Concentration of Endothelial Dysfunction and Immunological Markers at t₀₋₁ in ICU patients, according to some embodiments. (a) Levels of Angiopoietin-2, (b) Endothelin-1, (c)sICAM-1, (d) sVCAM-1, (e) sE-Selectin, (f) sTREM-1, (g) IL-6, (h) IL-8, (i) MPO, and (j) hs-cTnI stratified among disease severity. For each pair of clatter plots, severe COVID-19 is presented on the left while severe SARS-CoV-2 negative is presented on the right.

FIG. 19: Plasma Concentration of (a) IL-6 and (b) MPO, longitudinally between severe COVID-19 patients and severe SARS-CoV-2 negative patients, according to some embodiments.

FIG. 20: Plasma MicroRNA Transcriptome Across the Disease Severity Subgroups. Volcano plots of differentially expressed miRNA between patient groups (a, c, e) with predicted KEGG terms (with enrichment score below and number of genes to the right) for pathways of deregulated microRNAs shown beside each corresponding region of the volcano plot (b, d, f), according to some embodiments.

FIG. 21: Plasma MicroRNA Transcriptome Across the Disease Severity Subgroups. Volcano plots of differentially expressed miRNA between patient groups (a, c, e) with predicted KEGG terms (with enrichment score below and number of genes to the right) for pathways of deregulated microRNAs shown beside each corresponding region of the volcano plot (b, d, f), according to some embodiments.

FIG. 22: Feature importance of a machine learning model incorporating clinical data, according to some embodiments.

FIG. 23: Feature importance of a machine learning model incorporating both clinical data and protein expression metrics, according to some embodiments.

FIG. 24; Association of Biomarkers with In-Hospital Mortality for Severe COVID-19 Patients, according to some embodiments. Univariable log hazard ratios of candidate microRNAs (a) hsa-miR-181a-5p, (b) hsa-miR-199a-3p, and c) hsa-miR-339-3p.

FIG. 25: T₀₋₁ COVID-19 Patient Plasma Selectively Induces Acute Increases in Endothelial Permeability, according to some embodiments. (a) Permeability of pHUVEC monolayers was measured by 40 kDa FITC extravasation from the apical to the basolateral surface one-hour post-co-incubation. Treatment groups were normalized to the negative control. (b) Permeability of pHUVEC monolayers was measured by 40 kDa FITC extravasation from the apical to the basolateral surface six hours post-co-incubation.

FIG. 26: Correlation of t₀₋₁ Plasma Cardiovascular Biomarkers in COVID-19 positive patients to Induction of Endothelial Permeability, according to some embodiments. Pearson correlations between (a) hemolysis, (b) Ang-2, (c) hs-cTnI, (d) sE-Selectin, (e) ET-1, (f) sICAM-1, (g) IL-6, (h) IL-8, (i) sTREM-1, (j) sVCAM-1, and (k) MPO to the change in pHUVEC TEER after six-hours co-incubation; n=111 per correlation.

FIG. 27: Endogenous sSlit2 is upregulated in severe COVID-19 patient plasma, according to some embodiments. (a) Endogenous sSlit2 at t₀₋₁ across the severity of COVID-19 (n=27-40, severe vs negative, *P=0.0279). (b) Endogenous sSlit2 at longitudinal intervals in patients with severe COVID-19 (n=14-38).

### DETAILED DESCRIPTION

Markers of endothelial dysfunction at presentation, while indicative of poor outcomes in COVID-19-positive patients, reflect systemic vascular dysfunction in critically ill patients and are not specific to SARS-CoV-2 infection.

Generation of a plasma microRNA atlas uncovers COVID-19-specific prognostic markers and multiple disease-specific pathways of interest, including endothelial barrier dysfunction.

Synthesis of electronic health record data with clinically relevant multi-omic datasets using a machine learning approach provide substantially better metrics by which mortality can be estimated in patients with severe COVID-19.

Targeted stabilization of the endothelial barrier with Slit2-N present novel therapeutic avenues that should be explored in COVID-19 patients.

Endothelial cell (EC) activation, endotheliitis, vascular permeability, and thrombosis have been observed in patients with severe COVID-19, indicating that the vasculature may be affected during the acute stages of SARS-CoV-2 infection. Prior to the present disclosure, it was unknown whether circulating vascular markers can be sufficient to predict clinical outcomes, unique to COVID-19 related pathology, and if vascular permeability can be therapeutically targeted.

Evaluating the prevalence of circulating inflammatory, cardiac, EC activation, and the development of a microRNA atlas in 241 patients with suspected SARS-CoV-2 infection, allowed their prognostic value to be assessed by a Random Forest model machine learning approach. Subsequent ex vivo experiments were performed to assess EC permeability responses to patient plasma and derive modulated gene regulatory networks from which rational therapeutic design was inferred.

Multiple inflammatory and EC activation biomarkers were associated with mortality in COVID-19 patients and in severity-matched SARS-CoV-2-negative patients. In contrast, dysregulation of specific microRNAs at presentation was specific for poor COVID-19-related outcomes and revealed disease-relevant pathways. Integrating the datasets using a machine learning approach further enhanced clinical risk prediction for in-hospital mortality. Exposure of ECs to COVID-19 patient plasma resulted in severity-specific gene expression responses and EC barrier dysfunction which was ameliorated using angiopoietin-1 mimetic or recombinant Slit2-N.

Integration of multi-omics data reveal microRNA and vascular biomarkers prognostic of in-hospital mortality in COVID-19 patients and that vascular stabilizing therapies should be explored as a treatment for the endothelial dysfunction of COVID-19, and other severe diseases where endothelial dysfunction has a central role in pathogenesis.

The induction of substantial acute respiratory distress phenotypes may be driven by a mismatched inflammatory response together with broad vascular dysfunction. Approaches implementing multi-omic approaches have provided insight into the immune cell phenotypes involved in these processes, risk stratifying markers specific to COVID-19 and the vasculature have not been explored. A comprehensive, multi-omics-based description of the molecular antecedents to COVID-19 mortality is herein presented, yielding new insights pertaining to the vasculature while highlighting the urgent need for clinical translation of novel biomarkers for disease prognosis.

Severe acute respiratory syndrome coronavirus 2 (SARS-CoV-2) is a highly contagious betacoronavirus, which results in coronavirus disease 2019 (COVID-19).(1) While the majority of infected individuals manifest mild to moderate illness, 14-31% of symptomatic unvaccinated patients eventually require hospitalization, with intensive care unit (ICU) admission rates ranging from 2-26% among those hospitalized.(2) Select populations, particularly older individuals and those with underlying comorbidities (including cardiovascular disease [CVD]) have high rates of morbidity and mortality.(3) Substantially higher rates of morbidity and mortality have been observed as variants of concern become more predominant.(4) While diagnostic testing has allowed for the rapid identification of COVID-19 cases, the lack of post-diagnosis risk assessment metrics, especially among the highest-risk subgroups, have undermined the cascade and allocation of care.

The use of existing cardiovascular and respiratory parameters could serve as a metric of risk prediction.(5, 6) However, case-fatality rates of those with comorbidities remain particularly high (e.g., preexisting CVD at ˜10.5%) with cardiorespiratory the aforementioned markers may have limited utility.(7) In this regard, while standard metrics including measures of cardiac damage (e.g., troponin values above the 99^{th} percentile), the extent of inflammatory activation (e.g., C-reactive protein expression), and cardiovascular imaging have elucidated the spectrum of COVID-19 complications, they may have only modestly elucidated the risk of adverse in-hospital outcomes and may often provide limited insight into disease mechanism.(8-10) Albeit encouraging, some COVID-19 therapeutics focus on stemming aberrant immune responses and controlling viral reproduction (e.g., tocilizumab and remdesevir, respectively), which may neglect key elements of the host response contributing to outcomes.(11) From this perspective, clinical data and autopsy studies revealing endotheliitis and thrombosis have raised the possibility that endothelial dysfunction, particularly fluctuations in vascular integrity and coagulative capacity, could be a driver of clinical outcomes.(12) Thrombosis, fluid extravasation, and microangiopathy observed in the small vessels and capillaries of the lungs may suggest that an intense vascular reaction takes place in those with severe disease.(13)

Viral tropism towards angiotensin-converting enzyme 2 and acetylated sialic acid residues, which are highly expressed by vascular endothelial cells (EC), could instigate cardiovascular dysfunction.(14-19) However, SARS-CoV-2 may have limited infectious potential and replicative ability in ECs.(20) In this respect, mechanisms secondary to direct infection, such as cascading immunological activation may instead be the driving factor behind the observed endothelial dysfunction; particularly among the populations with coexisting conditions where EC dysfunction is already evident.(21, 22) In fact, select endothelium-related biomarkers such as thrombomodulin(23), von Willebrand Factor(24), angiopoietin-2 (Ang-2)(25), and soluble triggering receptor expressed on myeloid cells-1 (sTREM-1)(26) may show utility in prognostication, being associated with both disease severity and in-hospital mortality. While markers of endothelial function may aid in prognostication, it seems possible that a simple combination of markers can provide insight significant enough to adjudicate the level of care a patient will need, nor is it apparent that these markers associate specifically with COVID-19 pathology. To date, the integration of clinical data with broad omics technologies has opened up new avenues for efficiently delineating complex patient phenotypes and their associations with clinical outcomes.(27, 28) Circulating profiles of plasma microRNAs (miRNA), in particular, have been shown to be tightly associated with disease, and capable of providing not only detailed prognostic information but also mechanistic insight.(29, 30)

The following seeks to characterize markers of endothelial function and inflammation and to develop for the first time an atlas of miRNA expression across both a spectrum of individuals diagnosed with COVID-19 as well as SARS-CoV-2-negative patients from the ICU. To enhance clinical utility, the following focuses on understanding how these markers are associated with in-hospital mortality of high-risk patients, particularly those requiring the highest levels of care.

The present disclosure provides methods for determining Coronavirus Disease 2019 (COVID-19) severity. In some embodiments, methods for determining COVID-19 severity in a subject comprise obtaining a circulating blood sample from the subject; and obtaining a biomarker panel comprising one or more of angiopoietin-2 (Ang-2), endothelin-1 (ET-1), soluble intercellular adhesion molecule (sICAM), soluble vascular cell adhesion molecule (sVCAM), soluble E-selectin (sE-selectin), triggering receptor expressed on myeloid cells-1 (sTREM-1), interkeulin-6 (IL-6), interleukin-8 (IL-8), myeloperoxidase (MPO), and high-sensitivity cardiac troponin (hs-cTnI); wherein a different level of at least one biomarker in the biomarker panel compared to a reference panel from a SARS-CoV-2-negative subject is indicative of COVID-19 severity. In one embodiment, the biomarker panel comprises two or more of the biomarkers. In one embodiment, the biomarker panel comprises three or more of the biomarkers. In one embodiment, the biomarker panel comprises four or more of the biomarkers. In one embodiment, the biomarker panel comprises five or more of the biomarkers. In one embodiment, the biomarker panel comprises six or more of the biomarkers. In one embodiment, the biomarker panel comprises seven or more of the biomarkers. In one embodiment, the biomarker panel comprises eight or more of the biomarkers. In one embodiment, the biomarker panel comprises nine or more of the biomarkers. In one embodiment, the biomarker panel comprises all of the biomarkers.

In some embodiments, elevated levels of at least one biomarker is indicative of COVID-19 severity. In one embodiment, elevated levels of at least two biomarkers are indicative. In one embodiment, elevated levels of at least three biomarkers are indicative. In one embodiment, elevated levels of at least four biomarkers are indicative. In one embodiment, elevated levels of at least five biomarkers are indicative. In one embodiment, elevated levels of at least six biomarkers are indicative. In one embodiment, elevated levels of at least seven biomarkers are indicative. In one embodiment, elevated levels of at least eight biomarkers are indicative. In one embodiment, elevated levels of at least nine biomarkers are indicative. In one embodiment, elevated levels of all biomarkers are indicative.

In one embodiment, the biomarker panel comprises Ang-2, preferably Ang-2, and MPO. In one preferred embodiment, the biomarker panel comprises Ang-2, ET-1, sICAM-1, sVCAM-1, sE-Selectin, sTREM-1, IL-6, IL-8, MPO and hs-cTnI. Elevated levels of these biomarkers are indicative of COVID-19 severity.

In some embodiments, methods for determining mortality risk of a subject infected with SARS-CoV-2 comprise detecting in a circulating blood sample from the subject one or more circulating microRNA (miR) biomarkers selected from the group consisting of: one or more miR from miR-30 family, miR-181a-5p, miR-199a-3p, miR-4793-5p, miR-6080, and miR-6750-5p. In one embodiment, two or more of these miR biomarkers are detected. In one embodiment, three or more of these miR biomarkers are detected. In one embodiment, four or more of these miR biomarkers are detected. In one embodiment, five or more of these miR biomarkers are detected. In one embodiment, all of these miR biomarkers are detected. In one embodiment, the miR biomarkers are selected from miR-30b, miR-30c, miR-30e, miR-181a-5p, miR-199a-3p, and miR-6080.

In some embodiments, the circulating microRNA miR is amplified, detected, captured, and/or sequenced using polymerase chain reaction (PCR), CRISPR, colorimetry, next-generation sequencing (i.e. isothermal amplification, loop-mediated amplification, Illumina™, pyrosequencing, real-time sequencing, parallel sequencing, nanopore sequencing), and/or nucleotide assays (i.e. nuclease protection assay, multiplex assay, 5-plex multiplex assay). Various other amplified, detected, captured, and/or sequenced methods are available and applicable to the present methods.

Kits for ducting the methods and diagnoses disclosed herein are also provided, containing one or more of: one or more antibodies specific to the one or more biomarkers described herein, buffers, probes (amplification probes, sequencing probes, capture probes, and/or detection probes), assay reagents, and test tubes.

In some embodiments, subjects diagnosed or predicted to have COVID-19, severe COVID-19, moderate COVID-19, or mild COVID-19 are treated by administering said subject with Slit2, or a variant or fragment thereof. In some embodiments, subjects diagnosed for COVID-19 severity with the methods described herein are treated by administering said subject with Slit2, or a variant or fragment thereof. In some embodiments, methods are provided for treating COVID-19 in a subject or inhibiting the development of COVID-19 or a sequelae resulting from COVID-19 in a subject are provided comprising administering said subject with Slit2, or a variant or fragment thereof.

In one embodiment, a variant of Slit2 has at least 70%, at least 75%, at least 80%, at least 85%, at least 90%, at least 95% or at least 99% sequence identity with wild-type Slit2 protein, and has comparable biological activity. In one embodiment, a fragment of Slit2 comprise at least 50%, at least 60%, at least 70%, at least 80%, at least 90%, or at least 95% of a full length Slit2 protein. In one embodiment, the fragment is a N-terminal domain of Slit2.

In some embodiments, subjects diagnosed or predicted to have COVID-19, severe COVID-19, moderate COVID-19, or mild COVID-19 are treated by upregulating the Slit2 signaling pathway in the subject. In some embodiments, subjects diagnosed for COVID-19 severity with the methods described herein are treated by upregulating the Slit2 signaling pathway in the subject. In some embodiments, methods are provided for treating COVID-19 in a subject or inhibiting the development of COVID-19 or a sequelae resulting from COVID-19 in a subject are provided comprising upregulating the Slit2 signaling pathway in the subject.

In one embodiment, upregulating the Slit2 signaling pathway comprises administering said subject with one or more factors or polypeptides from the Slit2 signaling pathway from the following list: GIT1, ARF6, Ab1, GTPase, Ena, Myo9b, or Hakai. In one embodiment, two or more of these factors or polypeptides are administered. In one embodiment, three or more of these factors or polypeptides are administered. In one embodiment, four or more of these factors or polypeptides are administered. In one embodiment, five or more of these factors or polypeptides are administered. In one embodiment, six or more of these factors or polypeptides are administered. In one embodiment, all of these factors or polypeptides are administered.

EXAMPLES. The following is an exemplary, non-limiting embodiment of the ideas described herein.

Nonstandard Abbreviations and Acronyms

| | |
|---|---|
| Ang-2 | Angiopoietin-2 |
| ARDS | Acute respiratory distress syndrome |
| AUROC | Area under the receiver operating characteristic |
| BMI | Body mass index |
| COVID-19 | Coronavirus Disease 19 |
| CVD | Cardiovascular disease |
| EC | Endothelial cell |
| sE-Selectin | Soluble E-selectin |
| ET-1 | Endothelin-1 |
| FDR | False discovery rate |
| Hs-cTnI | High-sensitivity cardiac troponin |
| ICU | Intensive care unit |
| IL | Interleukin |
| IQR | Interquartile range |
| miR/miRNA | MicroRNA |
| MPO | Myeloperoxidase |
| S.D. | Standard deviation |
| SARS-COV-2 | Severe acute respiratory syndrome coronavirus 2 |
| sICAM-1 | Soluble intercellular adhesion molecule-1 |
| sTREM-1 | Soluble triggering receptor expressed on |
| | myeloid cells-1 |
| sVCAM-1 | Soluble vascular cell adhesion molecule-1 |
| VE-cadherin | Vascular endothelial-cadherin |

Methods

Reagent and Resource Sharing: Transcriptomic data (i.e., messenger RNA and miRNA sequencing) have been deposited at the Gene Expression Omnibus and the RStudio analysis pipeline is contained within the Supplement Section.

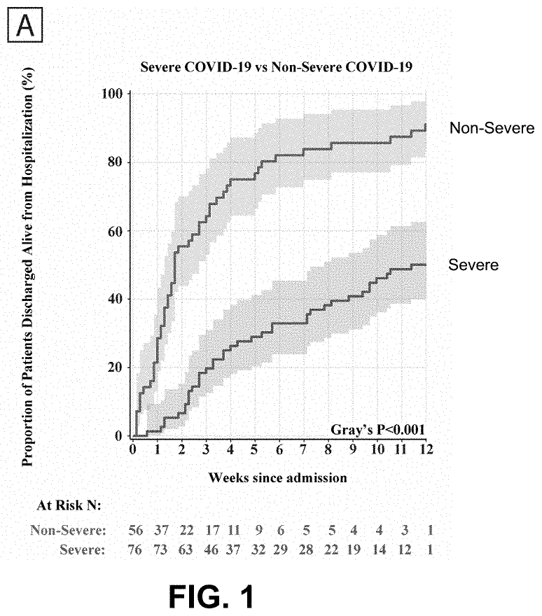

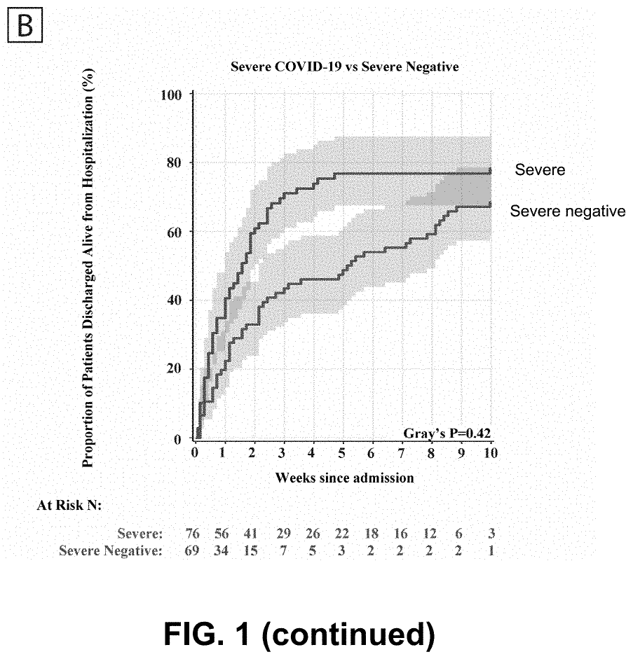

Synopsis of Study Design, Patient Demographics, and Clinical Severity: Two hundred and forty-one patients with suspected, community-acquired SARS-CoV-2 (acute infection) were enrolled prospectively in the emergency departments or upon admission at two urban, quaternary-care hospitals in Toronto, Canada (University Health Network and St. Michael's Hospital, Table 1 and FIG. 1) between May 2020 to December 2020 (prior to vaccine availability). Infection status of admitted patients was confirmed by at least two SARS-CoV-2 polymerase-chain reaction tests. Patients with SARS-CoV-2 but noninfectious etiologies were not enrolled (e.g., blunt force trauma). Medical history, physical examination, clinical laboratory values, and acute illness scores (Acute Physiologic Assessment and Chronic Health Evaluation II and Sequential Organ Failure Assessment) were recorded upon admission (day 0 or 1; t₀₋₁), two to three days later (t₂₋₃), and up to five days (t₄₋₅), along with the synchronous collection of blood samples (FIG. 1). As a result of sampling logistics, t₀ or t₁ (days) were grouped as the earliest timepoint available for admitted patients. Similarly, collapsed sampling timepoints at t₂₋₃ and t₄₋₅ allowed evaluation of patient trajectories. Since the analysis was conducted retrospectively, clinical care was dictated by individual care providers with the primary outcome being mortality.

The cohort of 241 patients was categorized into three groups that reflected conventional concepts of COVID-19 severity (National Institutes of Health, ‘Clinical Spectrum of SARS-CoV-2 Infection’), as well as two analogous symptom/severity matched control groups.(31) The resulting five groups were: SARS-CoV-2 negative patients presenting to outpatient clinics with symptoms consistent with a respiratory tract illness (n=30, “mild negative”); SARS-CoV-2 positive patients presenting to outpatient clinics with symptoms consistent with a respiratory tract illness (n=27, “mild COVID-19”); admitted SARS-CoV-2 positive patients requiring supplemental oxygenation (n=39, “moderate COVID-19”); SARS-CoV-2 positive patients requiring high-level in-patient ICU care (n=76, “severe COVID-19”); and SARS-CoV-2 negative patients who exhibited symptoms of a severe respiratory disease requiring high-level in-patient ICU care (n=69, “severe negative”). Patients testing negative by nasopharyngeal SARS-CoV-2 polymerase chain reaction had immunological history (i.e., antibody reactivity) ascertained through spike antigen cross-reactivity using a Federal Drug Administration approved enzyme-linked immunosorbent sandwich assay (FIG. II).

Data Visualization and Statistical Analysis: Descriptive Analysis—Clinical characteristics were characterized using summary statistics. Continuous variables were described using median and inter-quartile range (IQR), and dichotomous or polytomous variables were described using frequencies. Between-group differences were evaluated using Wilcoxon rank-sum tests for continuous variables and Fisher's exact tests for dichotomous/polytomous variables. Correlation between continuous variables were quantified using Spearman rank correlation. Descriptive outcome analysis—The Kaplan-Meier survival method was applied to assess the in-hospital death, and the between-group differences in the freedom from death were evaluated using log-rank tests. The length of hospitalization/ICU was characterized using competing risk models in terms of cumulative incidence rate function. Univariable Cox proportional hazard regression were applied to assess and quantify the association of the baseline clinical characteristics with in-hospital/ICU death. The associations of continuous variables were modeled using natural cubic splines. Biomarker Analysis—Comparisons between two independent groups were made using t-tests for normally distributed continuous variables or Wilcoxon rank-sum tests non-normally distributed continuous variables. When more than two groups were compared, either a one-way ANOVA with a Tukey or Bonferroni post-hoc test (where appropriate) for multiple testing correction, Kruskal-Wallis one-way analysis of variance with Dunn's multiple comparison correction. Two-way ANOVA was used to estimate how the mean quantitative variable changes according to time and group differences in leak experiments. Where appropriate, Benjamini-Hochberg false discovery rate (FDR) was utilized with adjusted P values (or Q value where stated) of <0.05 being considered statistically significant and indicated in the graphs as reported by the analysis software with significance thresholds of P<0.05, P<0.01, P<0.001, and P<0.0001 indicated as *, **, ***, **** respectively. MiRNA pathway analysis was conducted using BioCarta/KEGG/Reactome databases and tested for enrichment by a hypergeometric test with adjustment for multiple comparisons using the Benjamini-Hochberg FDR, with P ≤1.05 considered to be statistically enriched in a gene set of interest.(32-34) Although many hypotheses were tested throughout the manuscript, no experiment-wide multiple test correction was applied. Unless indicated otherwise, graphs depict averaged values of independent data points with technical replicates and have error bars displayed as mean +/−standard deviation (±S.D.). Data were analyzed with GraphPad Prism 9.0.0 for MacOS (GraphPad Software, Inc., La Jolla, Calif., USA; Biomarker Multiple Comparisons), R(35) (v4.0.3; Spearman Correlation Plots), and FIJI(36) (v2.1.0/1.53c; Quantifying Image Intensities). Final figures were assembled for publication purposes using Adobe Illustrator (v25.4.1).

Study Approval: This is a multicenter, secondary analysis of a prospectively recruited longitudinal cohort study enrolling consecutive patients with suspected SARS-CoV-2 infection who were referred to two Canadian quaternary care networks in Toronto, Canada from May 2020 to December 2020: University Health Network and St. Michael's Hospital. All participants who were 18 years of age or older, provided either direct written informed consent or were consented into the study by a lawfully entitled substitute decision-maker on behalf of a participant when lacking the capacity to make the decision. The study, and consenting, was conducted in accordance with protocols approved by the Research Ethics Board (REB) of the University Health Network (REB #: 20-5453.6; Cardiovascular Disease and Outcomes among Patients with SARS-CoV-2 Infection During Admission and Post-Discharge [The COVID study]) or St. Michael's Hospital (REB #: 20-078; COVID-19 Longitudinal Biomarkers in Lung Injury [COLOBILI]). SARS-CoV-2-negative patients with severe respiratory illness symptoms were enrolled within the COLOBILI study.

Detailed materials and methods can be found in the Supplement Section and the Major Resource Tables (Tables I and II).

Results

The baseline characteristics for the cohort are indicated in Table 1. The median age (interquartile range [IQR]) of the entire cohort (n=241) was 61 [51-72] years (155 male patients [64.3%]), and of those 201 were admitted, having a median hospital stay of 18 (IQR, 8-40) days within which 54 (26.9%) died (Table 1). Regardless of admission or SARS-CoV-2 status, co-existing medical conditions were common amongst the population including 83 (out of a total of 234; 35.5%) with diabetes mellitus, 80 (34.4%) with underlying CVD, 31 (12.9%) having chronic kidney disease, and 159 (66.0%) patients having more than one co-existing condition. In this respect, there were significant differences in age (P<0.0001), and the frequency of obesity (P=0.0006), smoking history (P<0.0001), chronic kidney disease (P=0.0204), diabetes mellitus (P<0.0001), dyslipidemia (P=0.0266), gastroesophageal reflux disease (P=0.0019), hypertension (P=0.0105), calcium channel blockers (P=0.0338), and diuretics (P=0.0088) between groups (Table 1). Although a small proportion of patients had documented smoking status, there were 40 (total 138; 29.0%) current or former smokers, with 33 (total 234; 14.1%) patients having chronic obstructive pulmonary disease.

On admission to hospital, clinical lab data highlighted disparities between the groups with leukocytosis and lymphopenia seen amongst more severe groups (e.g., white blood cell count [P<0.0001]; lymphocytes [P<0.0023, table 111]). Amongst cardiovascular markers, there were significant differences in the frequency of elevated creatine kinase (P<0.0001) and D-dimers (P<0.0001). Although the etiology of admissions was predominantly extracardiac in nature, high-sensitivity troponin 1 (hs-CnTl) values at t₀₋₁ were detectable (>3 pg/mL) in 157 patients (65.1%), with values significantly elevated (>15 pg/mL in males; >10 pg/mL in females) in 90 patients (37.3%, Table 111), predominantly in the severe groups (P<0.0001)

COVID-19 Outcomes During Hospital Admission: Among admitted patients with COVID-19, 33 died, with Kaplan-Meier survival curves indicating that patients with severe disease higher mortality risk than did patients with less severe phenotypes (P<0.001, FIG. 1A). Furthermore, the only baseline clinical metrics associated with mortality within admitted patients were the history of coronary artery disease (Log-rank, P=0.011) and age at hospital admission (Log-rank, P=0.046, Table IV and FIG. III). During hospitalization, 76 COVID-19 patients (44.2%) were transferred to the ICU (severe COVID-19) immediately upon admission. Of these, 23 (30.3%) were treated with non-invasive ventilation, 53 (69.7%) with invasive ventilation, and 21 (27.6%) underwent extracorporeal membrane oxygenation. When compared to severity-matched SARS-CoV-2-negative controls, patients with severe COVID-19 had on average longer ICU stays (mean 13 [IQR, 7-35] days versus mean 8 [IQR, 3-15] days, P<0.0001), were more likely to display acute respiratory distress syndrome (ARDS, P=0.0195), and had overall worse oxygenation, requiring higher FiO2 (P<0.0001) as well as having lower PaO₂/FiO₂ ratios (P=0.0003, Table V). Interestingly, although many cardiovascular metrics were unchanged, admitted COVID-19 patients had an increase in the number of noted arrhythmic events (P=0.007) and a higher number of secondary cardiovascular events (P=0.001, Table V, Defined in Supplemental Methods). However, when compared to severity matched SARS-CoV-2 negative patients, there were no differences in overall survival (P=0.42; FIG. 1B).

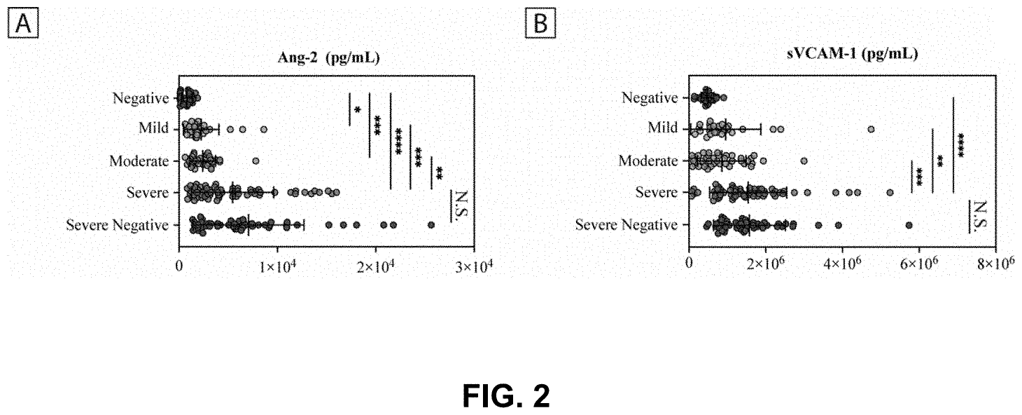

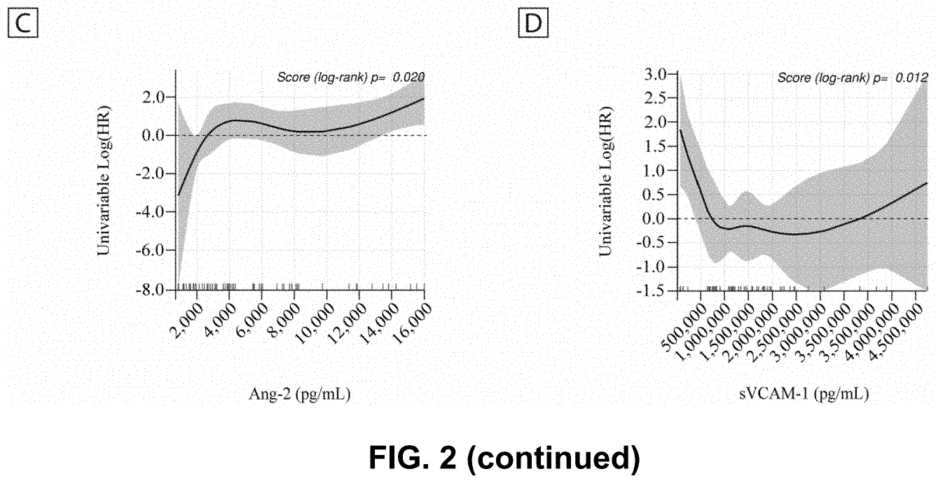

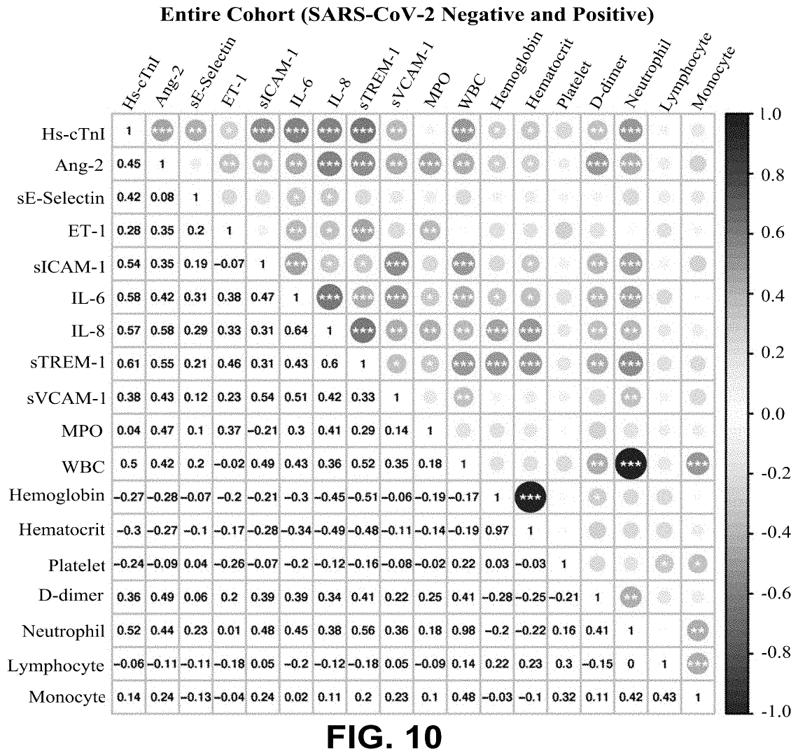

Associations of Cardiovascular and Inflammatory Biomarkers with Outcomes: The vascular endothelium acts as the crucial interface between blood components and tissues, displaying a series of properties that maintain homeostasis (i.e., maintenance of vascular barrier, coagulative capacity, and modulation of immunological responses).(22, 37, 38) While these functions participate in the dynamic regulation of cardiovascular function and coordinate many host defense mechanisms, proinflammatory cytokines may elicit a change in endothelial phenotype, promoting thrombosis, local tissue injury, propagating inflammation and potentially contributing to mortality.(39) In order to test the hypothesis that metrics of cardiac damage and inflammatory endothelial dysfunction/activation may better reflect COVID-19 severity and subsequent mortality than standard clinical metrics, a custom Simple Plex assay for markers robustly associated with inflammation and endothelial dysfunction was performed (i.e., Ang-2, endothelin-1 [ET-1], soluble intercellular adhesion molecule [sICAM], soluble vascular cell adhesion molecule [sVCAM], soluble E-Selectin [sE-selectin], sTREM-1, interleukin-6 [IL], and IL-8), along with standalone assays measuring myeloperoxidase (MPO) and high-sensitivity cardiac troponin (hs-cTnI). Correlation analysis revealed that Ang-2, sE-Selectin, and sICAM had moderate correlations with numerous other inflammatory markers, with correlation coefficients ranging from −0.89 (IL-6:MPO) to 0.89 (sICAM-1:Ang-2; FIGS. 10-15). Myeloperoxidase was the only biomarker without a significant correlation. Among these markers, two were significantly different between mild SARS-CoV-2 positive patients and SARS-CoV-2 negative patients with mild illness at t₀₋₁ (elevated Ang-2, P=0.0200; elevated MPO, P=0.0439, FIG. 2A, B, Figure X). In contrast, nine of the markers (Ang-2, ET-1, sICAM-1, sVCAM-1, sE-Selectin, sTREM-1, IL-6, IL-8, and MPO, FIG. 2A, B, Figure X) reflected differences among severity within the SARS-CoV-2 positive cohort but failed to demonstrate significance between the critically ill patient groups that did or did not have COVID-19.

Amongst all patients admitted with COVID-19, univariable analysis revealed that only Ang-2 was associated with mortality (P=0.015; Table IV), suggesting higher concentrations are associated with higher mortality, while both Ang-2 (P=0.020) and sVCAM-1 (P=0.012) were associated with mortality when looking specifically at the severe COVID-19 patients (FIG. 2C, D, Table VI). Sub-stratifying these markers by severity, there were significantly higher t₀₋₁ concentrations of Ang-2, IL-6, and MPO in non-survivors with COVID-19 when compared to the severe SARS-CoV-2 negative patients (Figure XI). Over time, only IL-6 and MPO remained significantly different between severe COVID-19 and severe negative patients (Figure XII and XIII). Taken together, while markers of inflammation/endothelial dysfunction were observed at early timepoints and associated with severity, the majority were not specific to either COVID-19 status or mortality.

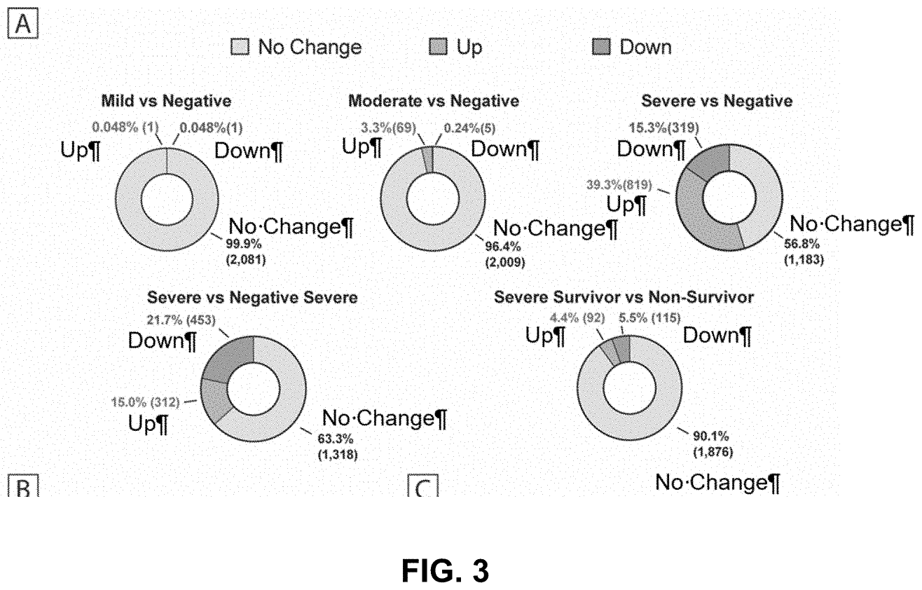

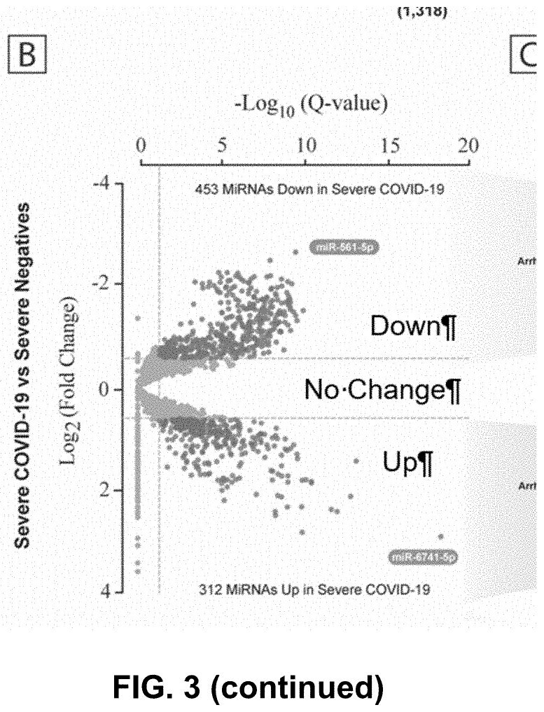

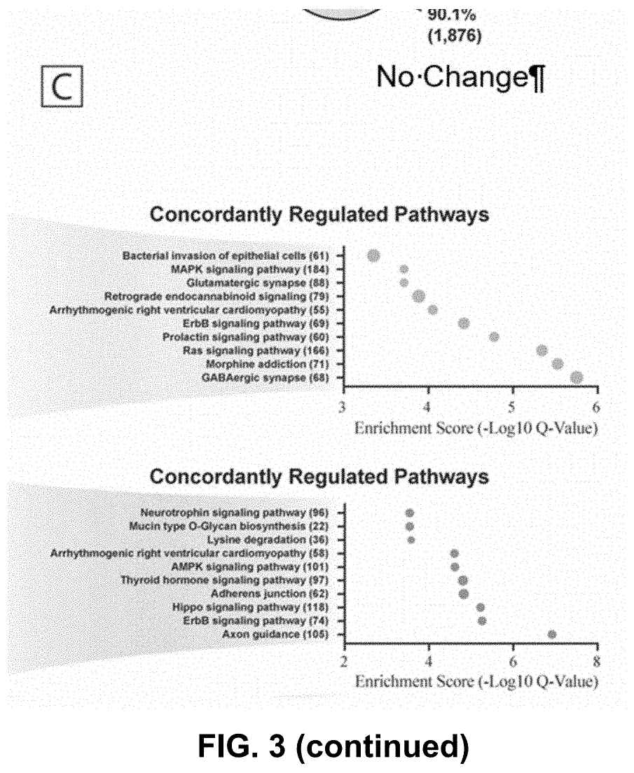

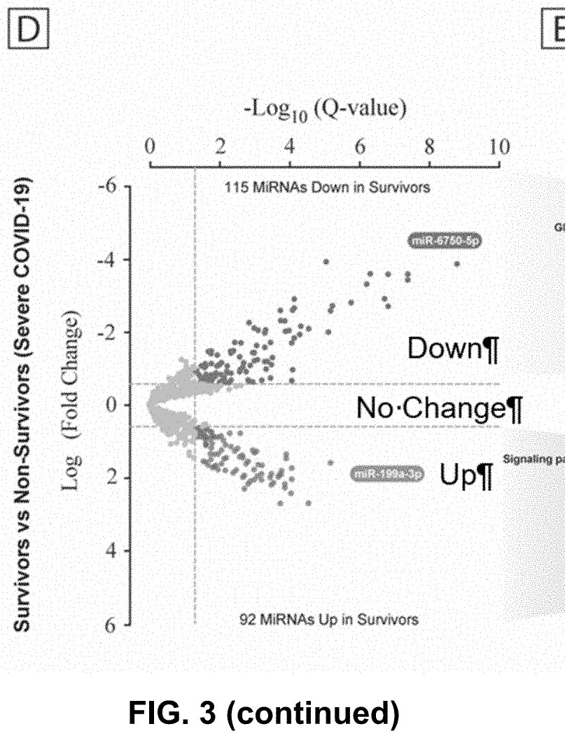

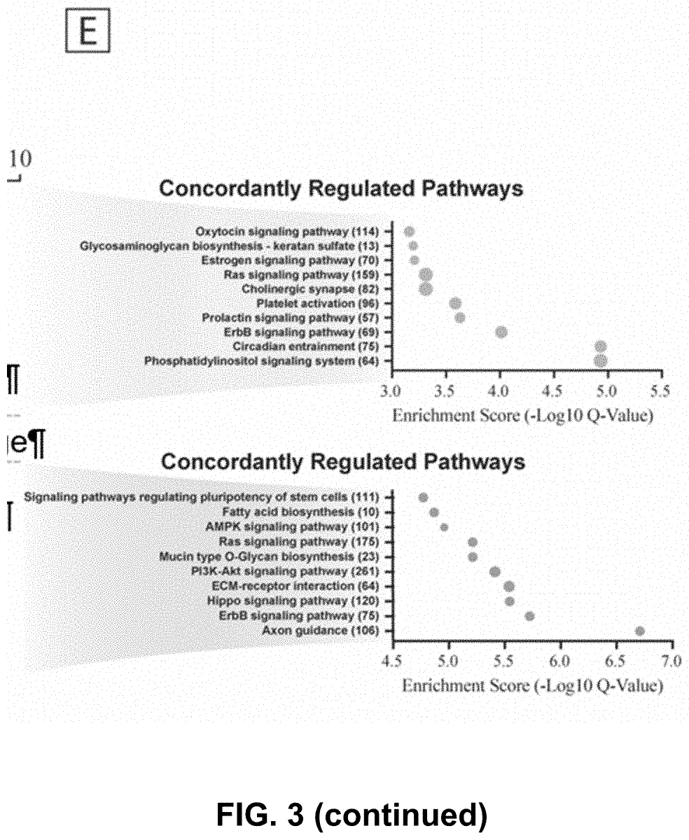

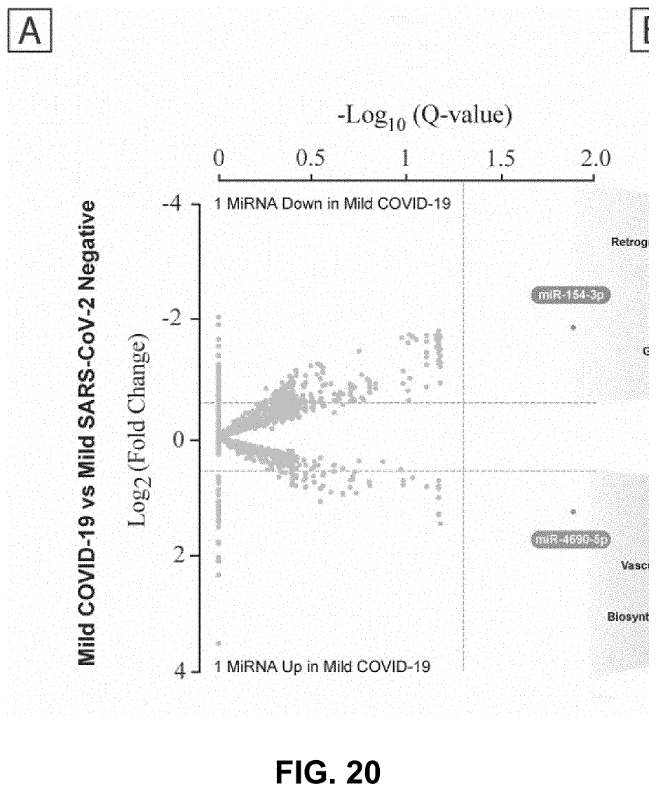

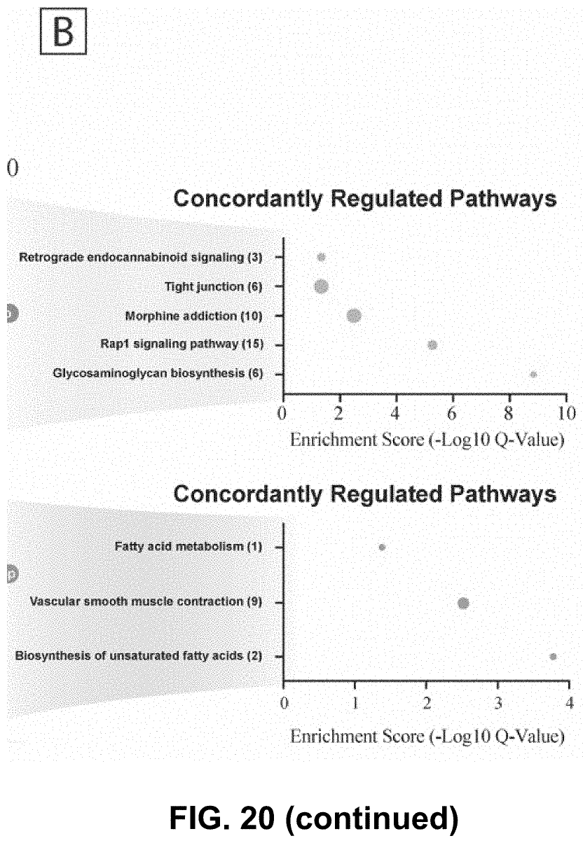

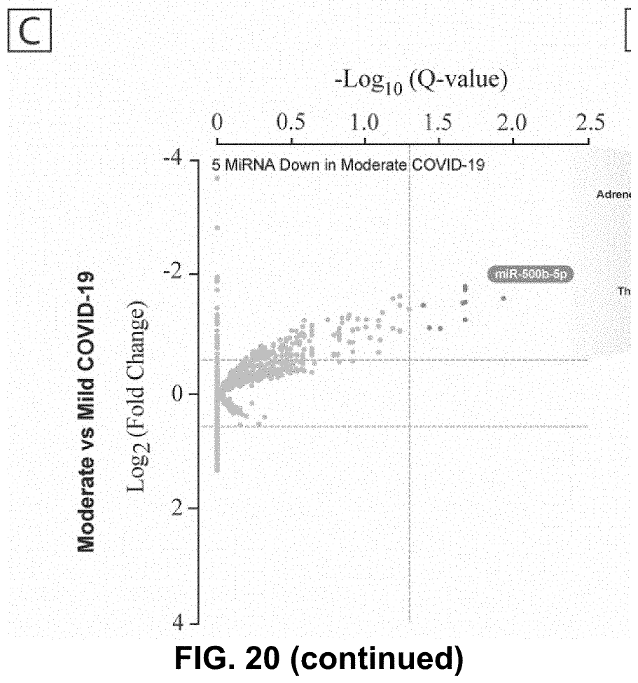

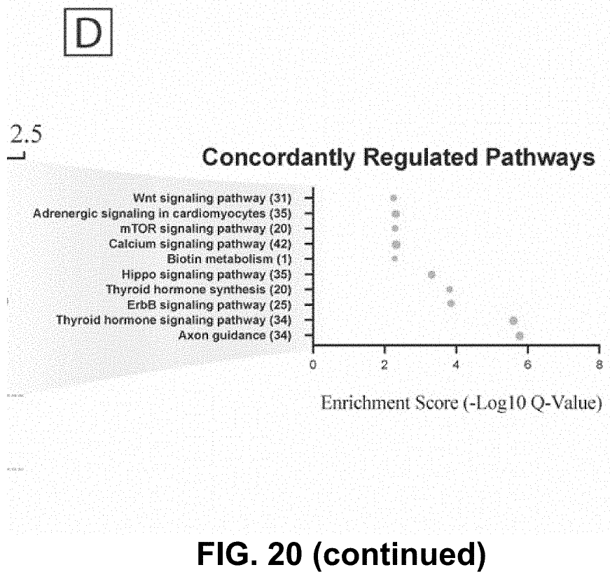

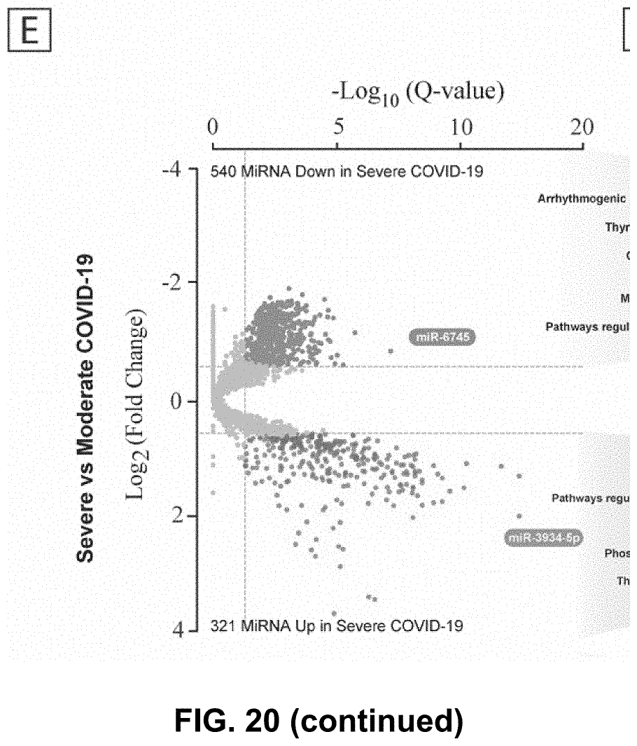

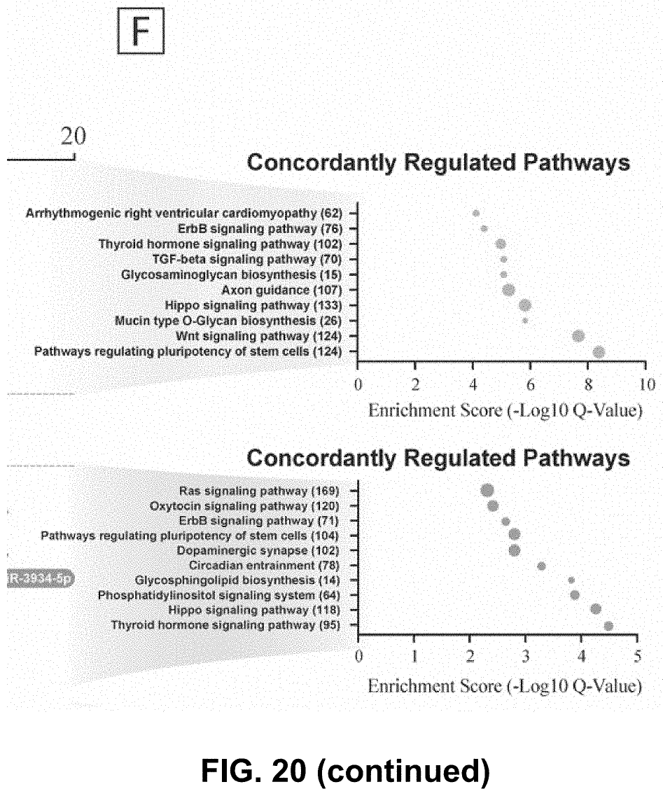

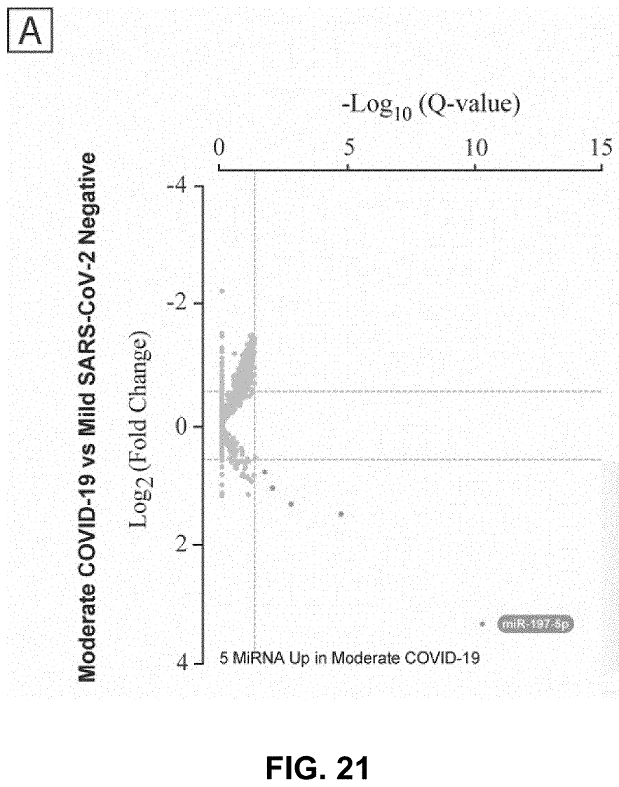

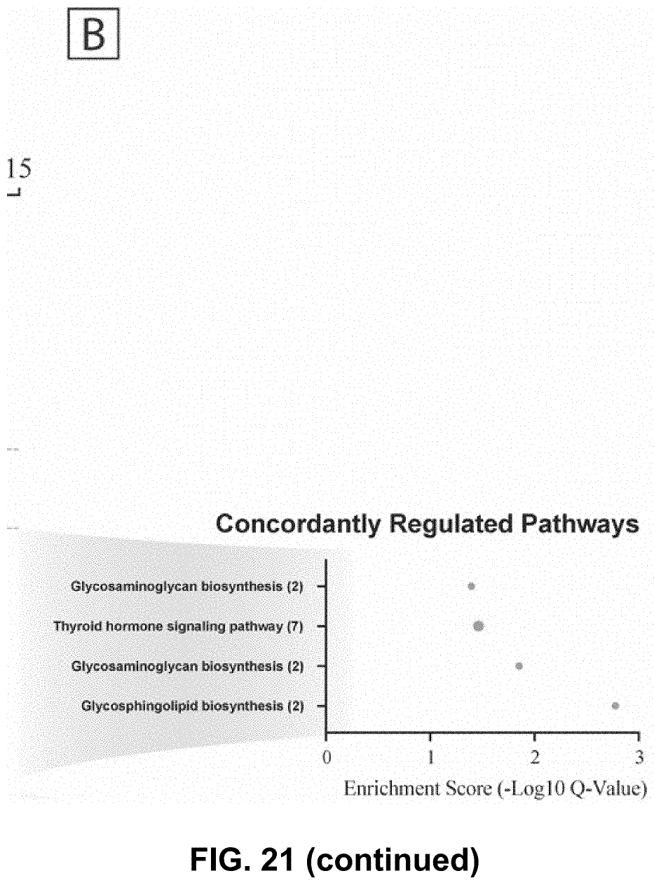

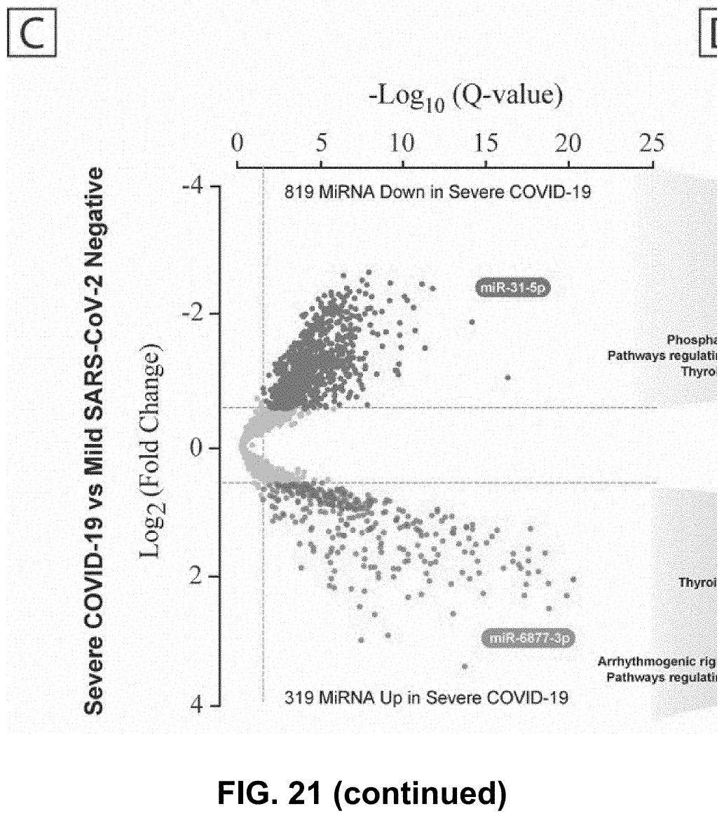

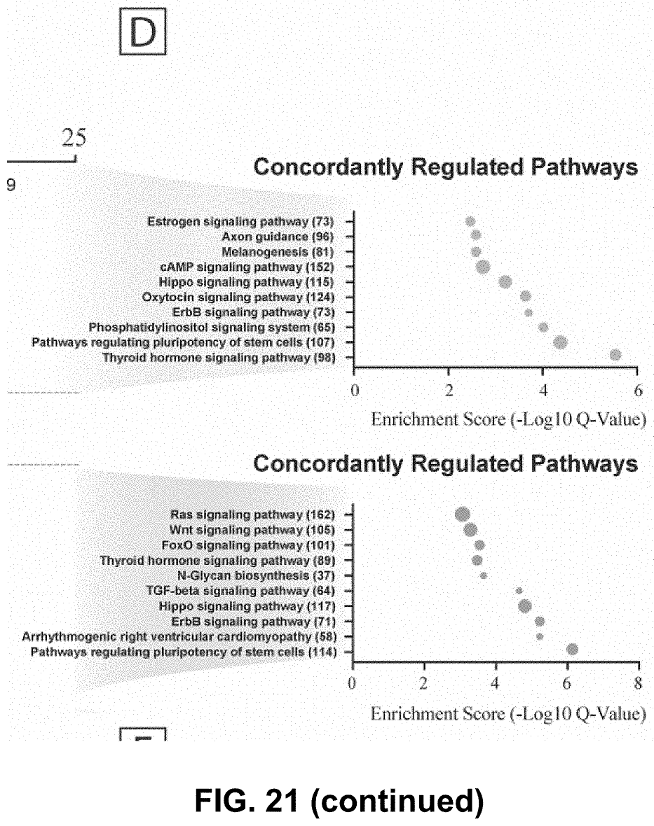

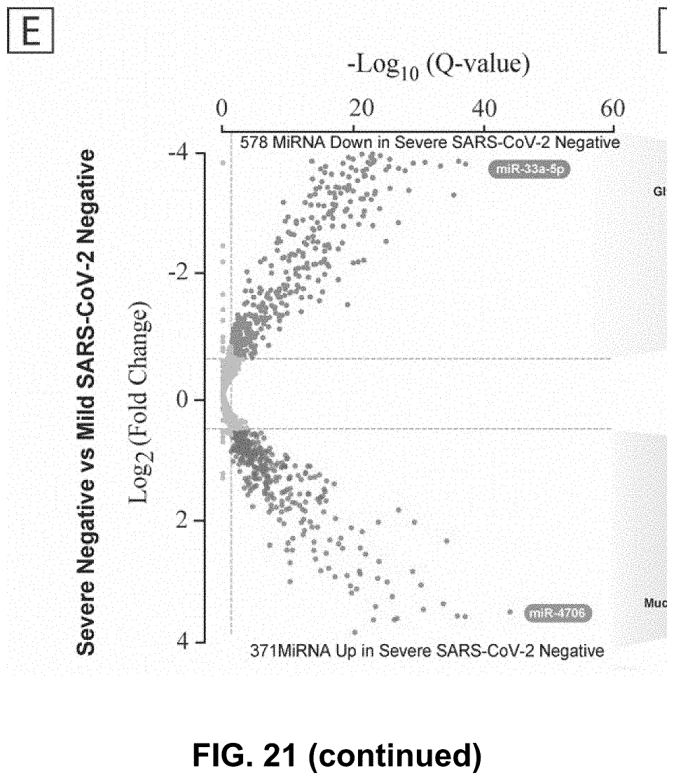

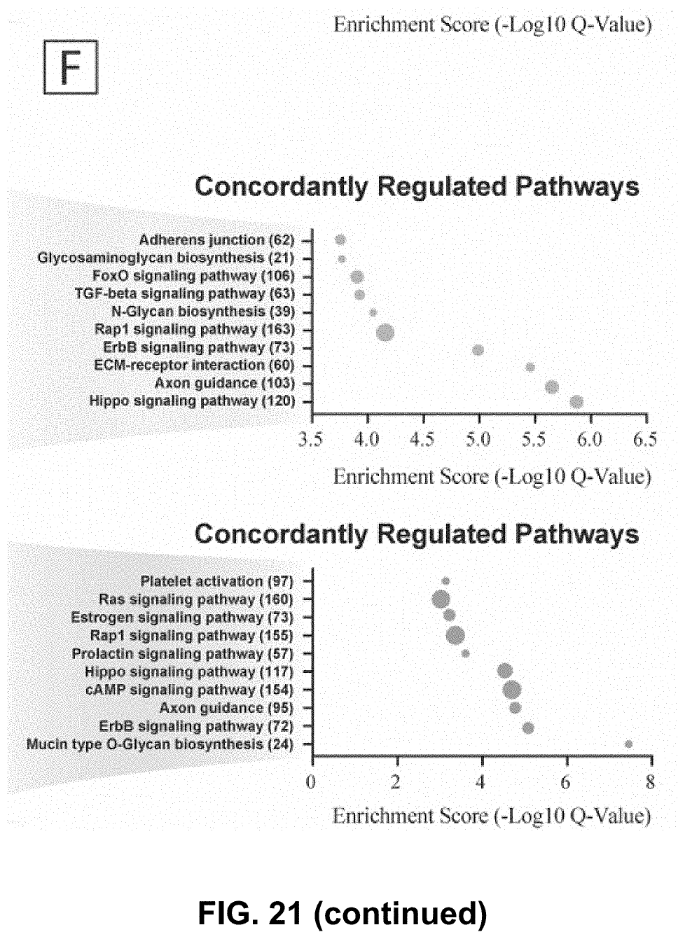

Plasma MiRNA Atlas of SARS-CoV-2 Infection May Reveal Markers Specific to COVID-19 and Mortality: While select inflammatory/EC activation markers were informative for ICU mortality, they may lack the ability to distinguish between COVID-19 and non-COVID-19 pathology. Assessment of the circulating miRNA transcriptome may provide further precision with respect to patient subgroups, since miRNA profiling (in contrast to circulating protein markers) has been shown to effectively differentiate complex disease etiologies.(40, 41) Using whole transcriptome miRNA sequencing (2,083 mature miRNAs) plasma obtained from the differing groups of disease severity was screened to identify meaningful differences in miRNA composition (n=30, negative mild; n=14, mild COVID-19; n=15, moderate COVID-19; n=36, severe COVID-19; n=33, negative severe). Comparative analyses indicated that there were substantially higher numbers of differentially expressed miRNA as disease severity progressed (FIG. 3A; Fold ≥±1.5 and Q<0.05). Comparing severe COVID-19 to severe negative patients revealed 765 differentially expressed miRNAs that could subsequently be used for group differentiation (FIG. 3B). DIANA-mirPath pathway analysis on the differentially expressed miRNAs suggested broad enrichment of pathways including those related to cardiomyocyte function (i.e., ErbB2 signaling and arrhythmogenic right ventricular cardiomyopathy) as well as adherens junctions (FIG. 3C). Sub-analysis of the severe COVID-19 patients for mortality revealed 207 differentially expressed miRNA between survivors and non-survivors (FIG. 3D), including pathway enrichment for platelet activation, extracellular matrix-receptor interactions, Ras, and ErbB2 (FIG. 3E). A full list of differentially expressed miRNA and predicted KEGG pathways is available (raw data not presented herein) as well as FIGS. 20 and 21.

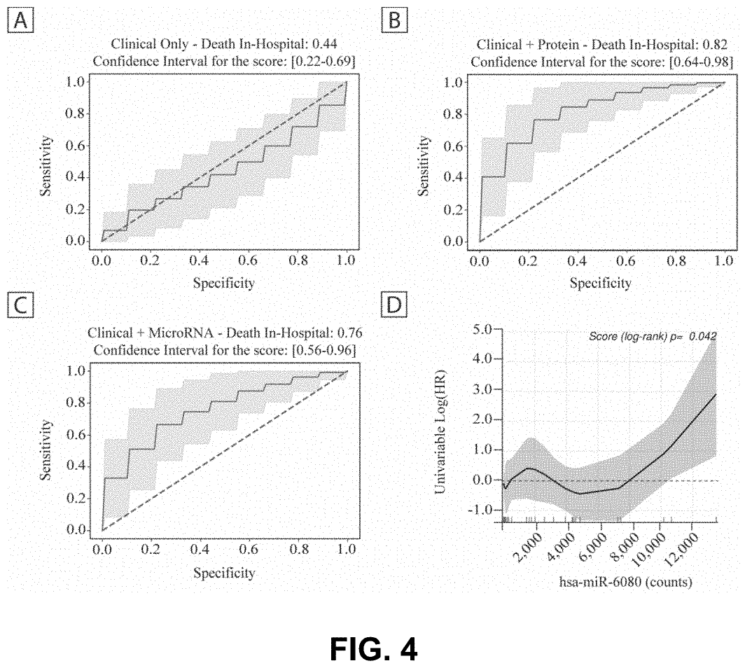

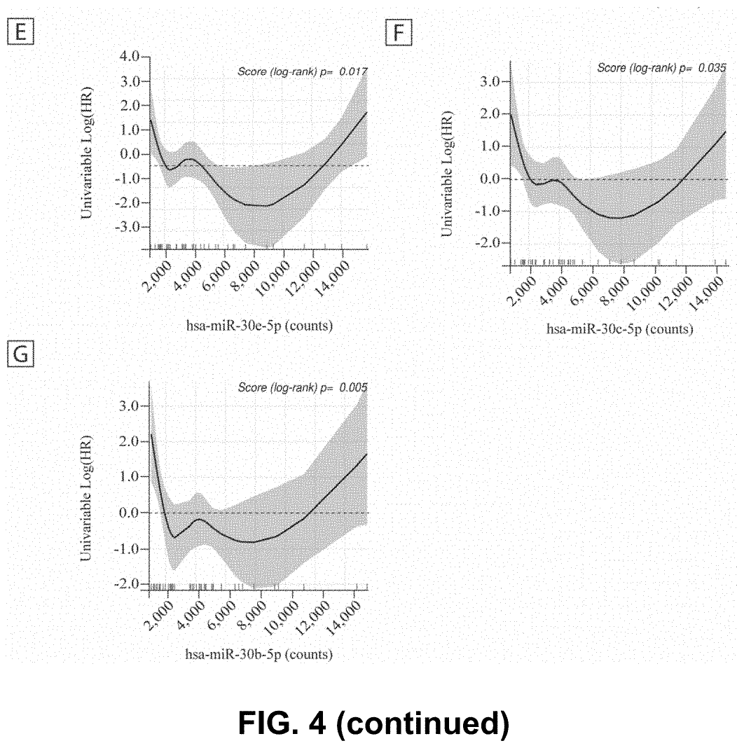

Predictive Power of Clinical, Protein, and miRNA Data on Mortality using Machine Learning: Given the high dimensionality of the dataset generated, the utility of models developed were examined using machine learning to predict in-hospital mortality of COVID-19 patients at admission, based on common clinical data, protein expression data, and miRNA expression data. 250 experiments were performed using repeated randomized stratified sub-sampling cross-validation into disjoint sets of 80% training and 20% testing, to train a set of Random Forest models.(42) Model performance was assessed by the area under the receiver operating characteristic (AUROC) calculated on the testing sets. Aggregate statistics on AUROC were calculated across the 250 experiments. A low AUROC was observed using only clinical features available at the time of admission (AUROC 0.44, 95% CI 0.22-0.69, FIG. 4A).(42) However, incorporation of either the protein expression data or miRNA data improved the performance over the models that used conventional clinical predictors alone, having AUROCs of 0.82 (95% CI 0.64-0.98) and 0.76 (95% CI 0.56-0.96) respectively (FIG. 4B, C). In the clinical data only models, ranking input variables that contributed to model predictivity revealed age and body mass index were amongst the most important (Figure XVI). Furthermore, in contrast to the traditional univariate statistical analyses, MPO, TREM-1, and Ang-2 were listed as the top contributing features in the synthesized clinical and protein multivariate machine learning model, coming before any other traditional clinical factors (Figure XVII). Owing to the exceedingly high dimensionality (2,083 features) machine learning did not highly rank specific variables within the synthesized clinical and miRNA model with statistical reliability.

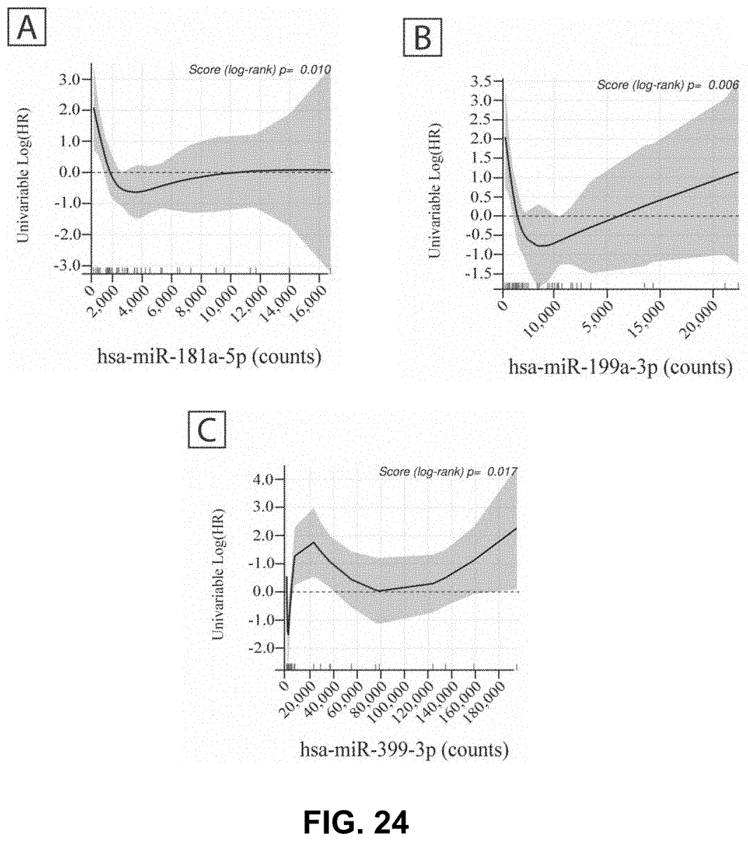

Identification of miRNAs Associated with Severe COVID-19 Mortality: Since miRNAs may have provided superior specificity for COVID-19 mortality compared to protein biomarkers (FIG. 2, 3), candidate miRNA markers that significantly contribute to mortality risk were sought to be identified. miRNAs within the top 50% of abundance were considered and the differential expression between the both survivors and non-survivors of COVID-19 as well as SARS-CoV-2 severe negatives were cross-examined. More so, existing biological relevance was considered, thereby specifically analyzing miR-1 which is associated with myocardial injury(43, 44), miR-199a-3p which has been shown to be cardioprotective(45), miR-181a-5p which has been shown to restrict vascular inflammation(46), along with members of the miR-30 family which are enriched in ECs and capable of modulating inflammation(47-49), as well miR-339-3p and miR-6080 which were among the highest differentially expressed. Univariable hazard ratios and log-rank P-values were generated to determine the relationship of the miRNA expression measured in plasma with mortality. When ranking by significance of independent association with mortality, miRNAs are among the highest ranking factors comparing to other clinical metrics (Table IV and VI) highlighting that miRs-30b/c/e, -6080, -181a-5p, -199a-3p, and -339 (FIG. 4D-E, FIG. 24) are specific for COVID-19 severity and mortality. In contrast, miR-1, failed to show a significant association with mortality (Table VI).

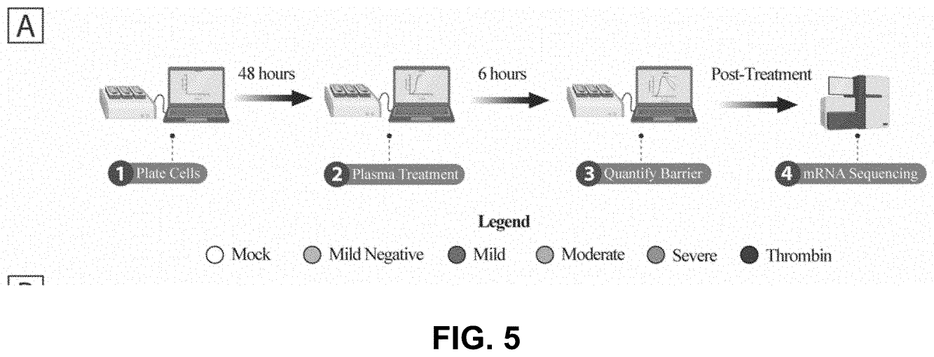

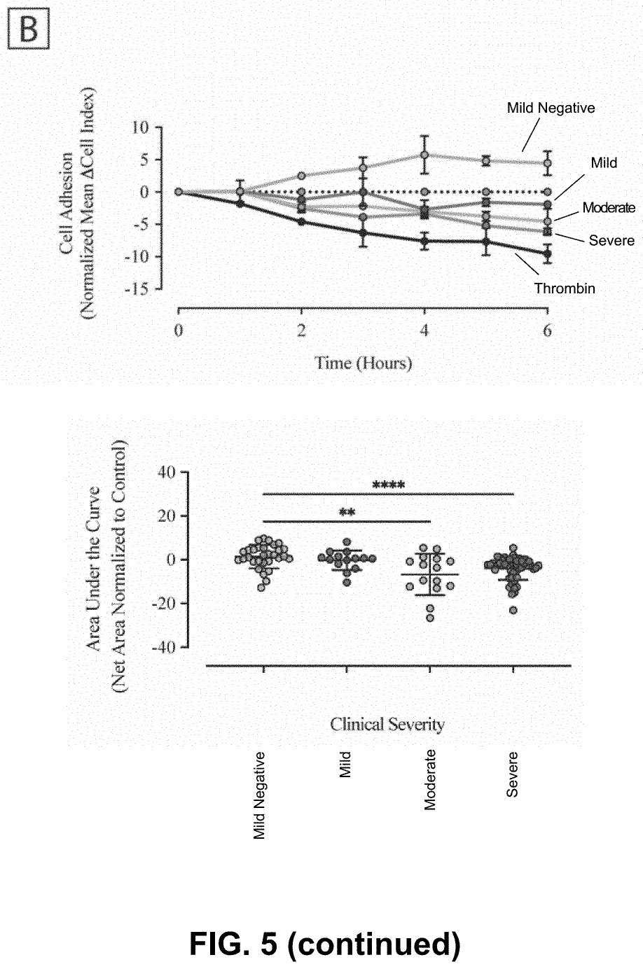

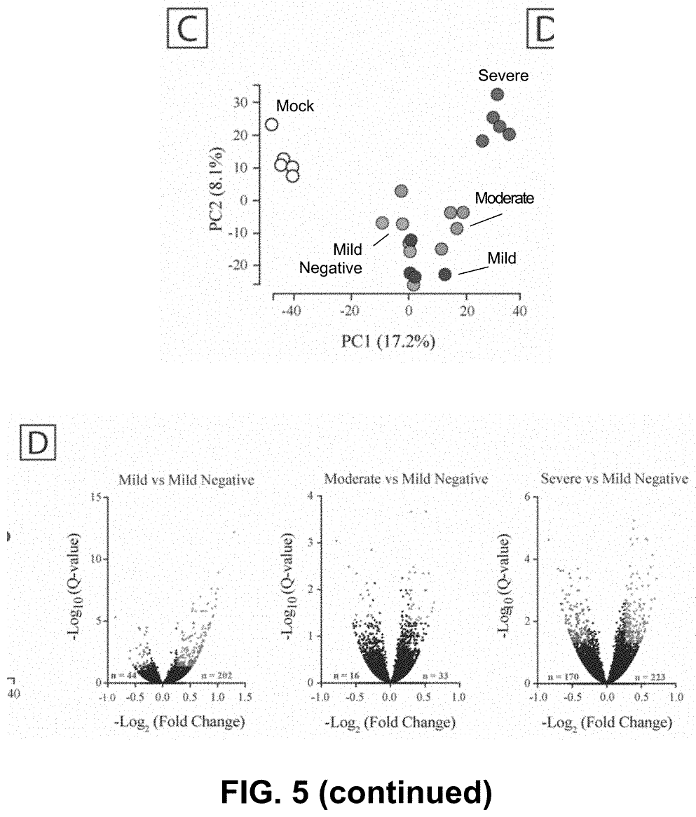

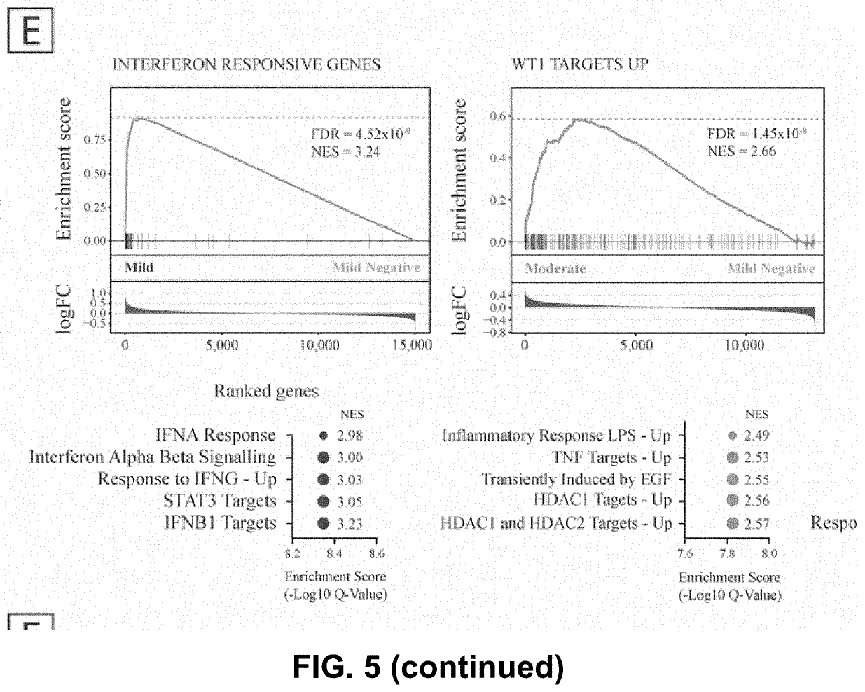

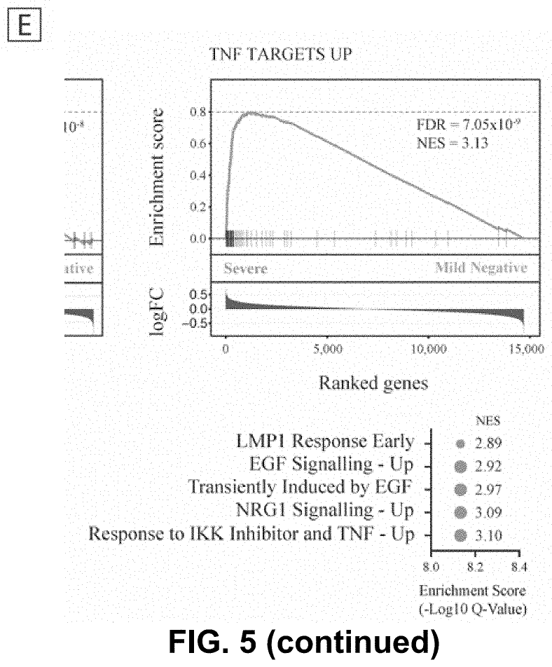

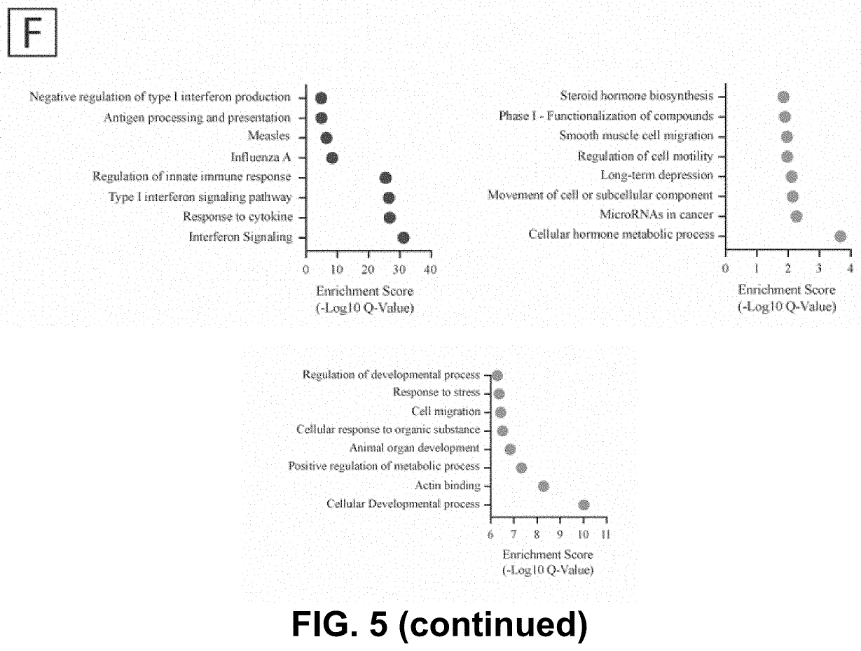

Endothelial Barrier Disruption may be Rapidly Induced During Co-incubation with Plasma from Patients With Moderate to Severe COVID-19: With studies showing conflicting evidence that SARS-CoV-2 can directly infect the endothelium in a physiologically meaningful fashion(12, 50, 51), it was reasoned that the modulation of the extracellular milieu (i.e., changes to circulating plasma components such as sTREM-1, Ang-2, and MPO) as a result of the systemic immune response may be a driving factor behind the observed endothelial dysfunction. In this context, data from previous studies conducted in systemic inflammatory response syndrome and sepsis have underscored the impact of endothelial barrier disruption on disease outcomes.(52) The data presented so far suggested that dysfunction of the endothelium may also accompany COVID-19 and may be associated with poor outcomes. To further examine this hypothesis, two validated permeability platforms using an endothelial monolayer model were used: continuous monitoring of transendothelial electrical resistance (FIG. 5A) using the xCelligence platform, and a transwell system consisting of a confluent monolayer of pooled human umbilical vein ECs on a permeable membrane. In a timeframe that would exclude viral replication(20, 53), cells were treated with 20% (v/v) plasma from the t₀₋₁ samples from across the disease severity spectrum of COVID positive and negative patients. Moderate and severe COVID-19 patient plasma induced significant endothelial barrier dysfunction, while the mild and mild negative patient plasma did not induce significant EC leak in the xCelligence assay (FIG. 5B). Similarly, using a validation cohort, the integrity of the monolayer was gauged through leakage of a large dextran tracer across the EC barrier in both acute (i.e., one-hour) and longer-term (i.e., six-hour) treatment. This may have revealed barrier disruption in response to moderate and severe COVID-19 patient plasma, but not in response to plasma from severe negative patients (Figure XIX). Of the mediators tested, EC permeability correlated with levels of Ang-2, hs-cTnI, ET-1, IL-6, IL-8, sTREM-1, and (Figure XX). These experiments provide in vitro evidence that barrier dysfunction can be independent of direct SARS-CoV-2 infection. It was reasoned that elucidating the unique pathways through which the more severe COVID-19 phenotypes exert their barrier disruptive effects may reveal potential therapeutic approaches to maintain EC barrier.

Modulation of Inflammatory and Cytoskeletal Processes may be Coincident with Barrier Dysfunction: To gain a better understanding of how the endothelium is modulated by plasma components, the gene-level changes using RNA-sequencing of ECs after six hours of co-incubation with patient-derived plasma was investigated. On the transcriptome level (16,285 total quantified genes, having ≥10 reads in at least five samples), biological replicates within groups were tightly correlated (Pearson r=0.975-0.988, n=4-5), suggesting robust intra-group clustering even among a heterogeneous patient group. Principal component analysis of the normalized transcriptome showed segregation between experimental groups, with the severe group being clearly distinct from the mild and moderate groups (FIG. 5C). Pair-wise differential expression analysis with the mild SARS-CoV-2 negative cohort as the control revealed 393, 49, and 246 genes may be differentially expressed (FDR <0.05, log₂ fold change >±0.58) in the mild, moderate, and severe COVID-19 cohorts, respectively (FIG. 5D). Gene set enrichment analysis revealed that co-incubation with either severe or moderate COVID-19 patient plasma altered the expression of endothelial genes related to acute inflammatory response, angiogenic programs, or histone deacetylase activity, whereas administration of plasma from mild COVID-19 patients altered the expression of genes involved in priming of antiviral responses (FIG. 5E and Data Files available and raw data not presented herein). Similarly, pathway enrichment analysis using gProfiler with significant differentially expressed genes highlighted a predominance of KEGG pathways relating to interferon in the mild group, while in contrast, pathways in ECs exposed to moderate and severe COVID-19 plasma related more broadly to cell motility, developmental processes, cell stress responses, cell structure reorganization, and actin mobilization (FIG. 5F). Collectively, these results suggested endothelial structural changes could be occurring that might be amenable to treatment with barrier stabilizing agents.

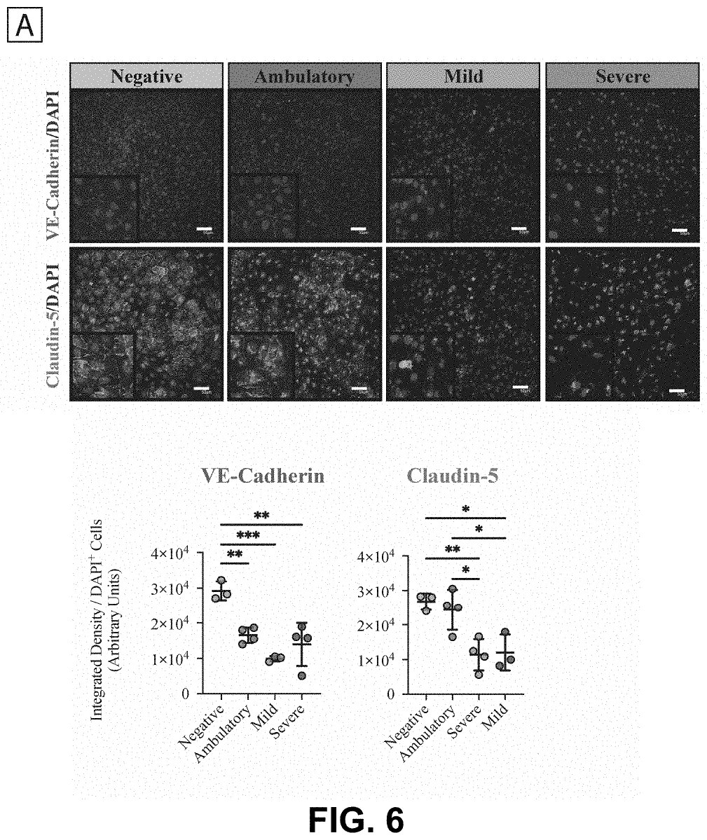

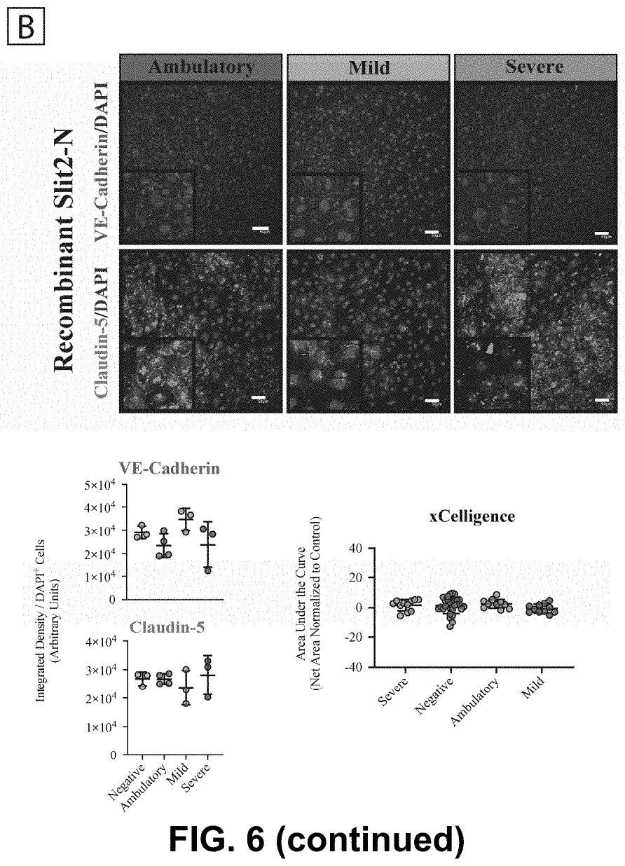

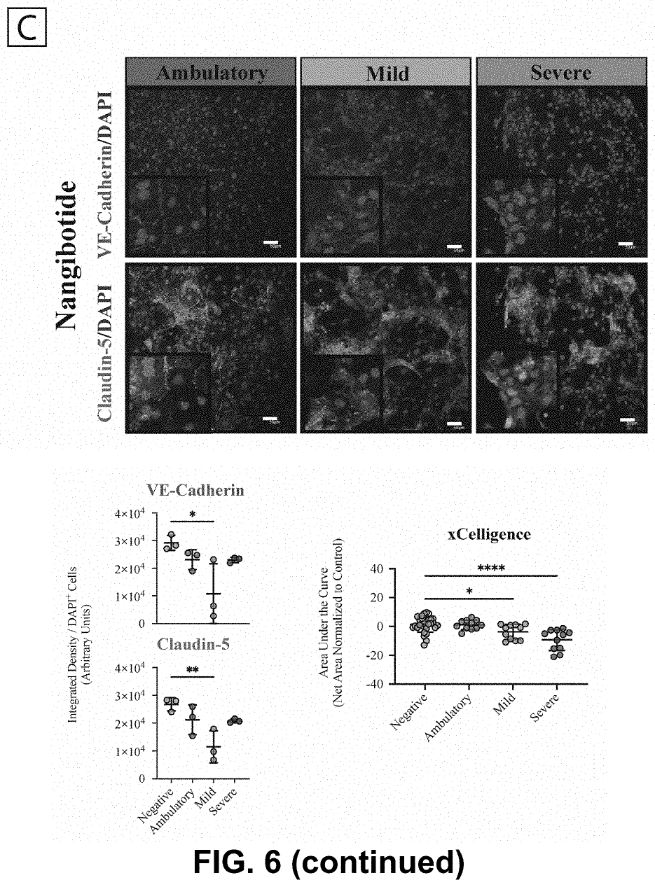

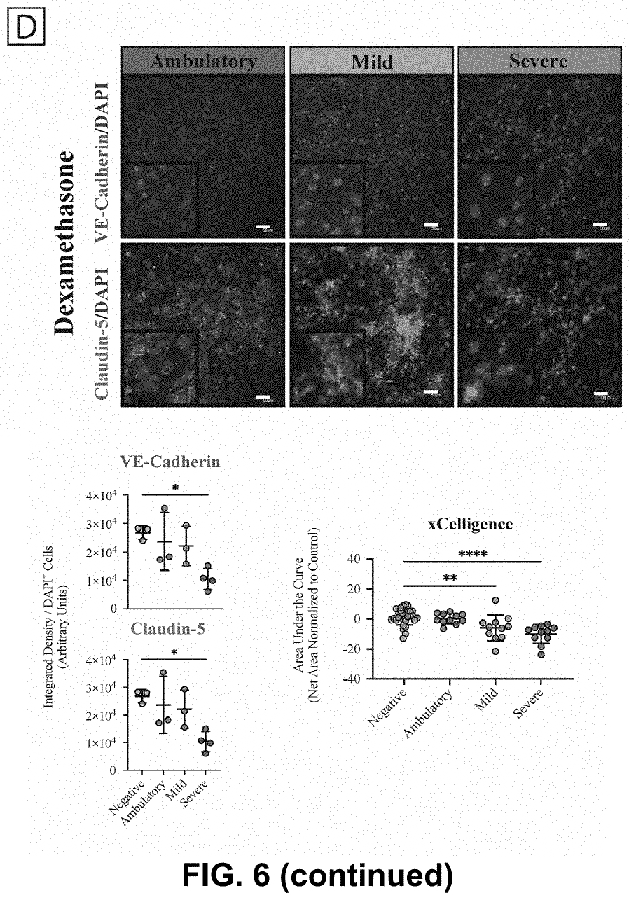

Vascular Barrier Stabilizing Drugs May Prevent EC Permeability Induced by COVID-19 Patient Plasma Exposure in vitro: Two principal structural contributors to the EC barrier were examined, vascular endothelial-cadherin (VE-Cadherin) and Claudin-5. VE-cadherin can be an essential adherens junction protein that regulates cell-cell junctional stability, and Claudin-5 can be a tight junction protein that regulates size-dependent paracellular permeability pathways.(54) Immunostaining confirmed significant disruptions to both VE-cadherin and Claudin-5 expression as well as junctional localization, particularly in ECs exposed to the moderate and severe COVID-19 plasma (FIG. 6A). Whether targeted therapeutics that are known to stabilize the vascular barrier or suppress EC activation can prevent COVID-19 plasma-induced permeability in vitro was examined to gauge the potential clinical importance of these changes in relation to vascular leak. The following drugs that have been reported to reduce endothelial dysfunction were utilized: Slit2-N, a recombinant member of the Slit family of secreted extracellular matrix glycoproteins that stabilizes adherens junctions(55); Nangibotide, a TREM-1 inhibitor(56); and dexamethasone, a potent synthetic adrenal corticosteroid currently used in COVID-19 treatment(57). Co-treatment with Slit2-N prevented the disruption of the EC barrier, as examined through junctional protein expression (FIG. 6B, C, left) and permeability as examined by xCelligence readout (FIG. 6B, C, right). While other agents could maintain junctional protein expression (e.g., Nangibotide, dexamethasone) in specific settings, no other agents were universally able to maintain barrier protein expression in the face of moderate or severe disease plasma exposure and prevent the physiologic barrier disruption measured by electrical resistance.

### Discussion

The induction of substantial acute respiratory distress phenotypes may be driven by a mismatched inflammatory response together with broad vascular dysfunction.(58, 59) While several detailed reports implementing multi-omic approaches have provided insight into the immune cell phenotypes involved in these processes(60-62), risk stratifying immune markers specific to COVID-19 have not been fully elucidated. A comprehensive, multi-omics-based description of the molecular antecedents to COVID-19 mortality, yielding new insights pertaining to the vasculature is provided. The above study further delineates the gradient of vascular dysfunction observed in patients across the spectrum of COVID-19 severity, particularly among those with severe illness. While the findings are consistent with smaller cohort studies examining single markers of cardiovascular dysfunction(26, 63), the results described herein is the first to use proper, disease-negative controls, which asked etiological questions. The analysis may have revealed that although markers of cardiovascular dysfunction (such as Ang-2 and sVCAM-1, FIG. 2A, B) tracked well with COVID-19 severity and outcomes, these markers were similarly elevated in ICU patients without COVID-19, which indicates they may be more appropriate as general markers of disease severity, and they are not COVID-19 specific.

Circulating miRNAs have emerged as exquisite biomarkers for complex pathological conditions, including influenza and sepsis.(64, 65) The inherent stability of plasma microRNAs under harsh conditions and reproducible quantification makes them attractive candidates for use as noninvasive biomarkers.(30) Incorporating whole transcriptome sequencing as an added metric may allow for the effective differentiation between severities of COVID-19 and severe SARS-CoV-2 negative controls using samples gathered on admission. Through this study, further empirical evidence for the value of data from novel biomarkers over metrics traditionally utilized in healthcare systems (i.e., clinical demographics and laboratory data) may have been provided. Notably, the data from electronic medical records fails to adequately capture the risk of in-hospital mortality. Using a Random Decision Forest machine learning model on the multi-omic biologically relevant datasets an exploratory prognostic risk prediction model that incorporates markers that are COVID-19 and vascular specific was developed. Importantly, there are several platforms that allow the interrogation of candidate miRNAs within clinically relevant timeframes.(66, 67) While still investigational, given the extent of pre-clinical and clinical research on miRNAs(40), reasonable to expect miRNA datasets will soon be amenable for clinical implementation. As an example of the importance of a multi-omic approach that includes clinically relevant disease negative controls, miR-1 (which has been intensively studied as a cardiac enriched miRNA and associated with numerous cardiac etiologies(41, 43, 44, 68)) failed to show an association with in-hospital mortality in the study. If the promising miRs-30b/c/e, -6080, -181a-5p, -199a-3p, and -339 identified here are validated in larger populations they would represent new biomarkers that could be utilized for rapid in-hospital risk assessment. As miRNAs represent functional biomarkers, having active roles in gene regulation, they also present an important opportunity to understand the pathophysiological relevance of endothelial-based processes affected by SARS-CoV-2 infection.

Given that the endothelium is the gatekeeper of vascular permeability, which is a main pathophysiological process in systemic inflammatory conditions, there is intense research on their role in ARDS, systemic inflammatory response syndrome, and sepsis (among others).(69-71) Several lines of evidence support the hypothesis that endothelial function is a major determinant of COVID-19 outcome.(63, 72) Endothelial derangements (i.e., pathological sprouting angiogenesis), increased endothelial apoptosis, modified metabolism, and a strong correlation between underlying CVD and mortality may show COVID-19 severity increases.(23, 25, 69) Often forgotten, endothelial dysfunction—associated molecular patterns are broad and strong activators of innate immune responses, leading to innate immunity-mediated organ injury. To this end, plasma from patients with moderate and severe COVID-19 induced profound barrier disruption, as assessed through the modulation of VE-Cadherin and Claudin-5 at EC-EC junctions was observed. Transcriptomic analysis revealed that COVID-19 plasma from moderate to severe patients appears to preferentially induce pro-inflammatory immune gene processes, while plasma from mild patients induces an interferon (IFN) response. This is in line with several studies on the immune response of mild vs severe COVID-19 patients, and confirmed by the over-representation of type I IFN genes in ECs exposed to mild COVID-19 plasma.(63, 73, 74) Type I IFNs are critical mediators of the antiviral immune response and subverting the early type I IFN response has been shown repeatedly to be a contributor to coronavirus infection.(73) Looking at the moderate and severe phenotypes where endothelial pro-inflammatory responses (e.g., TNF) and histone deacetylase dysregulation occurred, both epigenetic modifications as well as TNF responses have been documented in COVID-19.(63, 74)

Increased pro-inflammatory cytokine expression, specifically TNF and IL-6, has been shown to upregulate trypsin, resulting in the loss of endothelial tight junctions, subsequently inducing vascular hyperpermeability.(75, 76) Based on the above study, recombinant Slit2-N, may effectively prevent the induction of endothelial permeability in vitro. Additionally, measurement of the levels of endogenous sSlit2 in SARS-CoV-2 patient plasma revealed a significant upregulation only in patients with severe disease suggesting a possible compensatory mechanism and furthering the biological relevance of Slit2-N treatment (Figure XXI). Therefore, as a proof-of-concept, these data support the hypothesis that disruption of endothelial barrier function, particularly as a result of loss of adherens and tight junctions, is induced during COVID-19. These findings support a model in which systemic induction of pro-inflammatory processes, rather than direct infection, may be the primary driver of systemic endothelial perturbations observed in COVID-19 patients, and that relative to current treatments aimed at dampening immune responses, preserving endothelial barrier may be a viable adjuvant to reduce the mortality of SARS-CoV-2.

It will nonetheless be important to determine whether pre-existing and potentially chronic endothelial permeability—layered on top of a permeabilizing stimuli such as infection—can be reversed through biologics that interact directly at the endothelial interface. If the significant induction of endothelial permeability can be overridden, even in patients with an ongoing inflammatory insult, then pharmacological intervention at the level of the vasculature may be an effective adjuvant therapy for patients at a higher risk of adverse events. Pharmacological treatments that enhance vascular resiliency in the face of an aberrant host immune response may be a therapeutically feasible approach for managing vascular dysfunction without compromising immunity. Although stabilizing the vasculature may be more practical than attenuating individual cytokines, it remains to be determined whether severe vascular damage can be reversed in a clinically meaningful way. Adrenomedullin, a peptide hormone capable of endothelial stabilization, may have high in vivo efficacy and positive results from both Phase I and II trials in patients with sepsis.(80, 81) A prospective study comparing endothelial function in critical illness of non-viral etiology and viral etiology may provide clearer insights as to the translatability of these findings.

Taken together, herein is provided the first highly salient comparison of a diverse group of cardiovascular markers between COVID-19 positive and negative patients, highlighting that well-known markers of inflammation, cardiac damage, and endothelial dysfunction are not specific to COVID-19 pathology. Notably, with a targeted miRNA transcriptomic approach, specific markers that may show better discrimination between these two groups were discerned. Using machine learning, incorporation of protein and miRNA markers improved the prediction of in-hospital mortality over baseline clinical variables. While exploratory, this is a clinically feasible approach that has the advantage of using pathophysiologically relevant SARS-CoV-2's markers, as opposed to the surrogate markers used in most other published risk stratification models.(10, 26) Finally, the data provide several lines of evidence supporting the notion that endothelial barrier function is affected in a SARS-CoV-2 specific manner, that is distinct from the pathways involved in critically ill patients with non-COVID-19 severe respiratory illnesses. The data reinforce the idea that barrier dysfunction is likely independent of direct viral infection and instead secondary to yet undiscovered mediators in the plasma. These observations were further pursued using assays of EC function using an in vitro model, which served as a platform for rational therapeutic choice. Here, EC barrier may be reduced by the addition of COVID-19 patient plasma in a disease severity-dependent manner and that this can be prevented by stabilization with Slit2-N was shown. Together, the work provides biological insight into the role of the endothelium in SARS-CoV-2 infection, the importance of miRNA as disease- and pathway-specific biomarkers, and the exciting possibility that endothelial barrier stabilizing treatments might hold promise in COVID-19. Moreover, the work provides insights into the use of this approach to find therapeutic options that might prove useful in other critical illnesses and emerging infectious diseases where endothelial permeability is central to disease pathophysiology.

Limitations

The results of this study should be viewed in the context of its design, sample size, and socio-geographical context (i.e., access to advanced care life support). Variation between studies may represent temporal differences in circulating viral strains or vaccination status (notably, this study was done in a timeframe exclusive of B.1.1.7 [Alpha], B.1.351 [Beta], B.1.617.2 [Delta], and P.1 [Gamma] circulating strains and prior to public vaccination programs), which can impact severity and mortality. Findings from the miRNA atlas and machine learning algorithms should be validated in additional cohorts before implementation in clinical practice. The in vitro system used in this manuscript represents a reductionist approach to disease modelling, and as such, vascular cell co-culture or examination of specific endothelial beds (i.e., coronary or pulmonary) may better inform microenvironment changes in COVID-19. Putative therapies could be further assessed in co-culture models (e.g., organ on a chip) that better mimic tissue complexity and will require testing in animal models. Nevertheless, the findings provide important knowledge relating to the pathophysiology and risk stratification of betacoronavirus infection and provide avenues for future research in infectious disease.

Perspectives (Clinical Competencies/Translational Outlook)

Bedside microRNA-based diagnostics could significantly enhance clinical care of those with acute care diseases.

Vascular stabilizing therapies represent an attractive supplemental modality for treating critical illnesses and emerging infectious diseases where endothelial permeability is central to disease pathophysiology and should be further explored.

### REFERENCES

1. WHO. Novel Coronavirus—China. 2020.
2. Zhou F, Yu T, Du R, Fan G, Liu Y, Liu Z, et al. Clinical course and risk factors for mortality of adult inpatients with COVID-19 in Wuhan, China: a retrospective cohort study. Lancet. 2020; 395(10229):1054-62.
3. Wu Z, and McGoogan J M. Characteristics of and Important Lessons From the Coronavirus Disease 2019 (COVID-19) Outbreak in China: Summary of a Report of 72 314 Cases From the Chinese Center for Disease Control and Prevention. JAMA. 2020; 323(13):1239-42.
4. Challen R, Brooks-Pollock E, Read J M, Dyson L, Tsaneva-Atanasova K, and Danon L. Risk of mortality in patients infected with SARS-CoV-2 variant of concern 202012/1: matched cohort study. bmj. 2021; 372.
5. Liu F-Y, Sun X-L, Zhang Y, Ge L, Wang J, Liang X, et al. Evaluation of the risk prediction tools for patients with coronavirus disease 2019 in Wuhan, China: A single-centered, retrospective, observational study. Critical care medicine. 2020.
6. Kar S, Chawla R, Haranath S P, Ramasubban S, Ramakrishnan N, Vaishya R, et al. Multivariable mortality risk prediction using machine learning for COVID-19 patients at admission (AICOVID). Scientific reports. 2021; 11(1):1-11.
7. Wu Z, and McGoogan J M. Characteristics of and Important Lessons From the Coronavirus Disease 2019 (COVID-19) Outbreak in China: Summary of a Report of 72 314 Cases From the Chinese Center for Disease Control and Prevention. Jama. 2020.
8. Rhee C, Jones T M, Hamad Y, Pande A, Varon J, O'Brien C, et al. Prevalence, Underlying Causes, and Preventability of Sepsis-Associated Mortality in US Acute Care Hospitals. JAMA Network Open. 2019; 2 (2):e187571-e.
9. Shi S, Qin M, Shen B, Cai Y, Liu T, Yang F, et al. Association of Cardiac Injury With Mortality in Hospitalized Patients With COVID-19 in Wuhan, China. JAMA Cardiol. 2020; 5(7):802-10.
10. Cosyns B, Lochy S, Luchian M L, Gimelli A, Pontone G, Allard S D, et al. The role of cardiovascular imaging for myocardial injury in hospitalized COVID-19 patients. European Heart Journal-Cardiovascular Imaging. 2020; 21(7):709-14.
11. Lamb Y N. Remdesivir: first approval. Drugs. 2020:1-9.
12. Varga Z, Flammer A J, Steiger P, Haberecker M, Andermatt R, Zinkernagel A S, et al. Endothelial cell infection and endotheliitis in COVID-19. The Lancet. 2020; 395(10234):1417-8.
13. Fox S E, Akmatbekov A, Harbert J L, Li G, Brown J Q, and Vander Heide R S. Pulmonary and cardiac pathology in African American patients with COVID-19: an autopsy series from New Orleans. The Lancet Respiratory Medicine. 2020; 8(7):681-6.
14. Zhang H, Penninger J M, Li Y, Zhong N, and Slutsky A S. Angiotensin-converting enzyme 2 (ACE2) as a SARS-CoV-2 receptor: molecular mechanisms and potential therapeutic target. Intensive Care Med. 2020; 46(4):586-90.
15. Wang K, Gheblawi M, and Oudit G Y. Angiotensin Converting Enzyme 2. Circulation. 2020; 142(5):426-8.
16. Patel V B, Zhong J-C, Grant M B, and Oudit G Y. Role of the ACE2/angiotensin 1-7 axis of the renin—angiotensin system in heart failure. Circulation research. 2016; 118(8):1313-26.
17. Zhong J, Basu R, Guo D, Chow F L, Byrns S, Schuster M, et al. Angiotensin-converting enzyme 2 suppresses pathological hypertrophy, myocardial fibrosis, and cardiac dysfunction. Circulation. 2010; 122(7):717-28, 18 p following 28.
18. Zhao Y, Zhao Z, Wang Y, Zhou Y, Ma Y, and Zuo W. Single-Cell RNA Expression Profiling of ACE2, the Receptor of SARS-CoV-2. Am J Respir Crit Care Med. 2020; 202(5):756-9.
19. Clausen T M, Sandoval D R, Spliid C B, Pihl J, Painter C D, Thacker B E, et al. SARS-CoV-2 Infection Depends on Cellular Heparan Sulfate and ACE2. bioRxiv. 2020:2020.07.14.201616.
20. McCracken I R, Saginc G, He L, Huseynov A, Daniels A, Fletcher S, et al. Lack of Evidence of ACE2 Expression and Replicative Infection by SARSCoV-2 in Human Endothelial Cells. Circulation. 2021.
21. Clerkin K J, Fried J A, Raikhelkar J, Sayer G, Griffin J M, Masoumi A, et al. COVID-19 and Cardiovascular Disease. Circulation. 2020; 141(20):1648-55.
22. Gustafson D, Raju S, Wu R, Ching C, Veitch S, Rathnakumar K, et al. Overcoming Barriers. Arteriosclerosis, Thrombosis, and Vascular Biology. 2020; 40(8):1818-29.
23. Goshua G, Pine A B, Meizlish M L, Chang C H, Zhang H, Bahel P, et al. Endotheliopathy in COVID-19-associated coagulopathy: evidence from a single-centre, cross-sectional study. Lancet Haematol. 2020; 7 (8):e575-e82.
24. Middleton E A, He X-Y, Denorme F, Campbell R A, Ng D, Salvatore S P, et al. Neutrophil extracellular traps contribute to immunothrombosis in COVID-19 acute respiratory distress syndrome. Blood. 2020; 136(10):1169-79.
25. Smadja D M, Guerin C L, Chocron R, Yatim N, Boussier J, Gendron N, et al. Angiopoietin-2 as a marker of endothelial activation is a good predictor factor for intensive care unit admission of COVID-19 patients. Angiogenesis. 2020; 23(4):611-20.
26. Van Singer M, Brahier T, Ngai M, Wright J, Weckman A M, Erice C, et al. COVID-19 risk stratification algorithms based on sTREM-1 and IL-6 in emergency department. J Allergy Clin Immunol. 2020.
27. Langley R J, Tsalik E L, Velkinburgh J Cv, Glickman S W, Rice B J, Wang C, et al. An Integrated Clinico-Metabolomic Model Improves Prediction of Death in Sepsis. Science Translational Medicine. 2013; 5(195):195ra95-ra95.
28. Govaere O, Cockell S, Tiniakos D, Queen R, Younes R, Vacca M, et al. Transcriptomic profiling across the nonalcoholic fatty liver disease spectrum reveals gene signatures for steatohepatitis and fibrosis. Science Translational Medicine. 2020; 12 (572):eaba4448.
29. Vegter E L, Schmitter D, Hagemeijer Y, Ovchinnikova E S, van der Harst P, Teerlink J R, et al. Use of biomarkers to establish potential role and function of circulating microRNAs in acute heart failure. International journal of cardiology. 2016; 224:231-9.
30. Gustafson D, Tyryshkin K, and Renwick N. microRNA-guided diagnostics in clinical samples. Best Pract Res Clin Endocrinol Metab. 2016; 30(5):563-75.
31. Health. Nlo. COVID-19 Treatment Guidelines Panel. Coronavirus Disease 2019 (COVID-19) Treatment Guidelines. 2021.
32. BioCarta. Biotech Software & Internet Report. 2001; 2(3):117-20.
33. Kanehisa M, and Goto S. KEGG: kyoto encyclopedia of genes and genomes. Nucleic Acids Res. 2000; 28(1):27-30.
34. Jassal B, Matthews L, Viteri G, Gong C, Lorente P, Fabregat A, et al. The reactome pathway knowledgebase. Nucleic Acids Res. 2020; 48 (D1):D498-d503.
35. Team R C. R: A language and environment for statistical computing. 2020.
36. Schindelin J, Arganda-Carreras I, Frise E, Kaynig V, Longair M, Pietzsch T, et al. Fiji: an open-source platform for biological-image analysis. Nature Methods. 2012; 9(7):676-82.
37. Libby P, and Lüscher T. COVID-19 is, in the end, an endothelial disease. European Heart Journal. 2020; 41(32):3038-44.
38. Teuwen L A, Geldhof V, Pasut A, and Carmeliet P. COVID-19: the vasculature unleashed. Nat Rev Immunol. 2020; 20(7):389-91.
39. Katz S D, Hryniewicz K, Hriljac I, Balidemaj K, Dimayuga C, Hudaihed A, et al. Vascular endothelial dysfunction and mortality risk in patients with chronic heart failure. Circulation. 2005; 111(3):310-4.
40. Blanco-Dominguez R, Sanchez-Diaz R, de la Fuente H, Jimenez-Borreguero L J, Matesanz-Marín A, Relaño M, et al. A Novel Circulating MicroRNA for the Detection of Acute Myocarditis. New England Journal of Medicine. 2021; 384(21):2014-27.
41. Ovchinnikova E S, Schmitter D, Vegter E L, Ter Maaten J M, Valente M A, Liu L C, et al. Signature of circulating microRNAs in patients with acute heart failure. Eur J Heart Fail. 2016; 18(4):414-23.
42. Breiman L. Random forests. Machine learning. 2001; 45(1):5-32.
43. Yang B, Lin H, Xiao J, Lu Y, Luo X, Li B, et al. The muscle-specific microRNA miR-1 regulates cardiac arrhythmogenic potential by targeting GJA1 and KCNJ2. Nature medicine. 2007; 13(4):486-91.
44. Liu X, Fan Z, Zhao T, Cao W, Zhang L, Li H, et al. Plasma miR-1, miR-208, miR-499 as potential predictive biomarkers for acute myocardial infarction: An independent study of Han population. Experimental gerontology. 2015; 72:230-8.
45. Park K-m, Teoh J-p, Wang Y, Broskova Z, Bayoumi A S, Tang Y, et al. Carvedilol-responsive microRNAs, miR-199a-3p and-214 protect cardiomyocytes from simulated ischemia-reperfusion injury. American Journal of Physiology-Heart and Circulatory Physiology. 2016; 311 (2):H371-H83.
46. Su Y, Yuan J, Zhang F, Lei Q, Zhang T, Li K, et al. MicroRNA-181a-5p and microRNA-181a-3p cooperatively restrict vascular inflammation and atherosclerosis. Cell death & disease. 2019; 10(5):1-15.
47. Melman Y F, Shah R, Danielson K, Xiao J, Simonson B, Barth A, et al. Circulating MicroRNA-30d is associated with response to cardiac resynchronization therapy in heart failure and regulates cardiomyocyte apoptosis: a translational pilot study. Circulation.
48. Li J, Salvador A M, Li G, Valkov N, Ziegler O, Yeri A, et al. Mir-30d regulates cardiac remodeling by intracellular and paracrine signaling. Circulation Research. 2021; 128 (1):e1-e23.
49. Demolli S, Doebele C, Doddaballapur A, Lang V, Fisslthaler B, Chavakis E, et al. MicroRNA-30 mediates anti-inflammatory effects of shear stress and KLF2 via repression of angiopoietin 2. Journal of molecular and cellular cardiology. 2015; 88:111-9.
50. Nascimento Conde J, Schutt W R, Gorbunova E E, and Mackow E R. Recombinant ACE2 Expression Is Required for SARS-CoV-2 To Infect Primary Human Endothelial Cells and Induce Inflammatory and Procoagulative Responses. mBio. 2020; 11 (6).
51. Perez-Bermejo J A, Kang S, Rockwood S J, Simoneau C R, Joy D A, Silva A C, et al. SARS-CoV-2 infection of human iPSC—derived cardiac cells reflects cytopathic features in hearts of patients with COV ID-19. Science Translational Medicine. 2021; 13 (590):eabf7872.
52. Goldenberg N M, Steinberg B E, Slutsky A S, and Lee W L. Broken barriers: a new take on sepsis pathogenesis. Sci Transl Med. 2011; 3(88):88ps25.
53. Monteil V, Kwon H, Prado P, Hagelkruys A, Wimmer R A, Stahl M, et al. Inhibition of SARS-CoV-2 Infections in Engineered Human Tissues Using Clinical-Grade Soluble Human ACE2. Cell. 2020; 181(4):905-13.e7.
54. Gavard J, and Gutkind J S. V E-cadherin and claudin-5: it takes two to tango. Nature Cell Biology. 2008; 10(8):883-5.
55. Jones C A, Nishiya N, London N R, Zhu W, Sorensen L K, Chan A C, et al. Slit2-Robo4 signalling promotes vascular stability by blocking Arf6 activity. Nat Cell Biol. 2009; 11(11):1325-31.
56. Gibot S, Jolly L, Lemarié J, Carrasco K, Derive M, and Boufenzer A. Triggering receptor expressed on myeloid cells-1 inhibitor targeted to endothelium decreases cell activation. Frontiers in immunology. 2019; 10:2314.
57. Romero I A, Radewicz K, Jubin E, Michel C C, Greenwood J, Couraud P-O, et al. Changes in cytoskeletal and tight junctional proteins correlate with decreased permeability induced by dexamethasone in cultured rat brain endothelial cells. Neuroscience letters. 2003; 344(2):112-6.
58. Leisman D E, Deutschman C S, and Legrand M. Facing COVID-19 in the ICU: vascular dysfunction, thrombosis, and dysregulated inflammation. Intensive care medicine. 2020; 46(6):1105-8.
59. Dupont A, Rauch A, Staessens S, Moussa M, Rosa M, Corseaux D, et al. Vascular endothelial damage in the pathogenesis of organ injury in severe COVID-19. Arteriosclerosis, Thrombosis, and Vascular Biology. 2021; 41(5):1760-73.
60. Bouhaddou M, Memon D, Meyer B, White K M, Rezelj V V, Marrero M C, et al. The global phosphorylation landscape of SARS-CoV-2 infection. Cell. 2020; 182(3):685-712. e19.
61. Stephenson E, Reynolds G, Botting R A, Calero-Nieto F J, Morgan M D, Tuong Z K, et al. Single-cell multi-omics analysis of the immune response in COVID-19. Nature Medicine. 2021; 27(5):904-16.
62. Ren X, Wen W, Fan X, Hou W, Su B, Cai P, et al. COVID-19 immune features revealed by a large-scale single-cell transcriptome atlas. Cell. 2021; 184(7):1895-913. e19.
63. Li S, Jiang L, Li X, Lin F, Wang Y, Li B, et al. Clinical and pathological investigation of patients with severe COVID-19. JCI Insight. 2020; 5 (12).
64. Han Y, Li Y, and Jiang Y. The Prognostic Value of Plasma MicroRNA-155 and MicroRNA-146a Level in Severe Sepsis and Sepsis-Induced Acute Lung Injury Patients. Clin Lab. 2016; 62(12):2355-60.
65. Shen X, Zhang J, Huang Y, Tong J, Zhang L, Zhang Z, et al. Accuracy of circulating microRNAs in diagnosis of sepsis: a systematic review and meta-analysis. Journal of Intensive Care. 2020; 8(1):84.
66. Roberts J D, Wells G A, Le May M R, Labinaz M, Glover C, Froeschl M, et al. Point-of-care genetic testing for personalisation of antiplatelet treatment (RAPID GENE): a prospective, randomised, proof-of-concept trial. The Lancet. 2012; 379(9827):1705-11.
67. Dave V P, Ngo T A, Pernestig A-K, Tilevik D, Kant K, Nguyen T, et al. MicroRNA amplification and detection technologies: opportunities and challenges for point of care diagnostics. Laboratory Investigation. 2019; 99(4):452-69.
68. Boštjančič E, Zidar N, Štajer D, and Glavač D. MicroRNAs miR-1, miR-133a, miR-133b and miR-208 are dysregulated in human myocardial infarction. Cardiology. 2010; 115(3):163-9.
69. Pons S, Arnaud M, Loiselle M, Arrii E, Azoulay E, and Zafrani L. Immune consequences of endothelial cells' activation and dysfunction during sepsis. Critical care clinics. 2020; 36(2):401-13.
70. Lee W L, and Slutsky A S. Sepsis and endothelial permeability. N Engl J Med. 2010; 363(7):689-91.
71. Thickett D R, Armstrong L, Christie S J, and Millar A B. Vascular endothelial growth factor may contribute to increased vascular permeability in acute respiratory distress syndrome. Am J Respir Crit Care Med. 2001; 164(9):1601-5.
72. Xiong S, Zhang L, Richner J M, Class J, Rehman J, and Malik A B. Interleukin-1RA Mitigates SARS-CoV-2-Induced Inflammatory Lung Vascular Leakage and Mortality in Humanized K18-hACE-2 Mice. Arterioscler Thromb Vasc Biol. 2021:Atvbaha121316925.
73. Hadjadj J, Yatim N, Barnabei L, Corneau A, Boussier J, Smith N, et al. Impaired type I interferon activity and inflammatory responses in severe COVID-19 patients. Science. 2020; 369(6504):718-24.
74. Mills R J, Humphrey S J, Fortuna P R J, Lor M, Foster S R, Quaife-Ryan G A, et al. BET inhibition blocks inflammation-induced cardiac dysfunction and SARS-CoV-2 infection. Cell. 2021; 184(8):2167-82.e22.
75. Alsaffar H, Martino N, Garrett J P, and Adam A P. Interleukin-6 promotes a sustained loss of endothelial barrier function via Janus kinase-mediated STAT3 phosphorylation and de novo protein synthesis. Am J Physiol Cell Physiol. 2018; 314 (5):C589-c602.
76. Wang S, Le T Q, Kurihara N, Chida J, Cisse Y, Yano M, et al. Influenza Virus— cytokine-protease cycle in the pathogenesis of vascular hyperpermeability in severe influenza. The Journal of infectious diseases. 2010; 202(7):991-1001.
77. London N R, Zhu W, Bozza F A, Smith M C P, Greif D M, Sorensen L K, et al. Targeting Robo4-dependent Slit signaling to survive the cytokine storm in sepsis and influenza. Science translational medicine. 2010; 2(23):23ra19-23ra19.
78. Xiao F, Wang D, Kong L, Li M, Feng Z, Shuai B, et al. Intermedin protects against sepsis by concurrently re-establishing the endothelial barrier and alleviating inflammatory responses. Nature Communications. 2018; 9(1):2644.
79. Higgins S J, Purcell L A, Silver K L, Tran V, Crowley V, Hawkes M, et al. Dysregulation of angiopoietin-1 plays a mechanistic role in the pathogenesis of cerebral malaria. Science translational medicine. 2016; 8(358):358ra128-358ra128.
80. Geven C, Blet A, Kox M, Hartmann O, Scigalla P, Zimmermann J, et al. A double-blind, placebo-controlled, randomised, multicentre, proof-of-concept and dose-finding phase II clinical trial to investigate the safety, tolerability and efficacy of adrecizumab in patients with septic shock and elevated adrenomedullin concentration (AdrenOSS-2). BMJ Open. 2019; 9 (2):e024475.
81. Hippenstiel S, Witzenrath M, Schmeck B, Hocke A, Krisp M, Krüll M, et al. Adrenomedullin reduces endothelial hyperpermeability. Circulation research. 2002; 91(7):618-25.

Primary Figure Legends

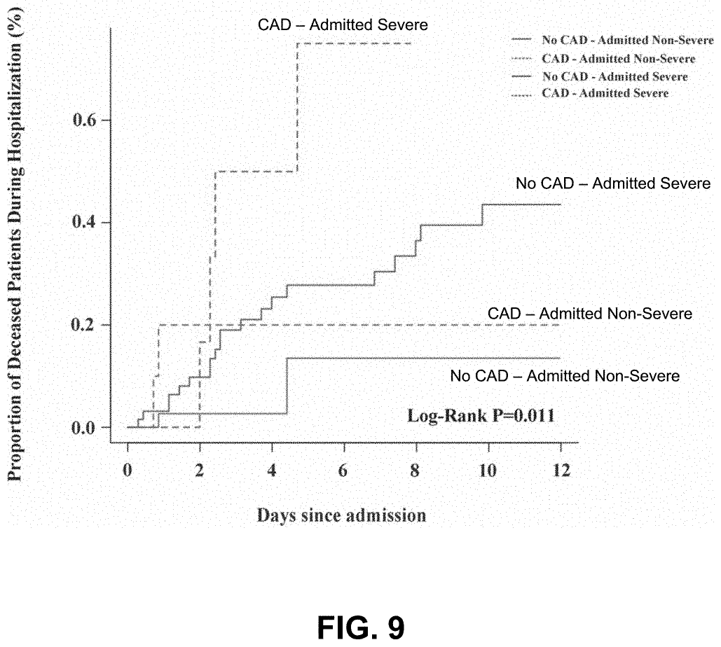

FIG. 1. Unadjusted Kaplan-Meier Estimates of Survival. (a) Log-rank test comparing curves of all admitted patients with severe COVID-19 to all admitted non-severe COVID-19 patients (i.e., mild and moderate vs severe, log-rank, P<0.001). (b) Log-rank test comparing curves of those with severe COVID-19 to severity matched SARS-CoV-2 negative patients (P=0.42). Refer to FIG. 9 as well as Tables IV-VI for more information.

FIG. 2: Plasma Concentration of Endothelial Dysfunction and Inflammatory Markers at T₀₋₁. (a) Levels of Ang-2 and (b) sVCAM-1 stratified among disease severity. Data shown are for all patients with an available t₀₋₁ sample (n=210), with values representing the mean and error bars are (±S.D.). (c) Unadjusted log hazard ratio of Ang-2 and (d) sVCAM-1 in severe disease patients (n=76) with univariable p-value in association with mortality. P values for multiple group comparisons were determined by Kruskal-Wallis test with Dunn's multiple comparisons test. Severe negative statistical comparisons are only shown in reference to the concordant severe group. Red hash marks indicate individual samples. Abbreviations: Ang-2, Angiopoietin-2; VCAM-1, Vascular cell adhesion molecule 1; N.S., non-significant. Refer to FIG. 10-19 for more information and additional endothelial and inflammatory markers.

FIG. 3: Plasma MiRNA Transcriptome Across the COVID-19 Severity. (a) Pie chart percent modulation of the transcriptome for all subgroups studied. Volcano plots of differentially expressed miRNA between patient groups (b, d) with predicted KEGG terms (with enrichment score below and number of genes to the right) for pathways of deregulated miRNAs shown beside each corresponding region of the volcano plot (c, e). Data are displayed as false discovery rate (FDR) adjusted P values (Q values) vs the log₂ fold change, with dashed lines drawn to define restriction boundaries. Refer to FIG. 20-21 for more information.

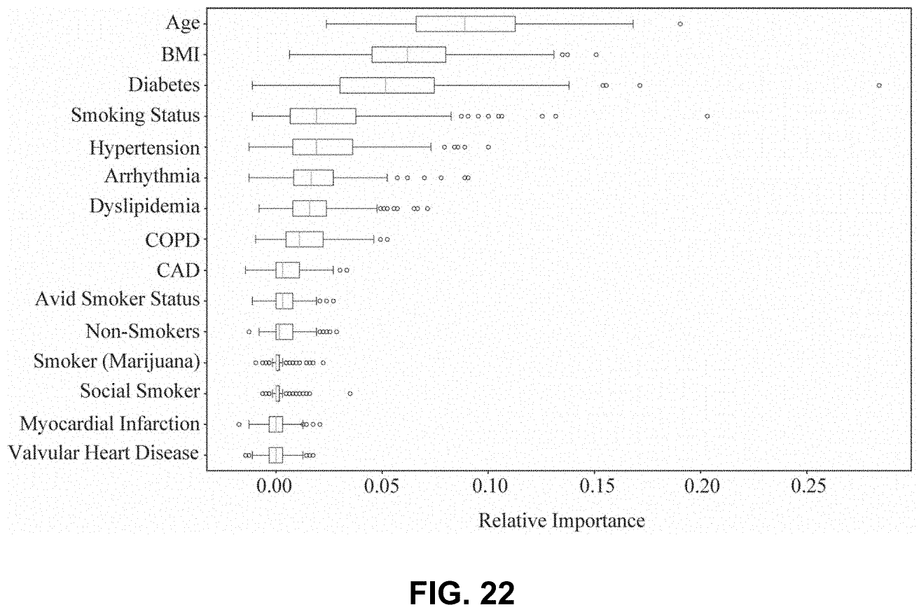

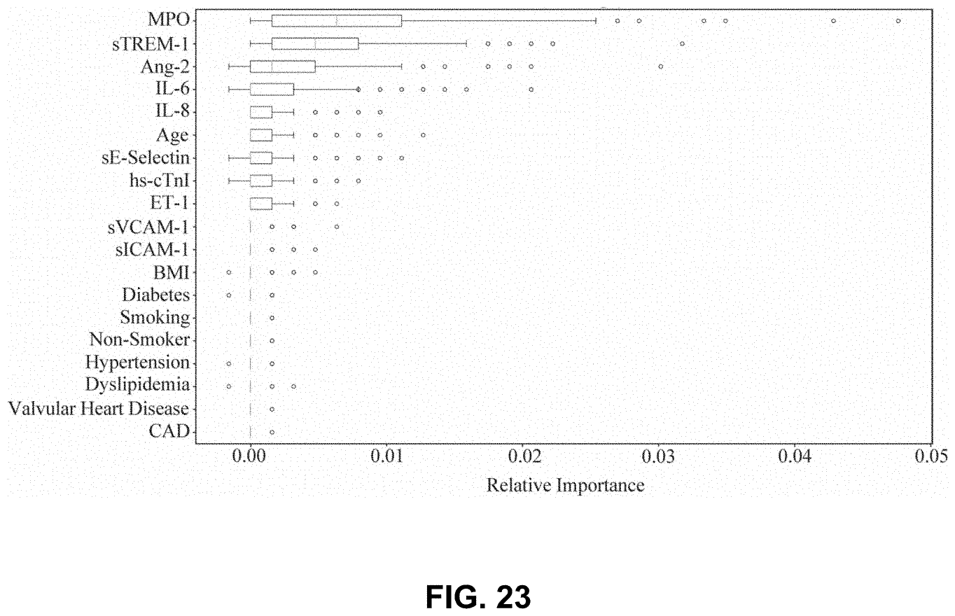

FIG. 4: Machine Learning Approach to Risk Assessment and Association of Biomarkers with In-Hospital Mortality for Severe COVID-19 Patients. Assessment of datasets using repeated randomized stratified sub-sampling cross-validation (Random Forest machine learning) for (a) clinical data, (b) clinical data and protein expression metrics, and (c) clinical data and miRNA atlas expression metrics. Univariable log hazard ratios of candidate microRNAs (d) hsa-miR-6080, (e) hsa-miR-30e-5p, (f) hsa-miR-30c-5p, and (g) hsa-miR-30b-5p in relation to mortality. Red hash marks indicate individual samples. Refer to FIG. 22-23 for more information.

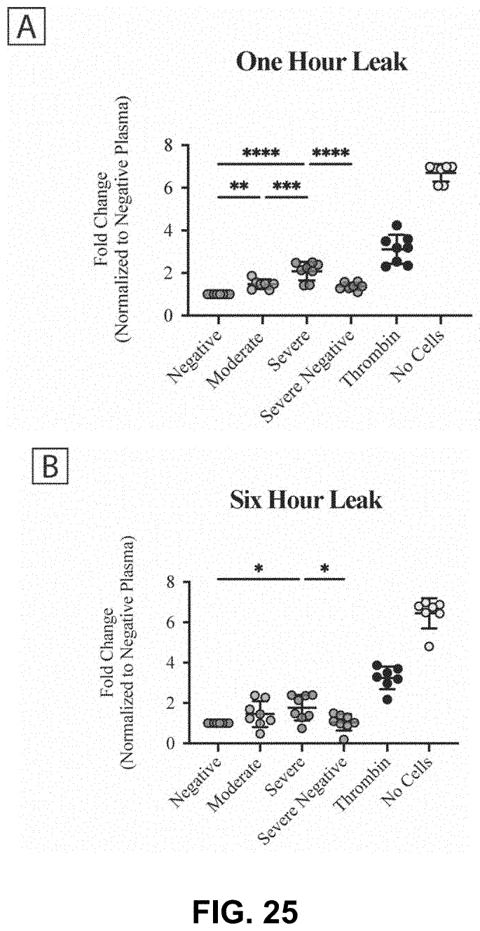

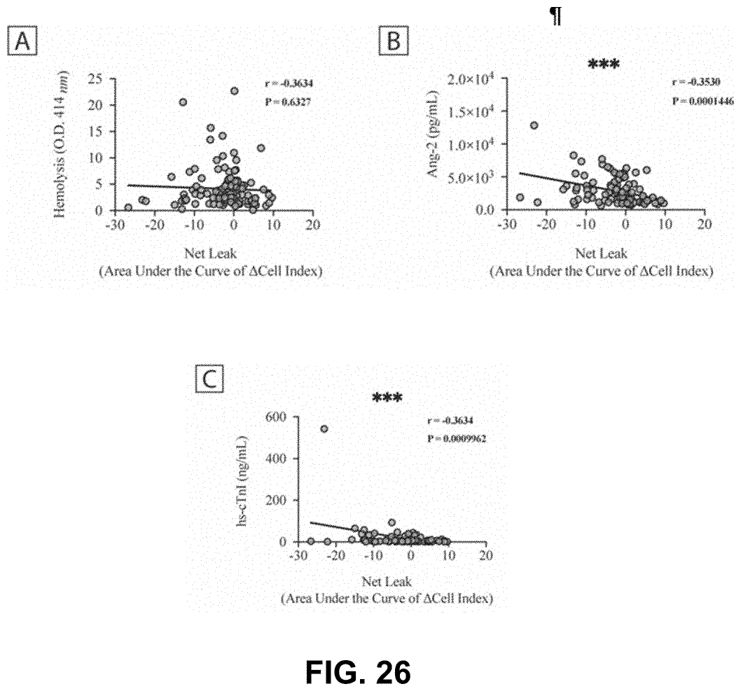

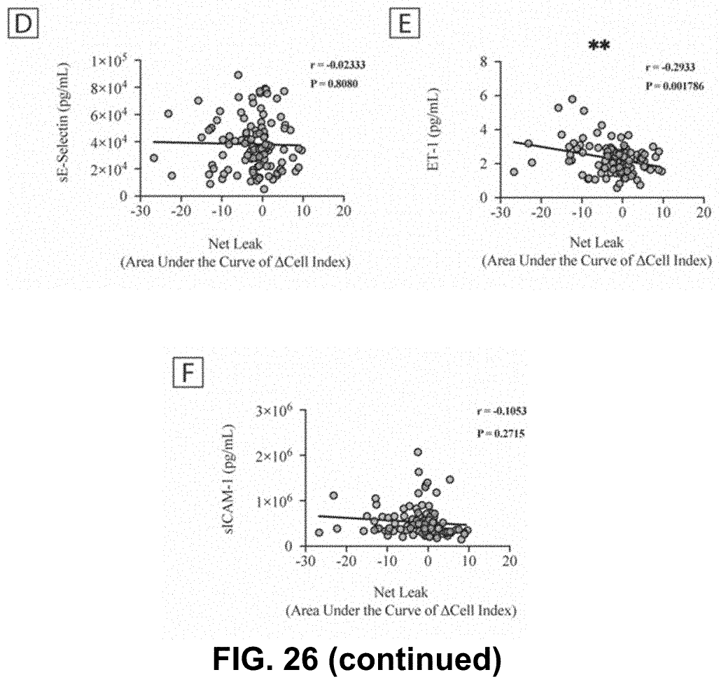

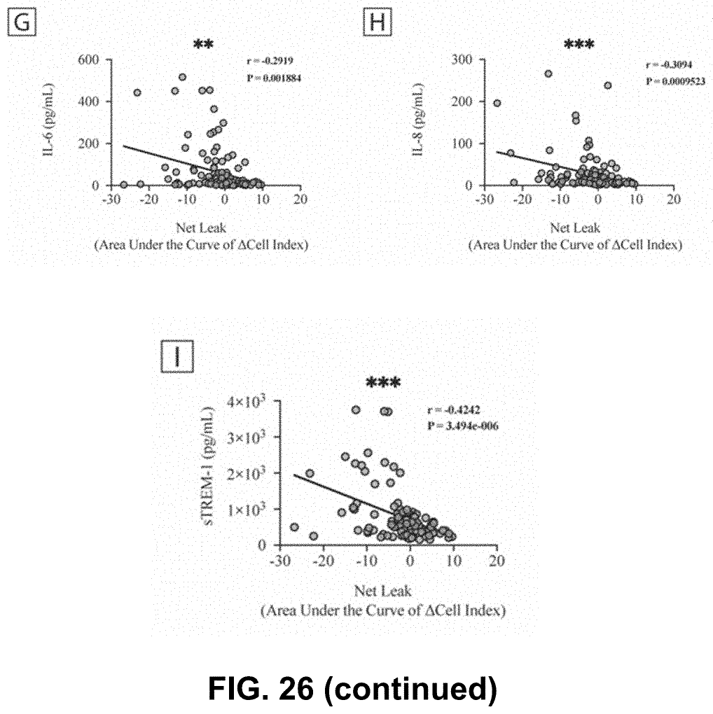

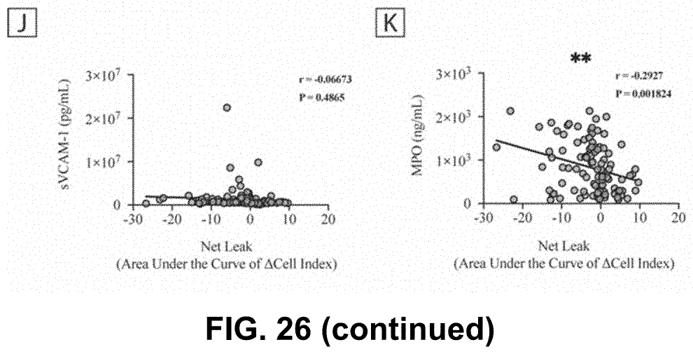

FIG. 5: Endothelial Barrier Disruption Driven by Modulation of Inflammatory and Cytoskeletal Pathways is Disease Severity Dependent. (a) Overview of parallel endothelial phenotype monitoring. The overall scheme is shown, representing the real-time monitoring of EC barrier function (xCelligence) followed by both mRNA sequencing and multiplexed immunohistochemistry. (b) Time-course monitoring of pHUVEC barrier function during coincubation with 20% (v/v) plasma (left) sampled from t₀₋₁ (negative, n=30; mild, n=14; moderate disease, n=15; severe disease, n=52; thrombin, n=7) compared to ‘mock’ treatment (i.e., PBS control) and quantification of area under the curve across the six-hour coincubation period (right). Thrombin treatment was included as a barrier disrupting positive control. Barrier data displayed depicts t₀₋₁ adjusted values (media only) that were subsequently normalized to mock-treated cells (PBS, dashed line) and displayed as the mean of the change in cell index from experiment initiation. Note: some error bars are too small to be visible on the figure. Quantified values are relative to ‘mock’ treatment and represent mean and error bars (±S.D.). P values determined by a Kruskal-Wallis one-way analysis of variance with Dunn's multiple comparison correction. moderate vs negative, p=3.1×10⁻³; severe vs negative, p=4.7×10⁻⁵. (c) Principal component analysis plot, with the 2D coordinates of each profile based on the scores of the first two principal components. (d) Volcano plot displaying the −log₁₀ of the adjusted P values vs the log₂ fold change of respective disease severities compared with COVID-19-negative control transcript expression. Red and blue markers indicate adjusted FDR-adjusted P values <0.05 for up- and down-regulation, respectively, based on a log fold-change of >±0.58. (e) Gene set enrichment plot of the top-ranked gene set, TNF Targets Up (FDR=7.05×10⁻⁹, NES=3.13), WT1 Targets Up (FDR=1.45×10⁻⁸, NES=2.66), and interferon responsive genes (FDR=4.52×10⁻⁹, NES=3.24) using all genes ranked by their magnitude of association with each respective disease severity group (the enrichment P value shown was computed from the GSEA test) along with top ranked gene sets (below). The tick marks denote the location of the genes in each respective module. Fold change of all genes between the compared conditions are shown as bar plots in the bottom panels (x axis: genes ranked by −log₂ fold change; y axis: −log₁₀ fold change). (f) Top ten significantly enriched pathways based on all genes ranked by fold change identified by gProfiler are shown for each comparison. Data Files containing a full list of differentially expressed genes, GSEAs, and pathways are available (raw data not presented herein). Refer to FIG. 25-26 for more information.

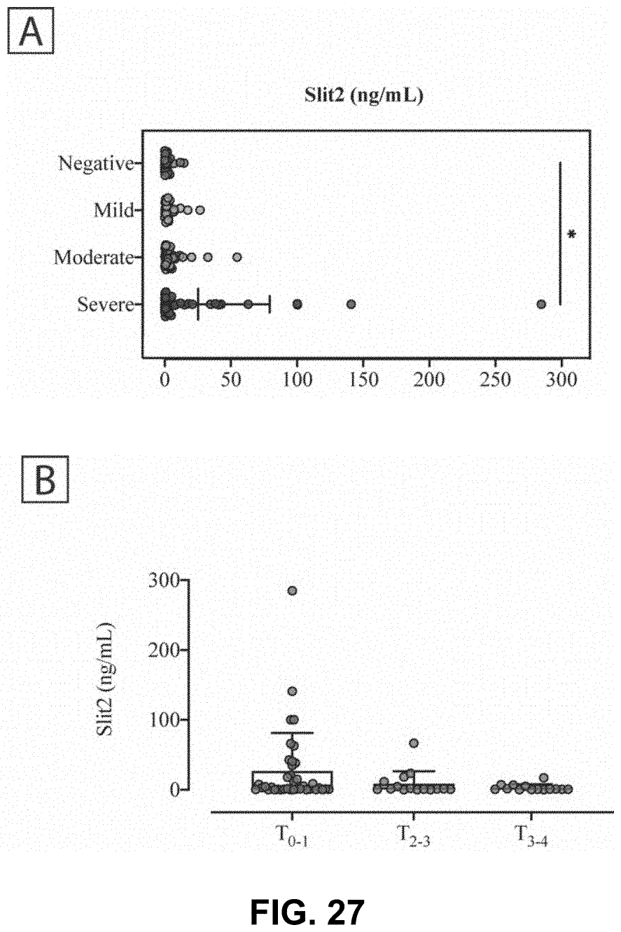

FIG. 6. Targeted Modulators of Endothelial Barrier Protect Against COVID-19 Plasma-Induced Endothelial Barrier Dysfunction. (a) Left; Representative confocal microscopy images and brightness enhanced zooms depicting VE-cadherin (red) and Claudin-5 (green) junctional staining after six hours of treatment of pHUVECs with t₀₋₁ COVID-19 plasma and matched controls; n=3-4 per group. Scale bars: 50 μm. Right; Quantification of VE-cadherin and Claudin-5 pixel intensity for each respective group. Each dot represents mean protein expression across at least four representative sections of a biological replicate. Center bars represent mean and error bars are (±S.D.). P values were determined by one-way ANOVA with Tukey's multiple comparisons test. Mild vs negative: VE-cadherin, P=6.0×10⁻³. Moderate vs. negative: VE-cadherin, P=4.0×10⁻⁴; Claudin-5, p=1.6×10⁻². Negative vs severe: VE-cadherin, P=1.6×10⁻³; Claudin-5, P=8.0×10⁻³. Mild vs moderate: Claudin-5, P=2.9×10⁻². Mild vs severe: Claudin-5, P=1.4×10⁻². (b-e) Left; Representative confocal microscopy images depicting a combinatorial screen of COVID-19 plasma and matched controls treated with either (b) Slit2-N, (c) Nangibotide, and (d) Dexamethasone on pHUVECs reveals modulated maintenance of VE-cadherin (red) and Claudin-5 (green) expression; n=3-4 per group. Scale bars: 50 μm. Middle; Quantification of VE-cadherin and claudin-5 for each respective group. Imaging P values were determined by one-way ANOVA with Dunnett's multiple comparisons test (all compared to control). Nangibotide; Moderate vs negative: VE-cadherin *P=1.3×10⁻², Claudin-5 **P=8.2×10⁻³. Dexamethasone; Severe vs negative: VE-cadherin *P=2.4×10⁻², Claudin-5 *P=3.3×10⁻³. Right; Quantification of VE-cadherin and Claudin-5 for each respective group. Each dot represents mean protein expression across at least four representative sections of a biological replicate. Center bars represent mean and error bars are (±S.D.). Right; TEER quantification of corresponding treatment groups. P values were determined by Kruskal-Wallis test with Dunn's multiple comparisons test (all compared to control). Nangibotide; Negative vs moderate (P): *P=3.9×10⁻²; Severe vs negative ****P=4.3×10⁻⁵. Dexamethasone; Moderate vs negative (P): **P=6.2×10⁻³; Severe vs negative****P=2.2×10⁻⁵. Refer to FIG. 27 for more information.

Primary Tables

TABLE 1
Patient Demographics and Clinical Characteristics of Patients at T₀₋₁

| | Disease Severity | | | | | |
|---|---|---|---|---|---|---|
| | Mild | | | | Severe | |
| | COVID-19 | | | | COVID-19 | |
| | Negative | Mild | Moderate | Severe | Negative | |
| Characteristics* | (n = 30) | (n = 27) | (n = 39) | (n = 76) | (n = 69) | P-value |

Demographics

| | | | | | | | | | | | |
|---|---|---|---|---|---|---|---|---|---|---|---|
| Age, median (IQR) - yr. | 47.0 | (33.0-60.5) | 59.0 | (41.0-70.0) | 74.0 | (59.0-86.0) | 61.0 | (52.0-71.0) | 61.0 | (51.0-72.0) | <0.0001 |
| Distribution - no. (%) | | | | | | | | | | | |
| 18-40 yr. | 11 | (36.7) | 6 | (22.2) | 4 | (10.3) | 2 | (2.6) | 11 | (15.9) | <0.0001 |
| 41-64 yr. | 13 | (43.3) | 9 | (33.3) | 9 | (23.1) | 45 | (59.2) | 26 | (37.7) | 0.0025 |
| ≥65 yr. | 6 | (20.0) | 12 | (44.4) | 26 | (66.7) | 29 | (38.2) | 32 | (46.4) | 0.0023 |
| Male Sex, no./total no. (%) | 17/30 | (56.7) | 15/27 | (55.6) | 25/39 | (64.1) | 52/76 | (68.4) | 46/69 | (66.7) | 0.6564 |
| BMI, median (IQR)^{†} | 25.2 | (21.3-32.6) | 25.9 | (22.5-35.0) | 26.0 | (21.1-30.0) | 28.1 | (24.2-31.8) | 25.7 | (21.8-32.5) | 0.4798 |
| Obesity^{‡}, no./total no. (%) | 4/19 | (21.1) | 6/18 | (33.3) | 6/27 | (22.2) | 38/61 | (62.3) | 35/69 | (50.7) | 0.0006 |
| Smoking History - | | | | | | | | | | | |
| no./total no. (%)^{§} | | | | | | | | | | | |
| Current smoker | 4/12 | (33.3) | 3/9 | (33.3) | 3/5 | (60.0) | 2/43 | (4.7) | 12/69 | (17.4) | 0.0027 |
| Previous smoker | 5/12 | (41.7) | 0/9 | (0.0) | 2/5 | (40.0) | 5/43 | (11.6) | 4/69 | (5.8) | 0.0032 |
| Never smoked | 3/12 | (25.0) | 6/9 | (66.7) | 0/5 | (0.0) | 36/43 | (83.7) | 53/69 | (76.8) | <0.0001 |
| Length of Admission - | 0 | (0-2.3) | 2 | (0-13) | 12 | (7-24) | 28 | (16-65) | 14 | (7.5-28) | <0.0001 |
| days (IQR) | | | | | | | | | | | |

| | | | | | | | | | | |
|---|---|---|---|---|---|---|---|---|---|---|
| Mortality | — | 3/27 | (11.1) | 1/39 | (2.6) | 29/76 | (38.2) | 21/69 | (30.4) | <0.0001 |

Co-Existing Disorder - no./total no. (%)

| | | | | | | | | | | | |
|---|---|---|---|---|---|---|---|---|---|---|---|
| Arrythmia | 3/30 | (10.0) | 3/27 | (11.1) | 9/39 | (23.1) | 10/69 | (14.5) | 12/69 | (17.4) | 0.6140 |
| Asthma | 1/30 | (3.3) | 1/27 | (3.7) | 2/39 | (5.1) | 8/70 | (11.4) | 3/69 | (4.3) | 0.4970 |
| Cardiac procedure | 2/30 | (6.7) | 2/27 | (7.4) | 10/39 | (25.6) | 6/69 | (8.7) | 7/69 | (10.1) | 0.9740 |
| Chronic kidney disease | 1/30 | (3.3) | 1/27 | (3.7) | 13/39 | (33.3) | 10/70 | (14.3) | 6/69 | (87.0) | 0.0204 |
| Congestive heart failure | 3/30 | (10.0) | 3/27 | (11.1) | 5/39 | (12.8) | 5/69 | (7.2) | 10/69 | (14.5) | 0.7389 |
| COPD | 3/30 | (10.0) | 5/27 | (18.5) | 5/39 | (12.8) | 6/69 | (8.7) | 14/69 | (20.3) | 0.3134 |
| Coronary artery disease | 6/30 | (20.0) | 5/27 | (18.5) | 8/39 | (20.5) | 6/69 | (8.7) | 8/69 | (11.6) | 0.2802 |
| Diabetes mellitus | 2/30 | (6.7) | 11/27 | (40.7) | 16/39 | (41.0) | 35/69 | (50.7) | 19/69 | (27.5) | <0.0001 |
| Dyslipidemia | 5/30 | (16.7) | 6/27 | (22.2) | 19/39 | (48.7) | 22/69 | (31.9) | 16/69 | (23.2) | 0.0266 |
| Gastroesophageal reflux | 0/30 | (0.0) | 1/27 | (3.7) | 0/39 | (0.0) | 10/69 | (14.5) | 5/69 | (7.2) | 0.0019 |
| disease | | | | | | | | | | | |
| Gout | 0/30 | (0.0) | 1/27 | (3.7) | 0/39 | (0.0) | 2/70 | (2.9) | 0/69 | (0.0) | 0.2165 |
| Hypertension | 6/30 | (20.0) | 12/27 | (44.4) | 20/39 | (51.3) | 40/69 | (58.0) | 30/69 | (43.5) | 0.0105 |
| Immunocompromised | 2/30 | (6.7) | 1/27 | (3.7) | 1/39 | (2.6) | 1/70 | (1.4) | 2/69 | (2.9) | 0.6173 |
| Malignancy | | | | | | | | | | | |
| None | 29/30 | (96.7) | 22/27 | (81.5) | 33/39 | (84.6) | 65/70 | (92.9) | 63/69 | (91.3) | 0.2279 |
| Active | 0/30 | (0.0) | 3/27 | (11.1) | 2/39 | (5.1) | 2/70 | (2.9) | 2/69 | (2.9) | 0.2605 |
| Previous | 1/30 | (3.3) | 2/27 | (7.4) | 4/39 | (10.3) | 3/70 | (4.3) | 4/69 | (5.8) | 0.7546 |
| Myocardial infarction | 3/30 | (10.0) | 2/27 | (7.4) | 3/39 | (7.7) | 5/69 | (7.2) | 4/69 | (5.8) | 0.9505 |
| Obstructive sleep apnea | 2/30 | (6.7) | 3/27 | (11.1) | 4/39 | (10.3) | 8/70 | (11.4) | 5/69 | (7.2) | 0.9038 |
| Non-COVID-19 | 0/30 | (0.0) | 0/27 | (0.0) | 0/39 | (0.0) | 2/70 | (2.9) | 2/69 | (2.9) | 0.8958 |
| Pneumonia | | | | | | | | | | | |
| Peripheral vascular | 1/30 | (3.3) | 2/27 | (7.4) | 1/39 | (2.6) | 3/70 | (4.3) | 3/69 | (4.3) | 0.9263 |
| disease | | | | | | | | | | | |
| Stroke | 0/30 | (0.0) | 0/27 | (0.0) | 0/39 | (0.0) | 3/70 | (4.3) | 0/69 | (0.0) | 0.3294 |

Concurrent Therapeutics - no./total no. (%)

| | | | | | | | | | | | |
|---|---|---|---|---|---|---|---|---|---|---|---|
| Angiotensin II receptor | 1/30 | (3.3) | 4/26 | (15.4) | 2/39 | (5.1) | 3/64 | (4.7) | 1/68 | (1.5) | 0.1054 |
| blockers | | | | | | | | | | | |
| ACE inhibitor | 1/30 | (3.3) | 2/26 | (7.7) | 2/39 | (5.1) | 14/64 | (21.9) | 7/68 | (10.3) | 0.1007 |
| Anticoagulant | 7/30 | (23.3) | 5/26 | (19.2) | 4/39 | (10.3) | 6/64 | (9.4) | 7/68 | (10.3) | 0.2749 |
| Beta blocker | 4/30 | (13.3) | 4/26 | (15.4) | 15/39 | (38.5) | 16/64 | (25.0) | 12/68 | (17.6) | 0.0781 |
| Calcium channel blocker | 3/30 | (10.0) | 4/26 | (15.4) | 6/39 | (15.4) | 16/64 | (25.0) | 4/68 | (5.9) | 0.0338 |
| Diuretics | 1/30 | (3.3) | 1/26 | (3.8) | 5/39 | (12.8) | 14/64 | (21.9) | 18/68 | (26.5) | 0.0088 |
| Statin | 3/30 | (10.0) | 7/26 | (26.9) | 18/39 | (46.2) | 21/64 | (32.8) | 20/68 | (29.4) | 0.2992 |

*Summary statistics are based on the full population indicated in the column heading.
^{†}Body mass index is the weight in kilograms divided by the square of the height in meters.
^{‡}Obesity is classified according to World Health Organization guidelines (i.e., >25).
^{§}Smoker is defined as an aggregate of self-reported current and previous smokers.
Abbreviations: ACE = Angiotensin-converting-enzyme inhibitors; COPD = Chronic obstructive pulmonary disease; BMI = Body Mass Index; IQR = Interquartile range. Percentages may not add up to 100% due to rounding.

Supplemental Section

Supplemental Abbreviations and Acronyms

| | |
|---|---|
| ACE | Angiotensin-converting-enzyme inhibitors |
| Ang-2 | Angiopoietin-2 |
| APACHE | Acute physiologic assessment and chronic health evaluation |
| ARBs | Angiotensin II receptor blockers |
| ARDS | Acute respiratory distress syndrome |
| BMI | Body mass index |
| CAD | Coronary artery disease |
| Cat# | Catalogue number |
| CCB | Calcium channel blocker |
| CF | Cystic fibrosis |
| CKD | Chronic kidney disease |
| COPD | Chronic obstructive pulmonary disease |
| COVID-19 | Coronavirus Disease 2019 |
| CV | Cardiovascular |
| DAPI | 4′,6-diamidino-2-phenylindole |
| EBM | Endothelial basal media |
| ECMO | Extracorporeal membrane oxygenation |
| ED | Emergency department |
| EGM | Endothelial growth medium |
| ET-1 | Endothelin-1 |
| FDR | False discovery rate |
| FITC | Fluorescein isothiocyanate |
| FiO₂ | Fraction of inspired oxygen |
| GERD | Gastroesophageal reflux disease |
| GSEA | Gene set enrichment analysis |
| GSE | Gene expression omnibus series accession number |
| Hs-cTnI | High-sensitivity cardiac troponin |
| Ig | Immunoglobulin |
| IL | Interleukin |
| IQR | Interquartile range |
| kDa | Kilodalton |
| KEGG | Kyoto encyclopedia of genes and genomes |
| miR/miRNA | MicroRNA |
| MIV | Mechanical ventilation |
| MPO | Myeloperoxidase |
| NIV | Non-invasive ventilation |
| NSTEMI | Non-ST-elevation myocardial infarction |
| OSA | Obstructive sleep apnea |
| PAD | Peripheral artery disease |
| PF | Ratio of arterial oxygen partial pressure to fractional inspired oxygen |
| PFO | Patent foramen ovale |
| pHUVEC | Pooled human umbilical vein endothelial cells |
| QC | Quality control |
| REB | Research ethics board |
| RR | Respiratory rate |
| RRID:AB | Research resource identifier (antibody) |
| RTCA | Real-time cell analyzer |
| S.D. | Standard deviation |
| SARS-COV-2 | Severe acute respiratory syndrome coronavirus-2 |
| sICAM-1 | Soluble intercellular adhesion molecule-1 |
| SOFA | Sequential organ failure assessment |
| sSLIT2 | Soluble slit guidance ligand 2 |
| sTREM-1 | Soluble triggering receptor expressed on myeloid cells-1 |
| sVCAM-1 | Soluble vascular cell adhesion molecule-1 |
| TIA | Transient ischemia attack |
| TC | Tissue culture |
| TEER | Transendothelial electrical resistance |
| TIA | Transient ischemic attack |
| TNFα | Tumor necrosis factor alpha |
| VTE | Venous thromboembolism |
| WBCs | White blood cell |

Supplemental Methods

Rationale and Design: Coronavirus Disease 2019 (COVID-19) is a leading infectious etiology currently present throughout the world, for which there are limited therapeutic interventions. Understanding the pathobiology of COVID-19 outcomes can enable personalized patient management protocols, improve survival, and aid in the preparation of diagnostic tools for future pandemics. Due to the dynamic nature of clinical care guidelines, clinical care did not follow a standardized protocol but rather was determined by individual providers and patient needs. Plasma of enrolled patients was taken at presentation and at set intervals (days 2-3 [t₂₋₃], 4-5 [t₄₋₅], and 6-7 [t₆₋₇]) following obtainment of consent. Comprehensive, integrated analysis of plasma transcriptome data was performed to prioritize COVID-19 outcome signals.

Patient Categorization: After retrospective clinical adjudications of medical records and triaging protocols, patients were assigned to pre-defined clinical groups, centered around the National Institutes of Health, ‘Clinical Spectrum of SARS-CoV-2 Infection’′, those being: (i) severe acute respiratory syndrome coronavirus (SARS-CoV-2) negative patients with mild acute respiratory disease, (ii) mild COVID-19, (iii) moderate COVID-19, (iv) severe COVID-19, and (v) SARS-CoV-2 negative patients requiring intensive care unit (ICU) level management for severe respiratory illness. These assignments were made solely on the basis of information available in the medical record and were blind to any novel biomarker data, which had not yet been generated. For biomarker studies, where appropriate, patients were matched for age, sex, body mass index (BMI), and co-existing conditions with non-infected controls as reference groups.

Study Population: This is a multicenter, secondary analysis of a prospectively recruited longitudinal cohort study enrolling consecutive patients with suspected SARS-CoV-2 infection who were referred to two Canadian quaternary care networks in Toronto, Canada from May 2020 to December 2020: University Health Network and St. Michael's Hospital. All participants who were 18 years of age or older, provided either direct written informed consent or were consented into the study by a lawfully entitled substitute decision-maker on behalf of a participant when lacking the capacity to make the decision. The study, and consenting, was conducted in accordance with protocols approved by the Research Ethics Board (REB) of the University Health Network (REB #: 20-5453.6; Cardiovascular Disease and Outcomes among Patients with SARS-CoV-2 Infection During Admission and Post-Discharge [The COVID study]) or St. Michael's Hospital (REB #: 20-078; COVID-19 Longitudinal Biomarkers in Lung Injury [COLOBILI]). SARS-CoV-2-negative patients with severe respiratory illness symptoms were enrolled within the COLOBILI study. Diagnoses of COVID-19 were confirmed through real-time reverse transcription-polymerase chain reaction assays of nasopharyngeal swabs according to the Public Health Ontario guidelines for SARS-CoV-2 testing.² The timeframe for recruitment largely excludes community level spread for the variants of concern (i.e., B.1.1.7 [Alpha], B.1.351 [Beta], B.1.617.2 [Delta], and P.1 [Gamma]), with the assumption that all patients harbored one of the predominant unmutated G strains (i.e., GR, GH, and GV).³ Samples were collected prior to the initiation of public vaccination programs in Ontario, with all patients assumed to be unvaccinated. Admitted patients were followed up after their COVID-19 diagnosis, with all causes of in-hospital mortality, complications, and therapeutic regimen ascertained until discharge. Patients' data were extracted from the in-hospital electronic medical records, de-identified, and assigned random identification numbers which were used throughout the project. Information on sex, age, and pertinent clinical parameters for the cohorts are provided (Table 1). The laboratory parameters were collected as reported by the individual centers, with standard international reference ranges applied to decide the cutoff point for abnormal levels.

Clinical Data Collection: Clinical characteristics, medical history, therapeutics administered during admission, complications, and outcomes were obtained and extracted from electronic medical records by clinical coordinators. Participant disease severity was quantified according to the National Institutes of Health Clinical Spectrum of SARS-CoV-2 infection which is largely based on respiratory parameters. Complete follow-up was available only for those requiring admission to hospital. Obesity was defined as BMI 30 kg/m² in line with World Health Organization guidelines.⁴ Preexisting cardiovascular disease (i.e., atrial fibrillation, arterial hypertension, coronary artery disease, dyslipidemia, diabetes mellitus, chronic obstructive pulmonary disease, or heart failure), myocardial infarction, and concurrent active malignancy were defined and recorded from the electronic medical records at the discretion of the clinical coordinators upon availability from previously recorded histories. Smoking was collected as a self-reported variable, with social smoking being defined as less than <4 times per week, and avid smoker being anything greater than that. Cardiovascular complications were defined as a surrogate of new-onset atrial or ventricular arrhythmia (atrial fibrillation, atrial flutter, non-sustained ventricular tachycardia, sustained ventricular tachycardia), acute coronary syndrome, clinical heart failure, right ventricular failure, left ventricular failure, biventricular failure, moderate or greater pulmonary embolism, tamponade, stroke, acute non-coronary ischemia due to hypercoagulable state, or intracardiac thrombus. In patients admitted to the ICU the Acute Physiologic Assessment and Chronic Health Evaluation II and the Sequential Organ Failure Assessment scores were used.^{5,6} These are validated methods for grading the severity of illness in critically ill patients based upon point-scoring systems associated with the degree of dysfunction with specific physiologic variables.

Processing of Bloodwork: Peripheral blood samples were collected synchronously with standard of care bloodwork at the University Health Network or St. Michael's Hospital between May 2020 and January 2021. Peripheral blood samples (10 mL) were drawn from the cubital vein into BD Vacutainer® Blood Collection Tubes (BD Bioscience, Franklin Lakes, N.J.) containing K₂EDTA and processed within three hours. Plasma was separated from whole blood through centrifugation (2,000×g, 24° C., 15 min) and stored at −80° C. until downstream processing. At no time during the process was the plasma subjected to temperatures below 4° C. or above 25° C. Samples were thawed on ice and the plasma was subjected to sequential centrifugation of (2,500×g, 4° C., 25 min) to reduce platelets and large particulate. Hemolysis was examined prior to downstream analysis by measuring the absorbance at 414 nm using a DS-11⁺Spectrophotometer (DeNovix, Wilmington, Del., United States).

Protein Biomarker Analysis: Circulating levels of angiopoietin-2 (Ang-2; Lower limit of quantification [LLOQ] 9.91 pg/mL), soluble CD62 antigen-like family member E (sE-Selectin; LLOQ 4.22 pg/mL), soluble CD54/intercellular adhesion molecule-1 (sICAM-1; LLOQ 4.1 pg/mL), soluble CD106/vascular cell adhesion protein-1 (sVCAM-1; LLOQ 137 pg/mL), CD105/endoglin, endothelin-1 (ET-1; LLOQ 0.250 pg/mL), interleukin-6 ([IL]-6; LLOQ 0.41 pg/mL), IL-8 (LLOQ 0.19 pg/mL), and soluble triggering receptor expressed on myeloid cells-1 (sTREM-1; LLOQ 4.19 pg/mL) were quantified in platelet free plasma samples using the Simple Plex Ella (ProteinSimple, San Jose, Calif., USA) multiplex platform according to the manufacturer's instructions; all Simple Plex values are reported as the average of triplicate readings. Soluble Slit homolog 2 protein (sSLIT2) was quantified using an enzyme-linked immunosorbent sandwich assay (ELISA; LLOQ 0.10 ng/mL, Elabscience, Wuhan, China). Myeloperoxidase was additionally quantified through ELISA (LLOQ; 0.062 ng/mL; R&D Systems Minneapolis, Minn., USA). Circulating cardiac troponin I (cTnI) was quantified using a CLIA certified high sensitivity ELISA kit (LLOQ 0.92 pg/mL; Biomatik, Kitchener, ON, CAN). Cardiac injury was defined as plasma levels of high-sensitivity cTnI (hs-cTnI) greater than the 99th percentile of normal values, as per clinical guidelines. See Table I for reagents.

Cell Culture: Pooled human umbilical vein endothelial cells ([pHUVECs], Lonza, Basel, Switzerland) from multiple donors were cultured in EC Growth Basal Medium-2 (Lonza, Basel, Switzerland) containing the complete EC Growth Medium BulletKit™ (Lonza, Basel, Switzerland) at 37° C. in a 5% CO₂ humidified incubator. Cells were maintained on 10 cm tissue culture plates (Corning, Costar, New York, N.Y., USA) coated with 0.1% (v/v) gelatin attachment factor (Thermo Fisher, Waltham, Mass., USA) and passaged every 2-4 days at 70%-80% confluency. For experimentation, pHUVECs at passages 3-7 were used. Cells tested negative for the presence of mycoplasma contamination (ThermoFisher, Waltham, Mass., USA).

xCelligence Real-Time Cell Analysis (Transendothelial Electrical Resistance [TEER]): Baseline resistance measurements were acquired using 150 μL EC Growth Basal Medium-2 for 15 minutes prior to experiment initiation using xCelligence Real-Time Cell Analyzer (RTCA, ACEA Biosciences, San Diego, Calif.). Following this, pHUVECs were seeded at densities of 4×10⁴/well into multiple 16-well E-plates (ACEA Biosciences, San Diego, Calif.) in volumes of 150 μL EC Growth Basal Medium-2 and monitored on the xCelligence RTCA. Once the cells reached confluency, 20% v/v plasma or 2 U/mL thrombin (Sigma-Aldrich, St Louis, Mo.) was added, and the cells incubated at 37° C. in a 5% carbon dioxide atmosphere for six hours. To quantify the change in permeability, cell indexes (surrogate metric for change in resistance across the monolayer) were adjusted to the resistance of reference wells (i.e., untreated cells in growth media) and normalized to the time point immediately prior to the addition of the plasma. The net area between the curve of the normalized dataset was utilized to calculate the overall change in permeability, with an increased area representing increases in TEER and decreases representing declines in TEER (i.e., loss of endothelial barrier function). During therapeutic testing, cells were co-treated with 1280 ng/mL recombinant Slit2-N⁸, 50 μg/mL nangibotide⁹, 3 μM dexamethasone¹⁰, or a matching DMSO control with Dulbecco's Phosphate Buffered Saline without magnesium and calcium ([PBS^{-/-}], Gibco, Gaithersburg, Md., USA.

Transwell Leak Assay: Endothelial monolayer leak assays were performed as previously described.¹¹ Briefly, pHUVECs were seeded on 3 μm pore transwell inserts (Corning Life Sciences, Corning, N.Y., USA) coated with 0.1% (v/v) gelatin attachment factor (ThermoFisher, Waltham, Mass., USA). The cells were grown on the inserts for two days until reaching 90%-95% confluency. Cells were subsequently treated for either one hour (acutely) or six hours (sub-chronically) with either 20% (v/v) human plasma or control PBS^{-/-}. Prior to leak quantification, cell media was changed to Hanks Buffered Salt Solution (ThermoFisher, Waltham, Mass., USA) and 1 mg/mL 40 kilodalton (kDa) fluorescein isothiocyanate—dextran (FITC-dextran, Sigma-Aldrich, St Louis, Mo.) was added to the top chamber with tracer flux subsequently being allowed to occur for one hour; 2 U/mL thrombin was added as a positive control to certain wells at the time of FITC addition. The experimenter was blinded to the grouping of each sample. The FITC accumulation in the bottom chamber was assessed in triplicate via excitation at 485 nm and emission at 535 nm using a Biotek Cytation 5 (Biotek, Winooski, Vt., USA).

Bulk RNA-Seq: Total RNA was isolated from treated pHUVEC samples (n=5 control [PBS^{-/-}], n=5 mild SARS-CoV-2 negative, n=5 mild disease, n=5 moderate disease, and n=5 severe disease) at the six-hour timepoint using the RNeasy Plus Micro kit (Qiagen, Germantown, Md., USA), after washing twice with ice-cold PBS^{-/-}. RNA quantities and quality were assessed using the Agilent 2100 Bioanalyzer (Agilent Technologies, Mississauga, ON, Canada). All samples passed a quality control threshold (RNA integrity number 7.0) to proceed to library preparations and RNA-seq. A total amount of 20 ng RNA per sample was used as input material for the RNA sample preparations. Sequencing libraries were generated using NEBNext® Ultra™ RNA Library Prep Kit for Illumina® (NEB, Ipswich, Mass., USA) following the manufacturer's recommendations and index codes were added to attribute sequences to each sample. Briefly, mRNA was purified from total RNA using poly-T oligo-attached magnetic beads. Fragmentation was carried out using divalent cations under elevated temperature in NEBNext First Strand Synthesis Reaction Buffer (5X). First-strand cDNA was synthesized using random hexamer primer and M-MuLV Reverse Transcriptase (RNase H−). Second strand cDNA synthesis was subsequently performed using DNA Polymerase I and RNase H. Remaining overhangs were converted into blunt ends via exonuclease/polymerase activities. After adenylation of 3′ ends of DNA fragments, NEBNext Adaptor with hairpin loop structure were ligated to prepare for hybridization. To select cDNA fragments of ˜150-200 bp in length, the library fragments were purified with AMPure XP system (Beckman Coulter, Beverly, USA). Then 3 μL USER Enzyme (NEB, Ipswich, Mass., USA) was used with size-selected, adaptor-ligated cDNA at 37° C. for 15 minutes followed by five minutes at 95° C. before PCR. PCR was performed with Phusion High-Fidelity DNA polymerase, Universal PCR primers and Index (X) Primer. The resulting PCR products were purified (AMPure XP system) and library quality was assessed on the Agilent Bioanalyzer 2100 system. Sequencing was carried out on an Illumina NovaSeq® 6000 (NovoGene; Illumina, San Diego, Calif., United States), using paired-end 2×150 by chemistry at a depth of 20 million reads per sample.

Sequencing quality was examined using FastQC¹² (v0.11.2), and adaptors were subsequently trimmed with Trimmomatic¹³ (v0.36) (in paired-end mode, parameters: TruSeq3-PE-2.fa:2:30:7:8:true LEADING:10 TRAILING:10 SLIDINGWINDOW:5:20 MINLEN:36). Trimmed fastq files were subsequently re-examined with FastQC again to ensure efficient adaptor and quality trimming. Reads were aligned to the hg38 genome (obtained through UCSC) with STAR¹⁴ (v2.7.8a) using the default parameters only for quality control purposes. Aligned reads were subjected to the following quality control metrics: (i) duplication rate assessment through Picard Markduplicates (v.2.18), (ii) rRNA content, genomic read distribution and 3′-5′ bias through RNA-SeQC (v.2.4.0). These metrics, together with the read alignment summary from STAR, were summarized using MultiQC¹⁵ (v1.9), resulting in the removal of one sample on the basis of abnormal read duplication rates (n=1 mild disease). Gene quantification was performed using Salmon¹⁶ (v1.3.0) with adaptor-trimmed reads and gene annotation obtained from GENCODE version 31 (parameters: —gcBias-validateMappings). Gene-level read counts were imported into R using R package Tximport¹³ (v.1.18.0) with transcript length adjustment. Read counts were first normalized by DESeq2¹⁷ (v.1.30.0), with the svaseq( ) function from R package sva (v.3.38.0)¹⁸ being used to subsequently estimate hidden batch effects with n.sv=4. Sva-normalized read counts were obtained by regressing out covariates using the following function: https://github.com/LieberInstitute/jaffelab/blob/master/R/cleaningY.R and were subsequently used for PCA analysis and sample correlation analysis. R package fgsea (v 1.16.0)¹⁹ was used to perform Gene Set Enrichment Analysis with genes ranked based on fold change between the compared conditions. Gene set files were obtained from the Molecular Signatures Database (MSigDB, v7.0) and only C2: curated gene sets and C5: ontology gene sets were used. Pathway enrichment using significantly differentially expressed genes were performed with R package gProfilerR (v.0.7.0)²°. R scripts and essential data files to reproduce the mRNA-seq analysis are available (raw data not presented herein).

HTG EdgeSeq MicroRNA (miRNA) Whole Transcriptome Assay (WTA) from Plasma: Lysis of t₀₋₁ plasma aliquots was facilitated by combining 30 μL plasma with equivalent (v/v) amounts of HTG Plasma Lysis Buffer (HTG Molecular, Tucson, Ariz., USA) as well as 1/10^{th} (v/v) amounts of Proteinase K (HTG Molecular, Tucson, Ariz., USA). The mixture was subsequently incubated for three hours at 50° C. shaking at 1,400 rpm. From each prepared sample, 35 μL were added per well to a 96-well sample plate. Human fetal brain RNA was added to one well at 25 ng/well to serve as an internal control. Samples were run on an HTG EdgeSeq Processor using the HTG EdgeSeq miRNA WTA (HTG Molecular, Tucson, Ariz., USA) to facilitate nuclease protection, whereby a pre-selected miRNA population is protected with proprietary protection probes, followed by degradation of all non-hybridized probes and non-targeted RNA by 51 nuclease. Following the processing, samples were individually barcoded (using a 16-cycle PCR reaction), individually purified using AMPure XP beads (Beckman Coulter, Brea, Calif., USA), and quantified using a KAPA Library Quantification kit (KAPA Biosystem, Wilmington, Mass., USA). The library was sequenced on a NextSeq (Illumina, Inc., San Diego, Calif.) using a V3 150-cycle kit with two index reads. PhiX (Roche, Mississauga, ON, CAN) was spiked into the library at 5%; this spike-in control is standard for Illumina sequencing libraries. Data were returned from the sequencer in the form of demultiplexed FASTQ files, with one file per original well of the assay. The HTG EdgeSeq Parser (v. 5.0.535.3181, HTG Molecular, Tucson, Ariz., USA) was used to align the FASTQ files to the probe list to collate the data. Data were provided as data tables of raw, quality control (QC) raw, counts per million, and median normalized.

HTG EdgeSeq MiRNA Analysis: Samples were initially analyzed using three QC metrics: (i) QC0, examining degraded sample; cut-off of positive % ≥14% as failure, (ii) QC1, insufficient read depth; read depth 500k as failure, (iii) QC2, minimal expression variability; relative standard deviation of reads 0.08 as failure. Of the 156 samples sent for sequencing, 12 exhibited failure at the level of QC2 and were excluded from all downstream analyses. Normalization of miRNA expression data on the remaining samples was performed using DeSeq2¹⁷ (v. 1.14.1) in the HTG reveal software (v.3.0.0, HTG Molecular, Tucson, Ariz., USA). MiRNAs were considered detectable if they had expression levels of >5 counts per million in more than half of the samples. Expression counts were logarithmically scaled (log 10) for data visualization.

Simultaneous Multiplexed In Vitro Immunofluorescence: Cells were fixed in ice-cold methanol (Sigma-Aldrich, St Louis, Mo.) for five minutes at room temperature. Blocking was subsequently conducted in a solution containing 1% BSA (w/v, BioShop, Burlington, ON, CAN), 22.52 mg/mL glycine (ThermoFisher, Waltham, Mass., USA), and PBS^{-/-} with Tween 20 (PBST, PBS^{-/-}+0.1% Tween 20) for 30 minutes. Immunostaining was then conducted with diluted antibody (Table II) in 1% BSA PBST for 16 hours at 4° C. The cells were subsequently washed three times in PBS^{-/-}, five minutes for each wash, and re-incubated with the concordant secondary antibodies in 1% BSA for one hour at room temperature in the dark. Post-washing, cells were counterstained with Vectashield Antifade Mounting Media with 4′,6-diamidino-2-phenylindole ([DAPI], Vector Laboratories, Burlingame, Calif., USA) and mounted with coverslips (VWR International, Mississauga, ON, CAN) and sealed with nail polish. Confocal images were taken using an Olympus Fluoview 1000 Confocal microscope Olympus IX81 inverted stand (Olympus, Calif., USA). Fluorochromes were excited using the following wavelengths: 405 nm for DAPI, 473 nm for Alexa Fluor 488, and 559 nm for Alexa Fluor 568 (ThermoFisher, Waltham, Mass., USA). A 20X/0.75NA UPLSAPO super apochromat objective was used to take the 20X images while a Plan Apo 40x/1.35 NA oil immersion objective was utilized for the 40X images. Image processing was done with the FV10-ASW 4.2 Viewer (Olympus, Calif., USA). Image intensities were calculated using FIJI²¹ (v2.1.0/1.53c), by examining the integrated density and dividing that by the number of DAPI positive cells within each image.

Data Visualization and Statistical Analysis: All data generated and analyzed which support the findings of this study are included in this article. Associated supplementary information files are available on a publicly accessible archive (see below). Descriptive Analysis—Clinical characteristics were characterized using summary statistics. Continuous variables were described using median and inter-quartile range (IQR), and dichotomous or polytomous variables were described using frequencies. Between-group differences were evaluated using Wilcoxon rank-sum tests for continuous variables and Fisher's exact tests for dichotomous/polytomous variables. Correlation between continuous variables were quantified using Spearman rank correlation. Descriptive outcome analysis—The Kaplan-Meier survival method was applied to assess the in-hospital death, and the between-group differences in the freedom from death were evaluated using log-rank tests. The length of hospitalization/ICU was characterized using competing risk models in terms of cumulative incidence rate function. Univariable Cox proportional hazard regression were applied to assess and quantify the association of the baseline clinical characteristics with in-hospital/ICU death. The associations of continuous variables were modeled using natural cubic splines. Biomarker Analysis—Comparisons between two independent groups were made using t-tests for normally distributed continuous variables or Wilcoxon rank-sum tests non-normally distributed continuous variables. When more than two groups were compared, either a one-way ANOVA with a Tukey or Bonferroni post-hoc test (where appropriate) for multiple testing correction, Kruskal-Wallis one-way analysis of variance with Dunn's multiple comparison correction. Two-way ANOVA was used to estimate how the mean quantitative variable changes according to time and group differences in leak experiments. Where appropriate, Benjamini-Hochberg false discovery rate (FDR) was utilized with adjusted P values (or Q value where stated) of <0.05 being considered statistically significant and indicated in the graphs as reported by the analysis software with significance thresholds of P<0.05, P<0.01, P<0.001, and P<0.0001 indicated as *, **, ***, **** respectively. MiRNA pathway analysis was conducted using BioCarta/KEGG/Reactome databases and tested for enrichment by a hypergeometric test with adjustment for multiple comparisons using the Benjamini-Hochberg FDR, with P<1.05 considered to be statistically enriched in a gene set of interest.²²⁻²⁴ Although many hypotheses were tested throughout the manuscript, no experiment-wide multiple test correction was applied. Unless indicated otherwise, graphs depict averaged values of independent data points with technical replicates and have error bars displayed as mean +/−standard deviation (±S.D.). Data were analyzed with GraphPad Prism 9.0.0 for MacOS (GraphPad Software, Inc., La Jolla, Calif., USA; Biomarker Multiple Comparisons), R²⁵ (v4.0.3; Spearman Correlation Plots), and FIJI²¹ (v2.1.0/1.53c; Quantifying Image Intensities). Final figures were assembled for publication purposes using Adobe Illustrator (v25.4.1).

Risk Assessment Using Machine Learning: 250 experiments were performed using repeated randomized stratified sub-sampling cross-validation into 80% training and 20% testing using Python (v3.8.8) and scikit-learn²⁶ (v.0.24.1). Categorical features were hot encoded, with missing variables recorded as additional categorical variables, having −1.0 for numerical features. For each experiment, a Random Decision Forest model was fit to the training dataset and evaluated on the independent testing set.²⁷ Model performance was assessed by the area under the receiver operating characteristic (AUROC) calculated on the testing set. Average AUROC and 95% confidence intervals were calculated across the 250 runs using the percentile method. Feature importance was estimated using permutation feature importance and aggregated across the 250 runs. The microRNA model had a multiphase selection, whereby all microRNA were inputted into the first phase (feature selection via collinearity), upon which the remaining 102 microRNA were used to train the model.

Data Deposit: The data generated in this study have been deposited in the National Center for Biotechnology Information's Gene Expression Omnibus and are accessible through the GEO Series accession number (GSE; GSE178331, mRNA) and (GSE178246, miRNA).

Supplemental Tables

TABLE I
Key Reagents and Resources

| REAGENT or RESOURCE | SOURCE | IDENTIFER |
|---|---|---|
| Enzyme-Linked Immunosorbent Assays | | |
| Ang-2, sE-SEL, SICAM-1, SVCAM-1 | ProteinSimple | Cat#: SPCKC-PS-004112 |
| ET-1, IL-6, IL-8, sTREM-1 | ProteinSimple | Cat#: SPCKC-PS-004111 |
| High Sensitivity Cardiac Troponin I | Biomatik | Cat#: EKU09460 |
| MPO | R & D Systems | Cat#: DMYE00B |
| sSlit2 | Elabscience | Cat#: E-EL-H0931 |
| SARS-CoV-2 Spike (Trimer) Ig Total | ThermoFisher | Cat#: BMS2323 |
| Chemicals, Peptides, and Recombinant Proteins | | |
| Bovine Serum Albumin | BioShop | Cat#: ALBC0100 |
| Dexamethasone | BioShop | Cat#: DEX002.100 |
| EBM-2 ™ | Lonza | Cat#: 00190860 |
| EGM-2 ™ Bullet Kit | Lonza | Cat#: CC-3162 |
| FITC-40 kDa Dextran | Sigma-Aldrich | Cat#: 53379 |
| Gelatin | ThermoFisher | Cat#: S006100 |
| Hanks' Balanced Salt Solution | Thermo | Cat#: 14025092 |
| Methanol | Sigma-Aldrich | Cat#: 322415 |
| Mounting Medium with DAPI | Vectashield | Cat#: H-1200 |
| Nangibotide | LifeTein | Cat#: Custom Order |
| Phosphate Buffered Saline | Gibco | Cat#: LS10010023 |
| RIPA Buffer (10×) | EMD Millipore | Cat#: 20-188 |
| Slit2-N | PreproTech | Cat#: 150-11 |
| Thrombin | Sigma-Aldrich | Cat#: 10602400001 |
| TNFα Human | Sigma-Aldrich | Cat#: T0157-10UG |
| TritonX-100 | Sigma-Aldrich | Cat#: T8787 |
| Tween20 | ThermoFisher | Cat#: 003005 |
| UltraPure Glycine | ThermoFisher | Cat#: 15527013 |
| Commercial Components | | |
| Vacutainer EDTA Tubes | BD Diagnostics | Cat#: 367525 |
| E-16 Plates | Agilent | Cat#: 300601150 |
| Black 96 well assay plate | Sigma-Aldrich | Cat#: MO312 |
| Costar TC-Treated Plates (24-well) | Millipore Sigma | Cat#: CLS3527-100EA |
| Coverslips No. 1, 24 × 50 mm | VWR | Cat#: 4839081 |
| DNA LoBind Tubes | Eppendorf | Cat#: 22431021 |
| Mycoplasma Contamination Kit | ThermoFisher | Cat#: 4460623 |
| RNeasy Plus Micro Kit | Qiagen | Cat#: 74034 |
| Transwell Filters (12-well plate) | Corning | Cat#: 3462 |
| NEBNext ® UltraTM Library Prep Kit | New England Biolabs | Cat#: E7645S |
| AMPure XP Beads | Beckman Coulter | Cat#: A63880 |
| Uracil-Specific Excision Reagent | New England Biolabs | Cat#: M5505S |
| Phusion High-Fidelity DNA | ThermoFisher | Cat#: F-530XL |
| polymerase | | |
| HTG Lysis Buffer | HTG Diagnostic’s | Cat#: SPP-Mi-04 |
| KAPA Library Quantification Kit | Roche | Cat#: 07960140001 |
| PhiX | Illumina | Cat#: FC-110-3001 |
| Cells | | |
| pHUVEC | Lonza | Cat#: C2519ALot: 661173 |
| | | Cat#: C2519ALot: 636514 |
| Key Instruments | | |
| Cytation 5 | Biotek | Serial Number: 16041913 |
| DS-11 + Spectrophotometer | DeNovix | Serial Number: 760419B |
| ELLA | ProteinSimple | Serial Number: ELLA-16080112 |
| NextSeq | Illumina | Serial Number: A0877 |
| NovoSeq6000 | Illumina | Serial Number: |
| Fluoview 1000 Confocal IX81 | Olympus | Serial Number: 8B03839 |
| Real-Time Cell Analysis | xCelligence | Serial Number: 3211107167871 |
| Software and Algorithms | | |
| Deposited Data | | |
| Plasma microRNA transcriptome | Human participants | GSE: 178246 |
| Messenger RNA sequencing | Pooled HUVECs | GSE: 178331 |

Abbreviations:
Ang-2 = Angiopoietin-2; Cat# = Catalogue number; DAPI = 4′,6-diamidino-2-phenylindole; EBM = Endothelial basal media; EGM = Endothelial growth medium; ET-1 = Endothelin-1; FITC = Fluorescein isothiocyanate; pHUVEC = Pooled human umbilical vein endothelial cells; Ig = Immunoglobulin; IL = Interleukin; kDa = Kilodalton; MPO = Myeloperoxidase; RIPA = Radioimmunoprecipitation assay buffer; SARS-CoV-2 = Severe acute respiratory syndrome coronavirus; sICAM = Soluble intercellular adhesion molecule-1; sSLIT-2 = Soluble slit guidance ligand 2; sTREM-1 = Soluble triggering receptor expressed on myeloid cells-1; sVCAM-1 = Soluble vascular cell adhesion molecule-1; TC = Tissue culture; TNFα = Tumor necrosis factor alpha.

TABLE II
Antibodies

| ANTIGEN | HOST | SOURCE | IDENTIFER |
|---|---|---|---|
| Human Vascular Endothelial- | Mouse | R & D Systems | Cat#: 9381; RRID:AB_2260374 |
| Cadherin | | | |
| Polyclonal Claudin-5 | Rabbit | Invitrogen | Cat#: 34-1600; RRID:AB_2533157 |
| Alexa Fluor 488 Anti-rabbit | Goat | ThermoFisher | Cat#: A11008; RRID:AB_143165 |
| Alexa Fluor 568 Anti-mouse | Donkey | ThermoFisher | Cat#: A10042; RRID:AB_2534017 |

Abbreviations: Cat# = Catalogue number; Research Resource Identifier (Antibody) = RRID:AB

TABLE III
Admission Clinical Laboratory Findings

| | Disease Severity | | | | | |
|---|---|---|---|---|---|---|
| | Mild | | | | Severe | |
| | Negative | Mild | Moderate | Severe | Negative | |
| Characteristics* | (n = 30) | (n = 27) | (n = 39) | (n = 76) | (n = 69) | P-value |

Complete Blood Count

| | | | | | | | | | | | |
|---|---|---|---|---|---|---|---|---|---|---|---|
| White blood cells - million/mm³ | | | | | | | | | | | |
| Median (IQR) | 8.0 | (6.1-10.0) | 7.0 | (5.2-11.2) | 7.0 | (5.1-9.1) | 10.8 | (8.3-13.8) | 11.9 | (7.4-16.9) | <0.001 |
| Distribution - no./total no. (%) | | | | | | | | | | | |
| <4 | 3/30 | (10.0) | 4/27 | (14.8) | 4/36 | (11.1) | 2/75 | (2.7) | 4/69 | (5.8) | 0.1954 |
| >11 | 3/30 | (10.0) | 7/27 | (25.9) | 6/36 | (16.7) | 35/75 | (46.7) | 36/69 | (52.2) | <0.0001 |
| Neutrophils - per mm³ | | | | | | | | | | | |
| Median (IQR) | 5.1 | (3.8-7.0) | 4.7 | (3.6-8.2) | 4.7 | (3.2-6.5) | 9.0 | (6.0-12.0) | 9.5 | (6.0-15.0) | <0.001 |
| <3,000 | 4/30 | (13.3) | 5/27 | (18.5) | 8/36 | (22.2) | 1/75 | (1.3) | 3/68 | (4.4) | 0.0009 |
| >5,800 | 11/30 | (36.7) | 10/27 | (37.0) | 12/36 | (33.3) | 22/75 | (29.3) | 14/68 | (20.6) | 0.3613 |
| Lymphocytes - per mm³ | | | | | | | | | | | |
| Median (IQR) | 1.4 | (1.0-1.9) | 1.3 | (0.9-2.1) | 1.2 | (0.8-1.7) | 0.9 | (0.6-1.2) | 0.9 | (0.6-1.5) | 0.0023 |
| Distribution - no./total no. (%) | | | | | | | | | | | |
| <1,500 | 16/30 | (53.3) | 17/27 | (63.0) | 25/36 | (69.4) | 61/75 | (81.3) | 52/68 | (76.5) | 0.0343 |
| >3,000 | 1/30 | (3.3) | 1/27 | (3.7) | 2/36 | (5.6) | 3/75 | (4.0) | 4/68 | (5.9) | 0.9693 |
| Monocytes - per mm³ | | | | | | | | | | | |
| Median (IQR) | 0.6 | (0.4-0.8) | 0.6 | (0.4-0.8) | 0.6 | (0.3-0.7) | 0.5 | (0.3-0.8) | 0.8 | (0.3-1.0) | 0.5077 |
| Distribution - no./total no. (%) | | | | | | | | | | | |
| <300 | 1/30 | (3.3) | 4/27 | (14.8) | 5/36 | (13.9) | 14/75 | (18.7) | 21/68 | (30.9) | 0.0180 |
| >500 | 16/30 | (53.3) | 15/27 | (55.6) | 18/36 | (50.0) | 32/75 | (42.7) | 42/68 | (61.8) | 0.2459 |
| Platelet count - thousand/mm³ | | | | | | | | | | | |
| Median (IQR) | 232 | (179-292) | 247 | (177-273) | 246 | (179-292) | 227 | (154-313) | 217 | (151-268) | 0.6530 |
| Distribution - no./total no. (%) | | | | | | | | | | | |
| <150,000 | 2/30 | (6.7) | 4/27 | (14.8) | 3/36 | (8.3) | 17/75 | (22.7) | 17/69 | (24.6) | 0.1117 |
| >400,000 | 2/30 | (6.7) | 1/27 | (3.7) | 5/36 | (13.9) | 6/75 | (8.0) | 5/69 | (7.2) | 0.6457 |

Cardiac Laboratory Results

| | | | | | | | | | | | |
|---|---|---|---|---|---|---|---|---|---|---|---|
| hs-CnT - ng/mL, median (IQR) | 2.50 | (0.92-6.20) | 0.92 | (0.92-6.50) | 1.90 | (0.92-7.66) | 16.4 | (7.42-56.0) | 23.0 | (10.0-62.5) | <0.0001 |

Coagulation Laboratory Results

| | | | | | | | | | | | |
|---|---|---|---|---|---|---|---|---|---|---|---|
| International normalized ratio | | | | | | | | | | | |
| <0.9 | 0/11 | (0.0) | 0/5 | (0.0) | 0/17 | (0.0) | 0/74 | (0.0) | 0/65 | (0.0) | 1.0000 |
| >1.1 | 5/11 | (45.5) | 4/5 | (80.0) | 12/17 | (70.6) | 47/74 | (63.5) | 45/65 | (69.2) | 0.5329 |
| Partial thromboplastin time | | | | | | | | | | | |
| <25 | 6/8 | (75.0) | 2/5 | (40.0) | 2/14 | (14.3) | 0/74 | (0.0) | 0/64 | (0.0) | <0.0001 |
| >40 | 0/8 | (0.0) | 1/5 | (20.0) | 3/14 | (21.4) | 10/74 | (13.5) | 4/64 | (6.3) | 0.2930 |

Supplemental Laboratory Results

| | | | | | | | | | | | |
|---|---|---|---|---|---|---|---|---|---|---|---|
| Albumin, g/liter | | | | | | | | | | | |
| <35 | 2/5 | (40.0) | 4/9 | (44.4) | 8/17 | (47.1) | 59/74 | (79.7) | 44/69 | (63.8) | 0.0136 |
| >50 | 0/5 | (0.0) | 0/9 | (0.0) | 0/17 | (0.0) | 1/74 | (1.4) | 0/69 | (0.0) | 0.8513 |
| Alanine aminotransferase, | 1/14 | (7.1) | 5/18 | (27.8) | 3/24 | (12.5) | 25/72 | (34.7) | 17/66 | (25.8) | 0.0791 |
| >40 U/liter | | | | | | | | | | | |
| Aspartate aminotransferase, | 3/14 | (21.4) | 5/18 | (27.8) | 4/24 | (16.7) | 41/72 | (56.9) | 26/66 | (39.4) | 0.0019 |
| >40 U/liter | | | | | | | | | | | |
| Creatine kinase, ≥200 U/liter | 2/13 | (15.4) | 2/5 | (40.0) | 3/36 | (8.3) | 54/74 | (73.0) | 35/63 | (55.6) | <0.0001 |
| Creatinine, ≥133 μmol/liter | 1/30 | (3.3) | 4/27 | (14.8) | 6/36 | (16.7) | 23/75 | (30.7) | 20/69 | (29.0) | 0.0149 |
| D-dimer, ≥0.5 mg/liter | 3/8 | (37.5) | 9/10 | (90.0) | 10/12 | (83.3) | 26/49 | (53.1) | 8/56 | (14.3) | <0.0001 |
| Lactate Dehydrogenase - | | | | | | | | | | | |
| μkatals/liter | | | | | | | | | | | |
| <2.34 | 4/8 | (50.0) | 12/15 | (80.0) | 8/12 | (66.7) | 4/38 | (10.5) | 1/60 | (1.7) | <0.0001 |
| >4.68 | 0/8 | (0.0) | 0/15 | (0.0) | 1/12 | (8.3) | 5/38 | (13.2) | 11/60 | (18.3) | 0.2629 |
| Total bilirubin, >17.1 μmol/liter | 2/13 | (15.4) | 1/16 | (6.3) | 0/17 | (0.0) | 17/71 | (23.9) | 12/68 | (17.6) | 0.1047 |
| Minerals, median (IQR) - | | | | | | | | | | | |
| mmol/liter | | | | | | | | | | | |
| Potassium | 3.0 | (3.8-4.4) | 3.9 | (3.7-4.3) | 4.1 | (3.7-4.4) | 4.2 | (3.8-4.3) | 4.1 | (3.7-4.8) | 0.4223 |
| Sodium | 139 | (137-140) | 137 | (136-141) | 138 | (135-140) | 138 | (135-143) | 137 | (135-140) | 0.5582 |

*Summary statistics, where n-value is not provided, are based on the full population indicated in the column heading.
Bolded log-ranked P values are significant (P values < 0.05). Abbreviations: hs-cTnI = High sensitivity cardiac troponin I; IQR = Interquartilerange. Percentages may not add up to 100% due to rounding.

TABLE IV
Association of Baseline Clinical Characteristics to Mortality
Amongst All Admitted COVID-19 Patients.

| | Unadjusted |
|---|---|
| | Log-Rank |
| Variables (Baseline) | P Value |
| Comorbidity-Gout | 0.003 |
| MiRNA-hsa-miR-6080 | 0.009 |
| Comorbidity-Coronary artery disease | 0.010 |
| MiRNA-Ang-2 | 0.015 |
| MiRNA-hsa-miR-199a-3p | 0.017 |
| MiRNA-hsa-miR-4793-5p | 0.027 |
| MiRNA-hsa-miR-181a-5p | 0.028 |
| MiRNA-hsa-miR-mir-30b-5p | 0.035 |
| MiRNA-hsa-miR-6750-5p | 0.036 |
| Clinical-Age of patient at hospital admission | 0.046 |
| Protein-MPO | 0.079 |
| History of stroke | 0.115 |
| Comorbidity-Heart failure | 0.149 |
| Comorbidity-Obesity | 0.151 |
| Protein-IL-8 | 0.156 |
| Comorbidity-CKD | 0.164 |
| MiRNA-hsa-miR-301a-3p | 0.167 |
| Protein-sVCAM-1 | 0.200 |
| MiRNA-hsa-miR-mir-30c-5p | 0.200 |
| CV Medication-Anticoagulant | 0.230 |
| Clinical-Male | 0.240 |
| History of dyslipidemia | 0.250 |
| Comorbidity-OSA | 0.250 |
| MiRNA-hsa-miR-146a-5p | 0.260 |
| MiRNA-hsa-miR-miR-30e-5p | 0.260 |
| Protein-IL-6 | 0.280 |
| MiRNA-hsa-miR-1 | 0.280 |
| Comorbidity-Malignancy | 0.280 |
| History of cardiac procedure/surgery | 0.300 |
| Comorbidity-GERD | 0.310 |
| History of any prior CV procedure | 0.310 |
| History of arrhythmia | 0.320 |
| Protein-sTREM-1 | 0.340 |
| Protein-E-Selectin | 0.350 |
| Comorbidity-Renal disease | 0.360 |
| MiRNA-hsa-miR-mir-339-3p | 0.380 |
| Comorbidity-Immunocompromised | 0.390 |
| Protein-sICAM-1 | 0.400 |
| CV medication-Number of medications | 0.430 |
| CV medication-ARB | 0.430 |
| MiRNA-hsa-miR-mir-4706 | 0.440 |
| Other CV condition-PFO | 0.440 |
| Comorbidity-Hypertension | 0.480 |
| History of myocardial infarction | 0.490 |
| Other CV condition-Aortic aneurysm | 0.490 |
| CV medication-Statin | 0.510 |
| Comorbidity-Diabetes | 0.520 |
| CV medication-ACE inhibitor | 0.520 |
| CV medication-CCB | 0.530 |
| Other CV condition-TIA | 0.550 |
| Other CV condition-Ischemic heart disease | 0.560 |
| Comorbidity-Vascular disease | 0.620 |
| Comorbidity-Pneumonia | 0.660 |
| Other CV condition-PAD | 0.680 |
| Comorbidity-COPD | 0.690 |
| MiRNA-hsa-miR-mir-26a-5p | 0.720 |
| Clinical-Patient ethnicity | 0.730 |
| CV medication-Beta blocker | 0.740 |
| MiRNA-hsa-miR-mir-30d-5p | 0.750 |
| CV medication-Diuretics | 0.760 |
| Comorbidity-CF | 0.800 |
| Other CV condition-Endocarditis | 0.800 |
| Other CV condition-Dilatated aortic root | 0.800 |
| Other CV condition-Becker’s muscle dystrophy | 0.800 |
| Comorbidity-Asthma | 0.850 |
| Comorbidity-Valvular heart disease | 0.930 |
| Other CV condition-VTE | 0.950 |
| Other CV condition-NSTEMI | 1.000 |

*Gout (n = 3) was a small number of observations.
Bolded log-ranked P values are significant (P values < 0.05).
Abbreviations: ACE = Angiotensin-converting-enzyme inhibitors; Ang-2 = Angiopoetin-2; ARB = Angiotensin II receptor blockers; CCB = Calcium channel blocker; CKD = Chronic Kidney Disease; CF = Cystic Fibrosis; COPD = Chronic obstructive pulmonary disease; GERD = Gastroesophageal reflux disease; IL = Interleukin; miR = MicroRNA; MPO = Myeloperoxidase; NSTEMI = Non-ST-elevation myocardial infarction; OSA = Obstructive Sleep Apnea; PAD = Peripheral arterial disease; PFO = Patent foramen ovale; sICAM, Soluble intercellular adhesion molecule-1; sTREM-1, Soluble triggering receptor expressed on myeloid cells-1; sVCAM-1 = Soluble vascular cell adhesion molecule-1; TIA = Transient ischemic attack; VTE = Venous thromboembolism.

TABLE V
Intensive Care Unit-Level Clinical Characteristics and Clinical Outcomes

| | Severe | Severe Negative | |
|---|---|---|---|
| Characteristics* | (n = 76) | (n = 69) | P-value |
| Demographics | | | |
| Age, median (IQR)-yr. | 61 (52-71) | 61 (51-72) | 0.5137 |
| Distribution-no. (%) | | | |
| 18-40 yr. | 2 (2.6) | 11 (15.9) | 0.0071 |
| 41-64 yr. | 45 (59.2) | 26 (37.7) | 0.0126 |
| ≥65 yr. | 29 (38.2) | 32 (46.4) | 0.3999 |
| Male Sex, no./total no. (%) | 52/76 (68.4) | 46/69 (66.7) | 0.8602 |
| BMI, median (IQR)^{†} | 28.1 (24.2-31.8) | 25.7( 21.8-32.5) | 0.3641 |
| Obesity^{‡}-no. (%) | 38/61 (62.3) | 35/69 (50.7) | 0.2168 |
| Length of hospital stay, median | 13 (7-35) | 8 (3-15) | <0.0001 |
| (IQR)-days† | | | |
| Max temperature, median (IQR), ° C. | 36.8 (36.3-37.8) | 36.4 (35.8-37.1) | 0.0797 |
| Mix temperature, median (IQR), ° C. | 36.7 (36.3-37.3) | 36.3 (35.6-37.1) | 0.0815 |
| Illness Severity | | | |
| APACHE | 21 (16-27) | 20 (16-29) | 0.8521 |
| SOFA score | | | |
| Median, (IQR) | 9 (4-10) | 7 (3-10) | 0.8112 |
| ≥2-no. (%) | 12/38 (31.6) | 18/69 (26.1) | 0.3674 |
| ≥6-no. (%) | 24/38 (63.2) | 44/69 (63.8) | >0.9999 |
| Respiratory metrics | | | |
| Proned-no. (%) | 8/38 (21.1) | 3/69 (4.3) | 0.0158 |
| Intubation-no. (%) | 53/76 (69.7) | 47/69 (68.1) | 0.8565 |
| NIV-mean, days† | 3.12 | 1.5 | 0.2177 |
| MIV-mean, days† | 10.84 | 7.61 | 0.2215 |
| EC MO-no. (%) | 21/76 (27.6) | 0 (0.0) | <0.0001 |
| FiO2, median % (IQR) | 0.53 (0.50-0.63) | 0.40 (0.30-0.50) | <0.0001 |
| PF ratio, median (IQR), mm Hg/% | 130 (103-177) | 188 (146-289) | 0.0003 |
| PF ratio <300 mm Hg/% | 26/26(100.0) | 38/49 (77.6) | 0.0126 |
| RR, no. (%), ≥22 breaths/min | 22/38 (57.9) | 33/65 (50.8) | 0.5425 |
| Cardiovascular metrics | | | |
| Heart rate, median (IQR) | 80 (67-101) | 82 (67-97) | 0.6911 |
| Blood Pressure, median (IQR), | | | |
| mmHg | | | |
| Max Systolic | 165 (141-179) | 140 (125-165) | 0.0242 |
| Max Diastolic | 75 (65-84) | 69 (63-80) | 0.1707 |
| Therapies-no. (%) | | | |
| Intravenous antibiotics | 11/38 (28.9) | 20/69 (29.0) | >0.9999 |
| Systemic glucocorticoids | 13/38 (34.2) | 37/67 (55.2) | 0.2016 |
| Remdesivir | 1/38 (2.6) | 0/69 (0.0) | 0.3679 |
| Fludrocortisone | 0/38 (0.0) | 5/69 (7.2) | 0.1583 |
| Outcomes-no. (%) | | | |
| Any secondary CV event | 13/76(17.1) | 1/69 (1.4) | 0.0013 |
| ARDS during hospitalization | 43/76 (56.6) | 25/69 (36.2) | 0.0195 |
| Arrythmia during hospitalization | 8/76 (10.5) | 0/69 (0.0) | 0.0068 |

*Summary statistics, where n-value is not provided, are based on the full population indicated in the column heading
^{†}Body mass index is the weight in kilograms divided by the square of the height in meters.
^{‡}Obesity is classified according to World Health Organization guidelines (i.e., >25).
Bolded log-ranked P values are significant (P values < 0.05).
Abbreviations: APACHE = Acute physiologic assessment and chronic health evaluation; ARDS = Acute respiratory distress syndrome; BMI = Body mass index; CV = Cardiovascular; ECMO = Extracorporeal membrane oxygenation; FiO₂ = Fraction of inspired oxygen; IQR = Interquartile range; MIV = Mechanical ventilation; NIV = Non-invasive ventilation; PF = Ratio of arterial oxygen partial pressure to fractional inspired oxygen; RR = Respiratory rate; S.D. = Standard deviation; SOFA = Sequential Organ Failure Assessment.
Percentages may not add up to 100% due to rounding.

TABLE VI
Association of Baseline Clinical Characteristics to
Mortality Amongst All Severe COVID-19 Patients.

| | Unadjusted |
|---|---|
| | Log-Rank |
| Variables (Baseline) | P Value |
| Comorbidity-Gout | <0.001 |
| MiRNA--miR-30b-5p | 0.005 |
| MiRNA-hsa-miR-199a-3p | 0.006 |
| MiRNA-hsa-miR-181a-5p | 0.010 |
| Protein-VCAM-1 | 0.012 |
| MiRNA-hsa-miR-339-3p | 0.017 |
| MiRNA-hsa-miR-mir-30e-5p | 0.017 |
| Protein-Ang-2 | 0.020 |
| MiRNA-hsa-miR-146a-5p | 0.030 |
| MiRNA-hsa-miR-30c-5p | 0.035 |
| Comorbidity-CKD | 0.038 |
| History of coronary artery disease at | 0.041 |
| baseline | |
| MiRNA-hsa-miR-6080 | 0.042 |
| MiRNA-hsa-miR-6750-5p | 0.051 |
| MiRNA-hsa-miR-4793-5p | 0.060 |
| Protein-ICAM-1 | 0.096 |
| MiRNA-hsa-miR-301a-3p | 0.103 |
| CV medication-Anticoagulant | 0.103 |
| Clinical-Age of patient at hospital | 0.122 |
| admission | |
| Comorbidit-y-OSA | 0.126 |
| Clinical-Male | 0.130 |
| MiRNA-hsa-miR-4706 | 0.131 |
| Comorbidity-Stroke | 0.184 |
| Protein-IL-8 | 0.220 |
| Comorbidity-Heart failure | 0.220 |
| MiRNA-hsa-miR-26a-5p | 0.230 |
| Protein-sTREM-1 | 0.230 |
| History of arrhythmia | 0.240 |
| Comorbidity-Obesity | 0.260 |
| Comorbidity-COPD | 0.260 |
| MiRNA-hsa-miR-1 | 0.270 |
| History of any prior CV procedure | 0.310 |
| Protein-IL-6 | 0.320 |
| Protein-MPO | 0.320 |
| Comorbidity-GERD | 0.320 |
| Comorbidity-Dyslipidemia | 0.380 |
| Other CV condition-TIA | 0.380 |
| Comorbidity-Peripheral vascular | 0.410 |
| disease | |
| Comorbidity-Immunocompromised | 0.410 |
| Comorbidity-Valvular heart disease | 0.420 |
| Other CV condition-PFO | 0.440 |
| MiRNA-hsa-miR-30d-5p | 0.460 |
| CV medication-Statin | 0.480 |
| CV medication-Diuretics | 0.540 |
| CV medication-Number of | 0.550 |
| medications | |
| CV medication-ARB | 0.550 |
| Comorbidity-Renal disease | 0.560 |
| Other CV condition-Ischemic heart | 0.560 |
| disease | 0.560 |
| Comorbidity-Hypertension | 0.590 |
| CV medication-CCB | 0.590 |
| History of myocardial infarction | 0.600 |
| Comorbidity-Malignancy | 0.600 |
| Other CV condition-Aortic aneurysm | 0.620 |
| CV medication-ACE inhibitor | 0.650 |
| Comorbidity-Pneumonia | 0.660 |
| Clinical-Patient ethnicity | 0.730 |
| Comorbidity-Diabetes | 0.780 |
| Protein-sE-Selectin | 0.810 |
| Comorbidity-Asthma | 0.810 |
| Other CV condition-VTE | 0.840 |
| CV medication-Beta blocker | 0.880 |

*Gout (n = 3) was a small number of observations.
Bolded log-ranked P values are significant (P values < 0.05).
Abbreviations: ACE = Angiotensin-converting-enzyme inhibitors; Ang-2 = Angiopoetin-2; ARB = Angiotensin 11 receptor blockers; CCB = Calcium channel blocker; CKD = Chronic Kidney Disease; CF = Cystic Fibrosis; COPD = Chronic obstructive pulmonary disease; GERD = Gastroesophageal reflux disease; IL = Interleukin; miR = MicroRNA; MPO = Myeloperoxidase; OSA = Obstructive Sleep Apnea; PFO = Patent foramen ovale; sICAM, Soluble intercellular adhesion molecule-1; sTREM-1, Soluble triggering receptor expressed on myeloid cells-1; sVCAM-1 = Soluble vascular cell adhesion molecule-1; TIA = Transient ischemic attack; VTE = Venous thromboembolism.

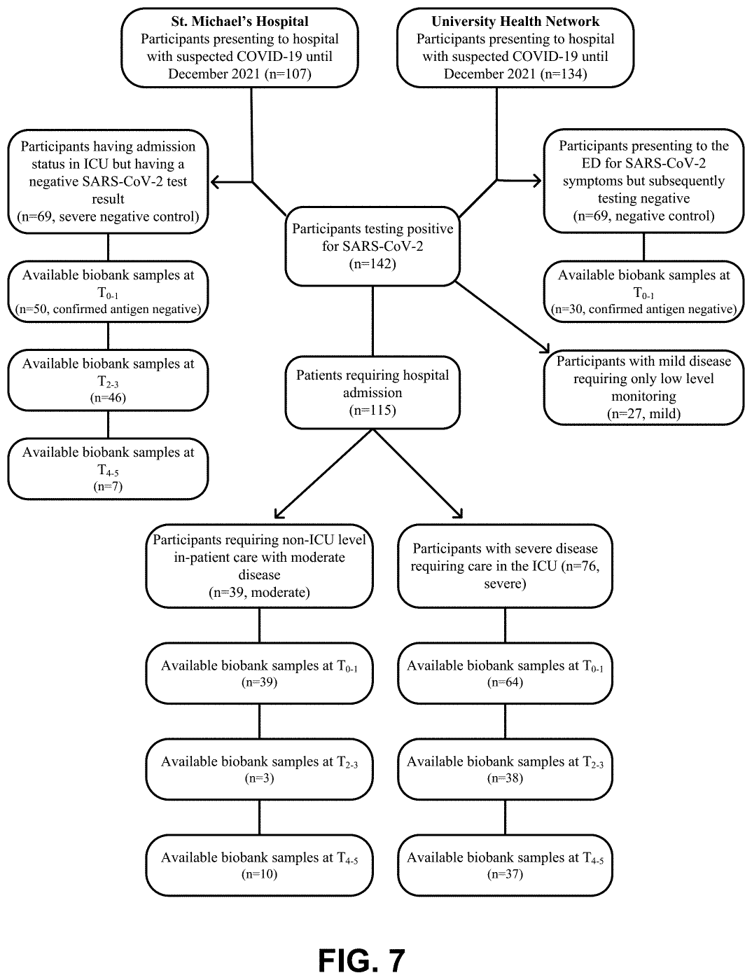

FIG. 7; Related to Methods and Table 1: Flow diagram of patients enrolled between the COLOBILI Study (St. Michael's Hospital) and the COVID Study (University Health Network). Abbreviations: COVID-19=Coronavirus disease 2019; ED=Emergency department; ICU=Intensive care unit.

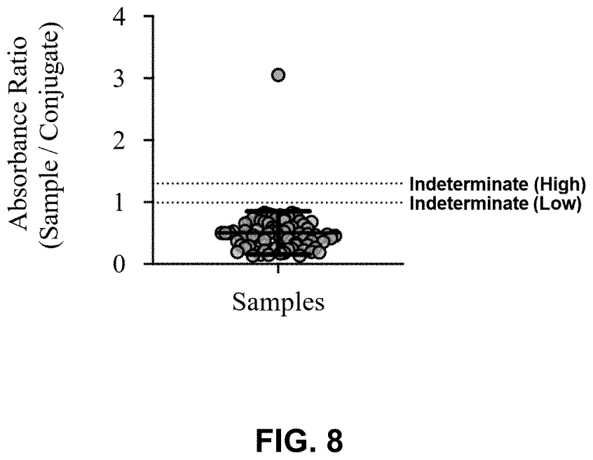

FIG. 8; Related to Methods and Table 1: Spike (trimer) antigen serology testing from patients having a negative SARS-CoV-2 polymerase chain reaction result. The data were analyzed 709 using built-in low and high thresholds, whereby the area between the indeterminate-low and 710 indeterminate-high constitutes an ambiguous result; n=80. Positive control is indicated on the 711 graph (red circle). The graph depicts averaged values of independent technical duplicates data with 712 center bars representing the mean and error bars representing standard deviation (±S.D.).

FIG. 9; Related to FIG. 1: The association of coronary artery disease with mortality characterized in terms of proportion of deceased patients stratified by status. The data were analyzed using log-rank testing. Abbreviations: CAD=Coronary artery disease.

FIG. 10; Related to FIG. 2: Spearman correlations between t₀₋₁ concentrations of biomarkers amongst the entire cohort (SARS-CoV-2 negative and positive populations). Abbreviations: Ang-2=Angiopoietin-2; ET-1=Endothelin-1; Hs-CTnI=High-sensitivity cardiac troponin I; IL=Interleukin; MPO=Myeloperoxidase; sICAM=Soluble intercellular adhesion molecule-1; sTREM-1=Soluble triggering receptor expressed on myeloid cells-1; sVCAM-1=Soluble vascular cell adhesion molecule-1; WBC=White blood cells.

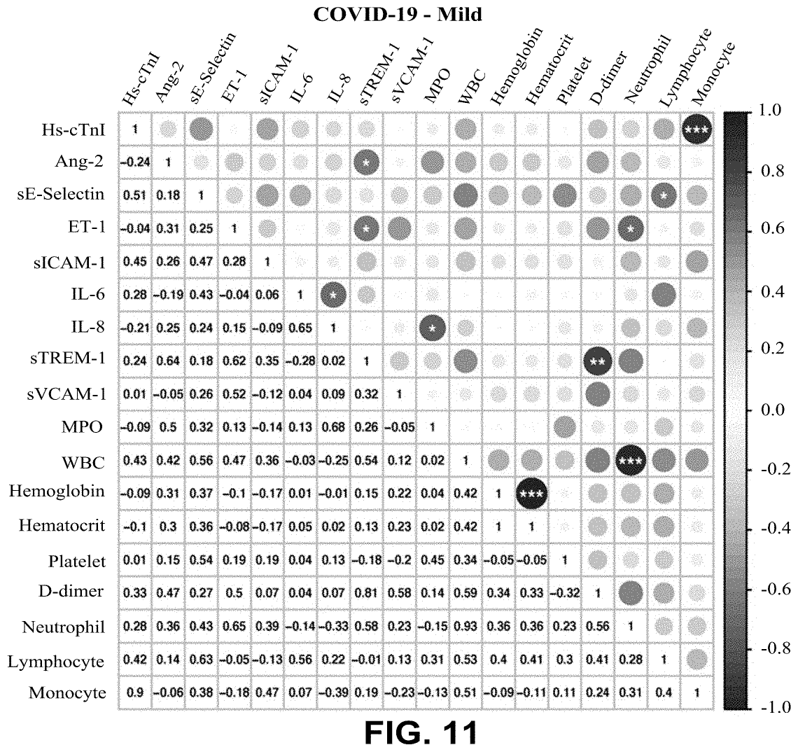

FIG. 11; Related to FIG. 2: Spearman correlations between t₀₋₁ concentrations of biomarkers within the mild COVID-19 subgroup. Abbreviations: Ang-2=Angiopoietin-2; ET-1=Endothelin-1; Hs-cTnI=High-sensitivity cardiac troponin I; IL=Interleukin; MPO=Myeloperoxidase; sICAM=Soluble intercellular adhesion molecule-1; sTREM-1=Soluble triggering receptor expressed on myeloid cells-1; sVCAM-1=Soluble vascular cell adhesion molecule-1; WBC=White blood cells.

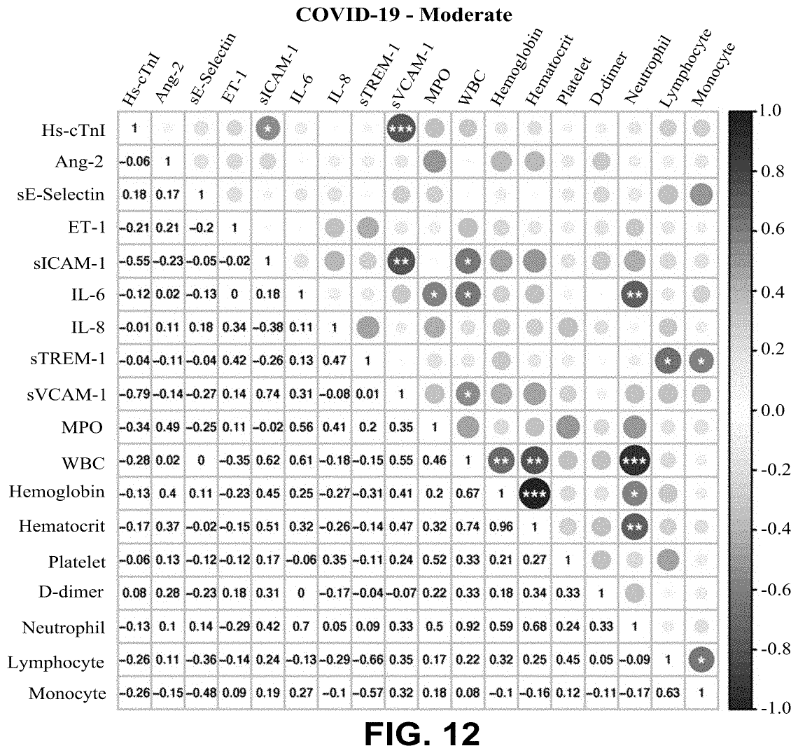

FIG. 12; Related to FIG. 2: Spearman correlations between t₀₋₁ concentrations of biomarkers within the severe COVID-19 subgroup. Abbreviations: Ang-2=Angiopoietin-2; ET-1 =Endothelin-1; Hs-cTnI=High-sensitivity cardiac troponin I; IL=Interleukin; MPO=Myeloperoxidase; sICAM=Soluble intercellular adhesion molecule-1; sTREM-1=Soluble triggering receptor expressed on myeloid cells-1; sVCAM-1=Soluble vascular cell adhesion molecule-1; WBC=White blood cells.

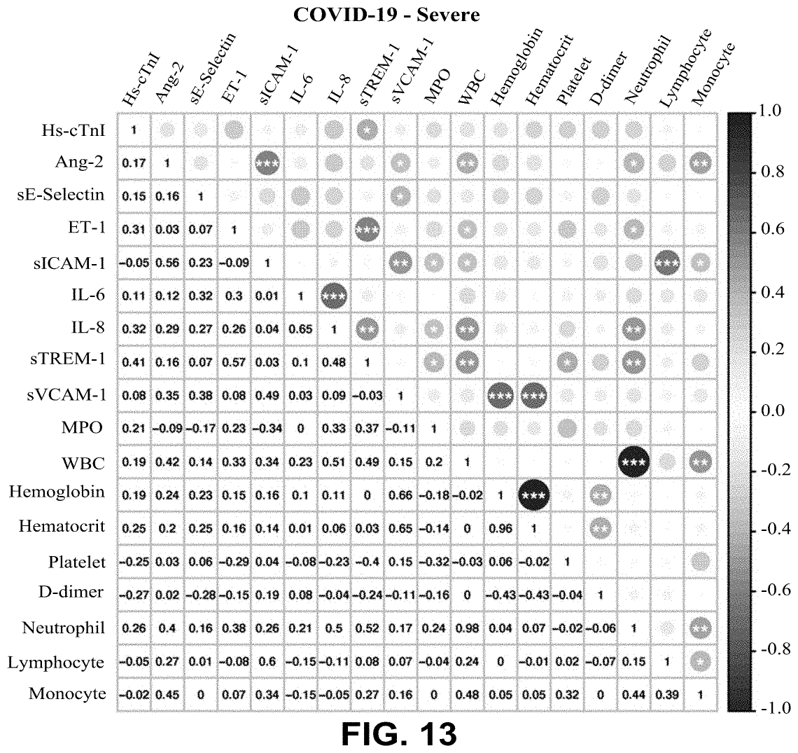

FIG. 13; Related to FIG. 2: Spearman correlations between t₀₋₁ concentrations of biomarkers within the COVID-19 subgroup. Abbreviations: Ang-2=Angiopoietin-2; ET-1=Endothelin-1; Hs-cTnI=High-sensitivity cardiac troponin I; IL=Interleukin; MPO=Myeloperoxidase; sICAM=Soluble intercellular adhesion molecule-1; sTREM-1=Soluble triggering receptor expressed on myeloid cells-1; sVCAM-1=Soluble vascular cell adhesion molecule-1; WBC=White blood cells.

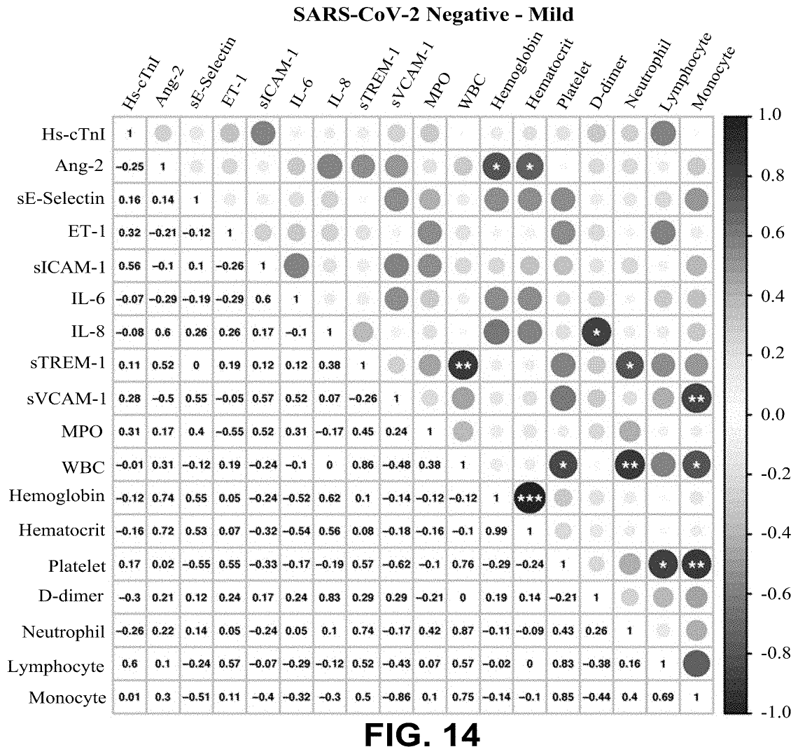

FIG. 14; Related to FIG. 2: Spearman correlations between t₀₋₁ concentrations of biomarkers within the mild SARS-CoV-2 negative subgroup. Abbreviations: Ang-2=Angiopoietin-2; ET-1=Endothelin-1; Hs-cTnI=High-sensitivity cardiac troponin I; IL=Interleukin; MPO=Myeloperoxidase; sICAM=Soluble intercellular adhesion molecule-1; sTREM-1=Soluble triggering receptor expressed on myeloid cells-1; sVCAM-1=Soluble vascular cell adhesion molecule-1; WBC=White blood cells.

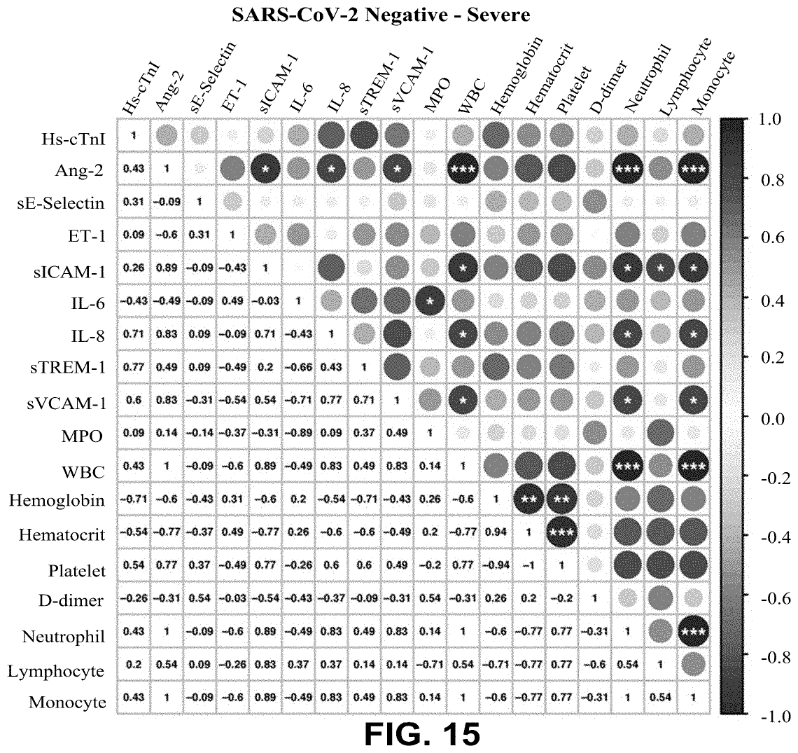

FIG. 15; Related to FIG. 2: Spearman correlations between t₀₋₁ concentrations of biomarkers within the severe SARS-CoV-2 negative subgroup. Abbreviations: Ang-2=Angiopoietin-2; ET-1=Endothelin-1; Hs-cTnI=High-sensitivity cardiac troponin I; IL=Interleukin; MPO=Myeloperoxidase; sICAM=Soluble intercellular adhesion molecule-1; sTREM-1=Soluble triggering receptor expressed on myeloid cells-1; sVCAM-1=Soluble vascular cell adhesion molecule-1; WBC=White blood cells.

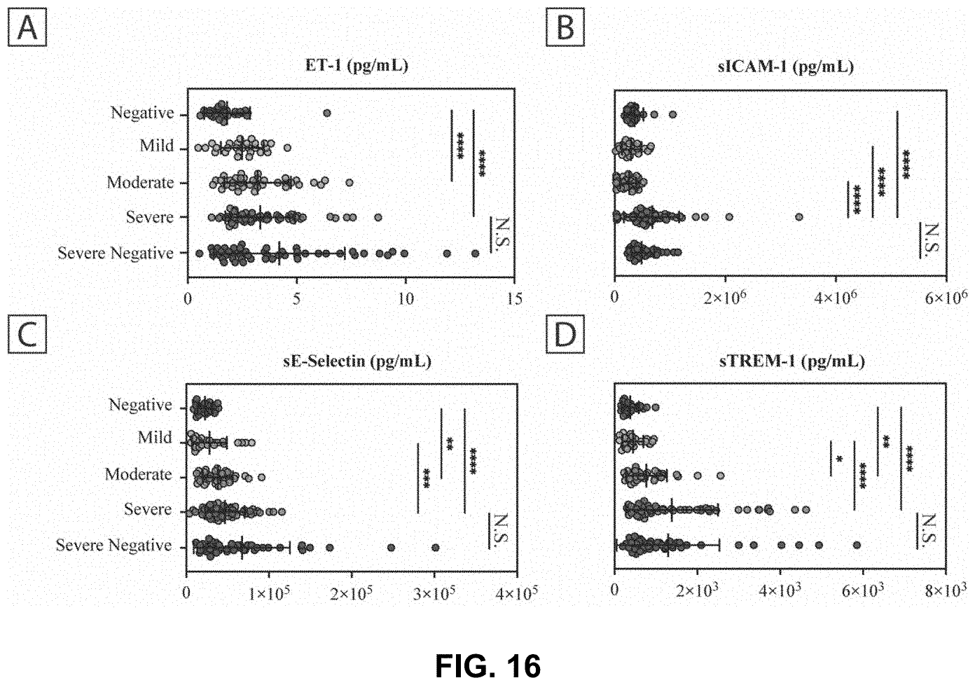

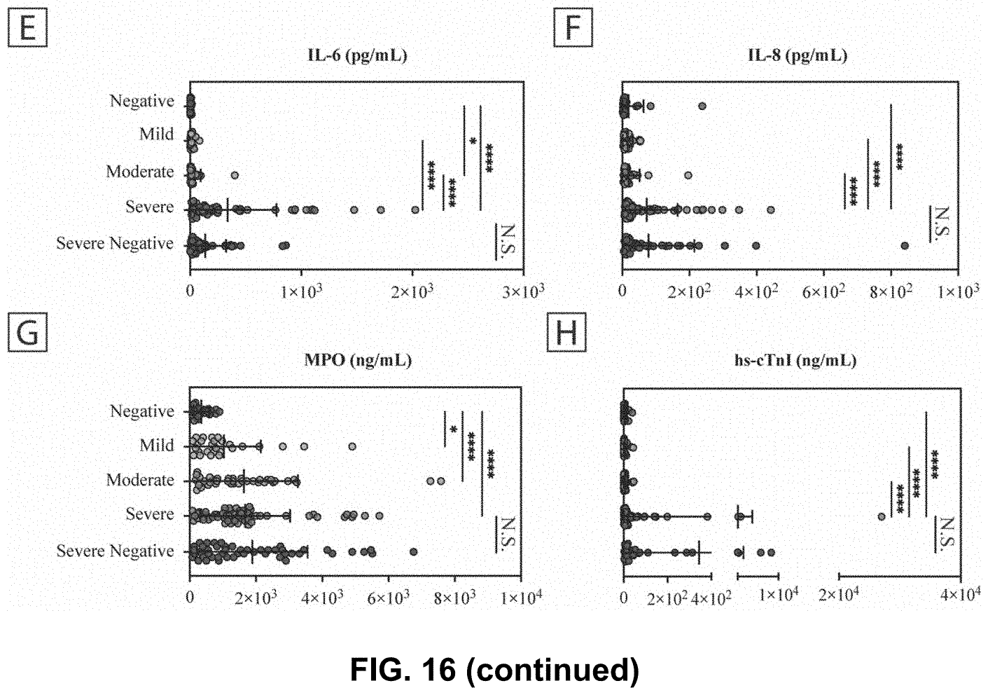

FIG. 16; Related to FIG. 2. Plasma Concentration of Endothelial Dysfunction and Inflammatory Markers at t₀₋₁. (a) ET-1, (b) sICAM-1, (c) sE-Selectin, (d) sTREM-1, (e) IL-6, (f) IL-8, (g) MPO, and (h) hs-cTnI stratified among disease severity. Data shown are for all patients with an available t₀₋₁ sample (n=210), with values representing the mean and error bars are (±S.D.). P values for multiple group comparisons were determined by Kruskal-Wallis test with Dunn's multiple comparisons test. Severe negative comparisons are only shown in reference to the concordant severe group; testing was conducted with all groups. The graph depicts averaged values of independent technical triplicates data points with center bars representing the mean and error bars representing standard deviation (±S.D.). Abbreviations: ET-1=Endothelin-1; hs-cTnI=High-sensitivity cardiac troponin I; sICAM=Soluble intercellular adhesion molecule-1; IL=Interleukin; MPO=Myeloperoxidase; sTREM-1=Soluble triggering receptor expressed on myeloid cells 1; N.S.=non-significant.

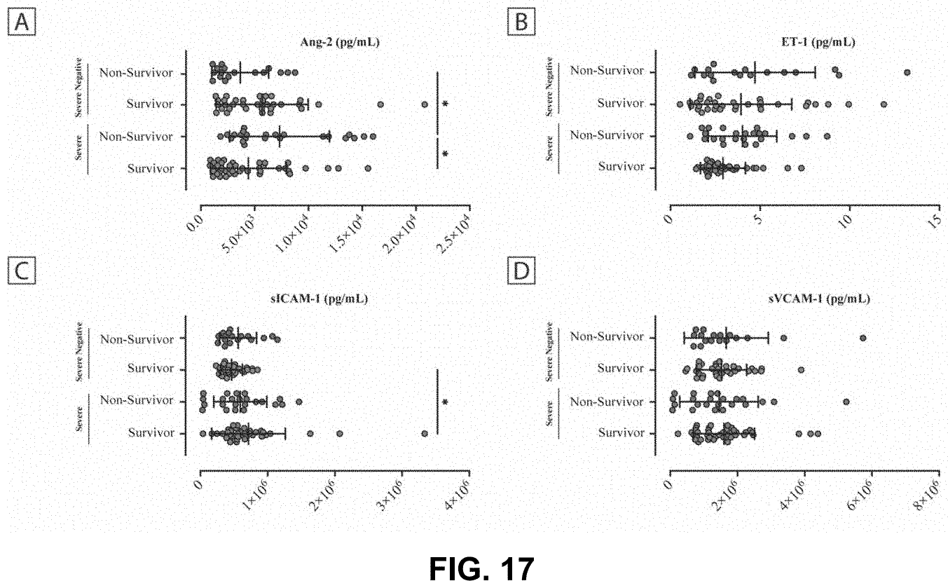

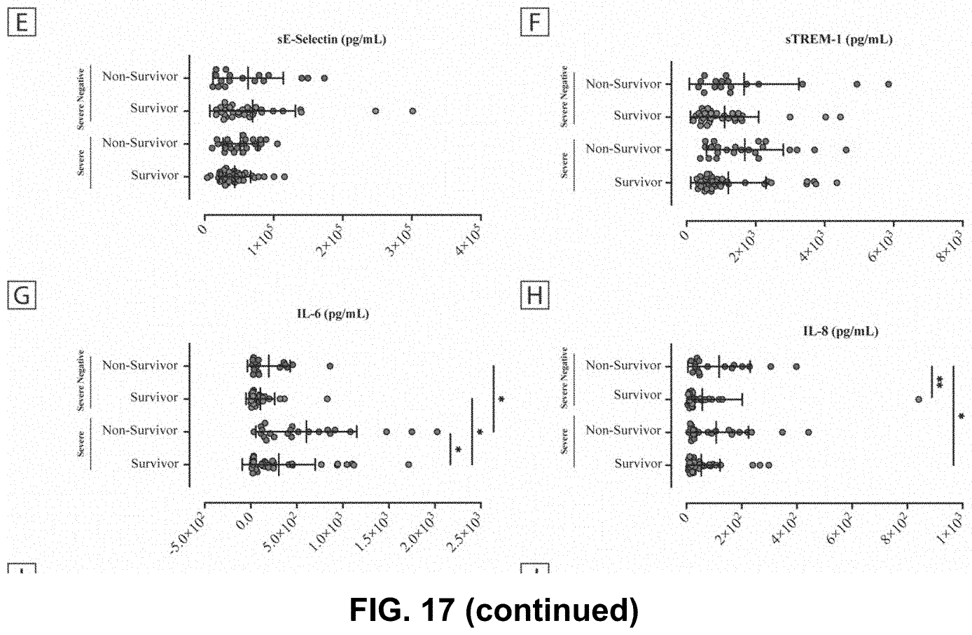

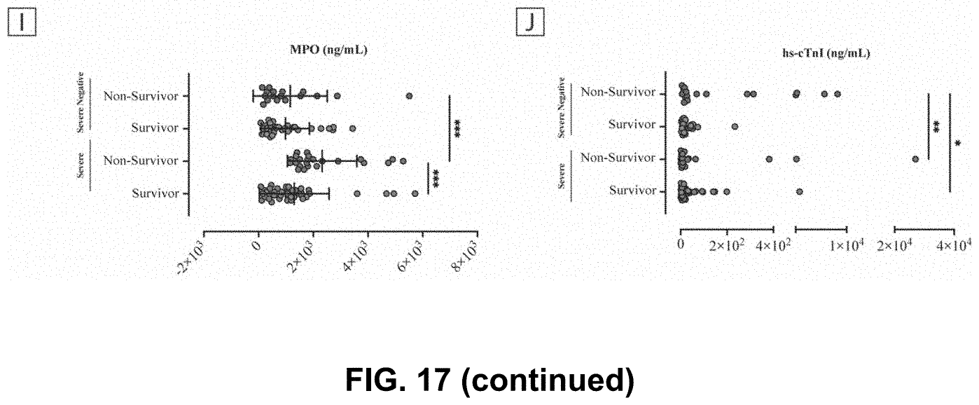

FIG. 17; Related to FIG. 2: Plasma Concentration of Endothelial Dysfunction and Inflammatory Markers at t₀₋₁ and ability to discriminate survival in ICU patients. Severe COVID-19 patients and severe negative patients (i.e., SARS-CoV-2 negative) (a) Levels of Angiopoietin-2, (b) Endothelin-1, (c) sICAM-1, (d) sVCAM-1, (e) sE-Selectin, (f) sTREM-1, (g) IL-6, (h) IL-8, (i) MPO, and (j) hs-cTnI stratified among disease severity. Data shown are for all severe patients with an available t₀₋₁ sample (n=114), with the center bars representing the mean and error bars representing standard deviation (±S.D.). P values for multiple group comparisons were determined by Kruskal-Wallis test with Dunn's multiple comparisons test. Abbreviations: Ang-2=Angiopoietin-2; sICAM=Soluble intercellular adhesion molecule 1; IL=Interleukin; MPO=Myeloperoxidase; sTREM-1=Soluble triggering receptor expressed on myeloid cells 1; sVCAM-1=Soluble vascular cell adhesion molecule 1.

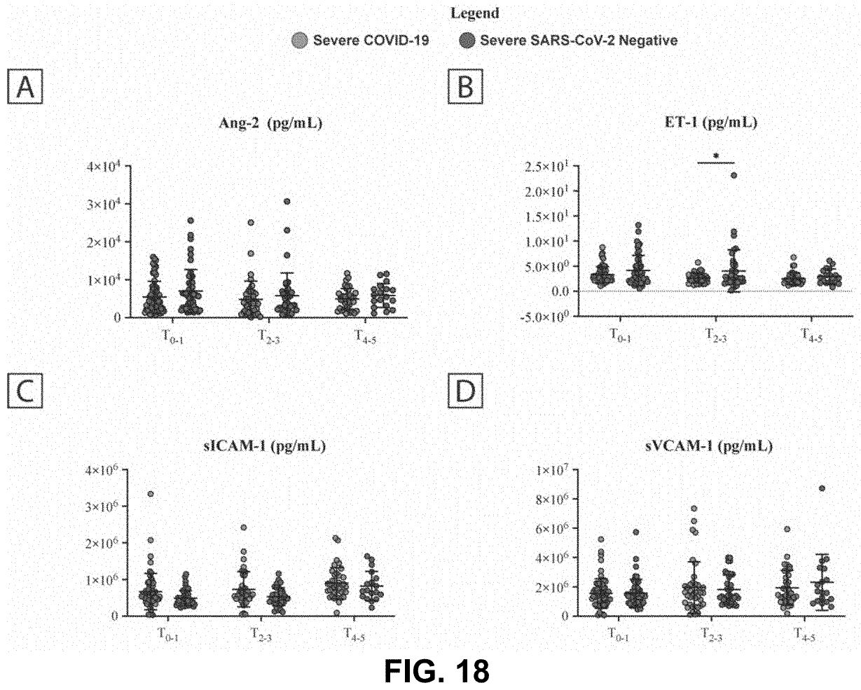

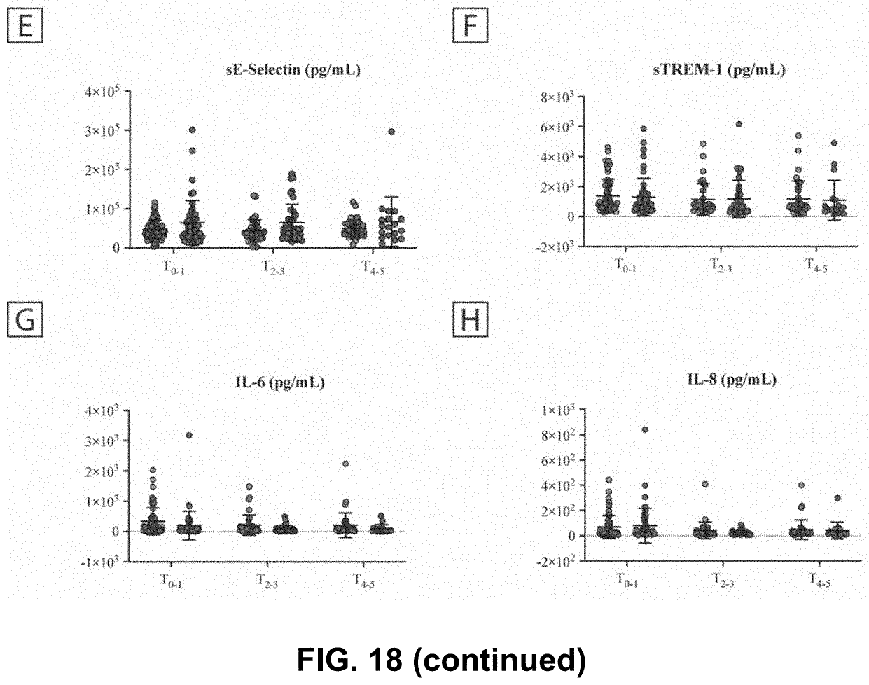

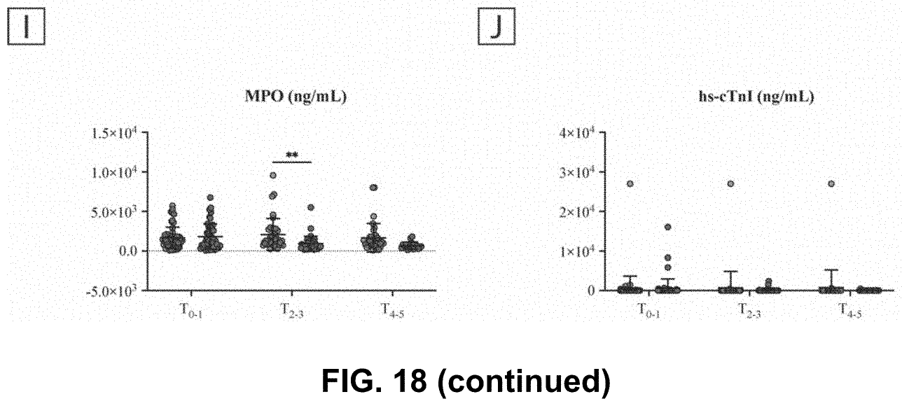

FIG. 18; Related to FIG. 2: Plasma Concentration of Endothelial Dysfunction and Immunological Markers at t₀₋₁ in ICU patients. (a) Levels of Angiopoietin-2, (b) Endothelin-1, (c) sICAM-1, (d) sVCAM-1, (e) sE-Selectin, (f) sTREM-1, (g) IL-6, (h) IL-8, (i) MPO, and (j) hs-cTnI stratified among disease severity. Data shown are for all patients with an available t₀₋₁ (n=114), t₂₋₃ (n=84), and t₄₋₅ (n=44), with center bars representing the mean and error bars representing standard deviation (±S.D.). P values for multiple group comparisons were determined by 2-way ANOVA with Sidak's multiple comparisons test. Abbreviations: sICAM=Soluble intercellular adhesion molecule 1; IL=Interleukin; MPO=Myeloperoxidase; sTREM-1=Soluble triggering receptor expressed on myeloid cells 1; sVCAM-1=Soluble vascular cell adhesion molecule 1.

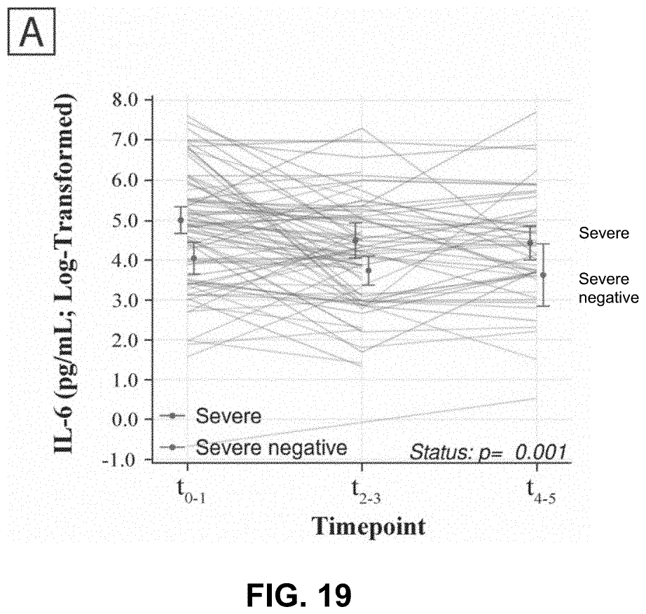

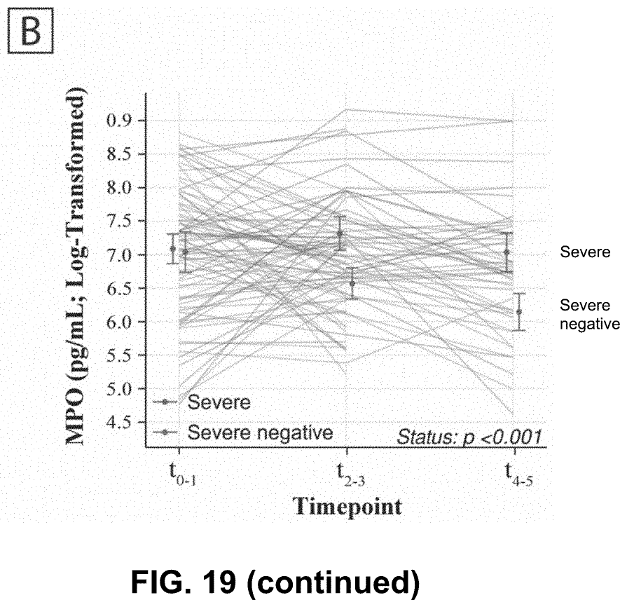

FIG. 19; Related to FIG. 2: Plasma Concentration of (a) IL-6 and (b) MPO, longitudinally between severe COVID-19 patients and severe SARS-CoV-2 negative patients. The error bars represent the mean and its 95% confidence intervals estimated using generalized estimating equation with an independent working correlation matrix. The standard errors were estimated using robust sandwich estimator. Abbreviations: IL=Interleukin; MPO=Myeloperoxidase.

FIG. 20; Related to FIGS. 3 and 4: Plasma MicroRNA Transcriptome Across the Disease Severity Subgroups. Volcano plots of differentially expressed miRNA between patient groups (a, c, e) with predicted KEGG terms (with enrichment score below and number of genes to the right) for pathways of deregulated microRNAs shown beside each corresponding region of the volcano plot (b, d, f). Data is displayed as FDR adjusted P values (Q values) vs the log 2 fold change, with dashed lines are drawn to define restriction boundaries. Data Files containing a full list of differentially expressed miRNA along with a full list of predicted KEGG pathways available (raw data not presented herein). Abbreviations: COVID-19=Coronavirus Disease 2019; FDR=False discovery rate; KEGG=Kyoto Encyclopedia of Genes and Genomes; MiRNAs=MicroRNAs; SARS-CoV-2=Severe acute respiratory syndrome coronavirus.

FIG. 21; Related to FIGS. 3 and 4: Plasma MicroRNA Transcriptome Across the Disease Severity Subgroups. Volcano plots of differentially expressed miRNA between patient groups (a, c, e) with predicted KEGG terms (with enrichment score below and number of genes to the right) for pathways of deregulated microRNAs shown beside each corresponding region of the volcano plot (b, d, f). Data is displayed as FDR adjusted P values (Q values) vs the log 2 fold change, with dashed lines are drawn to define restriction boundaries. Data Files containing a full list of differentially expressed miRNA along with a full list of predicted KEGG pathways available (raw data not presented herein). Abbreviations: COVID-19=Coronavirus Disease 2019; FDR=False discovery rate; KEGG=Kyoto Encyclopedia of Genes and Genomes; MiRNAs=MicroRNAs; SARS-CoV-2=Severe acute respiratory syndrome coronavirus.

FIG. 22; Related to FIG. 4: Feature importance of a machine learning model incorporating clinical data. All clinical metrics are at time of admission with preexisting conditions defined according to those listed in the methods. Abbreviations: BMI=Body mass index; CAD=Coronary Artery Disease; COPD=Chronic obstructive pulmonary disease.

FIG. 23; Related to FIG. 4: Feature importance of a machine learning model incorporating both clinical data and protein expression metrics. All clinical metrics are at time of admission with preexisting conditions defined according to those listed in the methods. Abbreviations: Ang-2=Angiopoietin-2; BMI=Body mass index; CAD=Coronary Artery Disease; ET-1=Endothelin-1; Hs-cTnI=High-sensitivity cardiac troponin I; IL=Interleukin; MPO=Myeloperoxidase; sICAM=Soluble intercellular adhesion molecule-1; sTREM-1=Soluble triggering receptor expressed on myeloid cells-1; sVCAM-1=Soluble vascular cell adhesion molecule-1.

FIG. 24; Related to FIG. 5: Association of Biomarkers with In-Hospital Mortality for Severe COVID-19 Patients. Univariable log hazard ratios of candidate microRNAs (a) hsa-miR-181a-5p, (b) hsa-miR-199a-3p, and c) hsa-miR-339-3p.

FIG. 25; Related to FIG. 5: T₀₋₁ COVID-19 Patient Plasma Selectively Induces Acute Increases in Endothelial Permeability. (a) Permeability of pHUVEC monolayers was measured by 40 kDa FITC extravasation from the apical to the basolateral surface one-hour post-co-incubation. Treatment groups were normalized to the negative control. Center line represents the mean and error bars are (±S.D.). P values determined by one-way ANOVA test with Tukey's multiple comparisons test. Moderate vs negative: **P=9.5×10⁻³; Severe vs negative****P=5.6×10⁻⁸; Severe vs moderate (P): ***P=6.5×10⁻⁴; Severe vs severe negative: ****P=9.8×10⁻⁵. (b) Permeability of pHUVEC monolayers was measured by 40 kDa FITC extravasation from the apical to the basolateral surface six hours post-co-incubation. Treatment groups were normalized to the negative control. Center bars represent the mean and error bars represent the standard deviation (±S.D.). P were values determined by one-way ANOVA test with Tukey's multiple comparisons test. Severe vs negative: *p=2.1×10⁻²; Severe vs severe negative: *P=3.2×10⁻²; n=7-8 per group. Thrombin treatment was included as a barrier disrupting positive control.

FIG. 26; Related to FIG. 5: Correlation of t₀₋₁ Plasma Cardiovascular Biomarkers in COVID-19 positive patients to Induction of Endothelial Permeability. Pearson correlations between (a) hemolysis, (b) Ang-2, (c) hs-cTnI, (d) sE-Selectin, (e) ET-1, (f) sICAM-1, (g) IL-6, (h) IL-8, (i) sTREM-1, (j) sVCAM-1, and (k) MPO to the change in pHUVEC TEER after six-hours co-incubation; n=111 per correlation. Leak is defined through negative values on the x-axis. Abbreviations: Ang-2=Angiopoietin-2; ET-1=Endothelin-1; Hs-cTnI=High-sensitivity cardiac troponin I; IL=Interleukin; MPO=Myeloperoxidase; sICAM=Soluble intercellular adhesion molecule-1; sTREM-1=Soluble triggering receptor expressed on myeloid cells-1; sVCAM-1=Soluble vascular cell adhesion molecule-1.

FIG. 27; Related to FIG. 6: Endogenous sSlit2 is upregulated in severe COVID-19 patient plasma. (a) Endogenous sSlit2 at t₀₋₁ across the severity of COVID-19 (n=27-40, severe vs negative, *P=0.0279). (b) Endogenous sSlit2 at longitudinal intervals in patients with severe COVID-19 (n=14-38). Center bars represent the mean and error bars represent the standard deviation (±S.D.). P values were determined by one-way ANOVA test with Tukey's multiple comparisons test.

### SUPPLEMENTAL REFERENCES

1. Health. Nlo. COVID-19 Treatment Guidelines Panel. Coronavirus Disease 2019 (COVID-19) Treatment Guidelines. 2021 (https://www.covid19treatmentguidelines.nih.gov/.).
2. Ontario PH. COVID-19 Provincial Testing Guidance Update. 2020 (http://www.health.gov.on.ca/en/pro/programs/publichealth/coronavirus/docs/2019_testing_guidance.pdf).
3. Ontario PH. Ontario COVID-19 Data Tool. 2021.
4. Organization WH. (https://www.who.int/news-room/fact-sheets/detail/obesity-and-overweight).
5. Larvin M, Mcmahon M. APACHE-II score for assessment and monitoring of acute pancreatitis. The Lancet 1989; 334(8656):201-205.
6. Ferreira F L, Bota D P, Bross A, Mélot C, Vincent J-L. Serial evaluation of the SOFA score to predict outcome in critically ill patients. Jama 2001; 286(14):1754-1758.
7. Miklas J W, Dallabrida S M, Reis L A, Ismail N, Rupnick M, Radisic M. QHREDGS Enhances Tube Formation, Metabolism and Survival of Endothelial Cells in Collagen-Chitosan Hydrogels. PLOS ONE 2013; 8(8):e72956. DOI: 10.1371/journal.pone.0072956.
8. Sherchan P, Huang L, Akyol O, Reis C, Tang J, Zhang J H. Recombinant Slit2 Reduces Surgical Brain Injury Induced Blood Brain Barrier Disruption via Robo4 Dependent Rac1 Activation in a Rodent Model. Scientific Reports 2017; 7(1):746. DOI: 10.1038/541598-017-00827-z.
9. Francois B, Wittebole X, Ferrer R, et al. Nangibotide in patients with septic shock: a Phase 2a randomized controlled clinical trial. Intensive Care Medicine 2020; 46(7):1425-1437. DOI: 10.1007/s00134-020-06109-z.
10. Murata T, Hori M, Lee S, et al. Vascular endothelium has a local anti-adenovirus vector system and glucocorticoid optimizes its gene transduction. Arteriosclerosis, thrombosis, and vascular biology 2005; 25(9):1796-1803.
11. DiStefano P V, Kuebel J M, Sarelius I H, Glading A J. KRIT1 Protein Depletion Modifies Endothelial Cell Behavior via Increased Vascular Endothelial Growth Factor (VEGF) Signaling. Journal of Biological Chemistry 2014; 289(47):33054-33065. DOI: 10.1074/jbc.M114.582304.
12. Andrews S. FastQC: a quality control tool for high throughput sequence data. Babraham Bioinformatics, Babraham Institute, Cambridge, United Kingdom; 2010.
13. Soneson C, Love M I, Robinson M D. Differential analyses for RNA-seq: transcript-level estimates improve gene-level inferences. F1000Research 2015; 4.
14. Dobin A, Davis C A, Schlesinger F, et al. STAR: ultrafast universal RNA-seq aligner. Bioinformatics 2013; 29(1):15-21. (In eng). DOI: 10.1093/bioinformatics/bts635.
15. Ewels P, Magnusson M, Lundin S, Käller M. MultiQC: summarize analysis results for multiple tools and samples in a single report. Bioinformatics 2016; 32(19):3047-3048.
16. Patro R, Duggal G, Love M I, Irizarry R A, Kingsford C. Salmon provides fast and bias-aware quantification of transcript expression. Nature methods 2017; 14(4):417-419.
17. Love M I, Huber W, Anders S. Moderated estimation of fold change and dispersion for RNA-seq data with DESeq2. Genome biology 2014; 15(12):1-21.
18. Leek J T, Storey J D. Capturing heterogeneity in gene expression studies by surrogate variable analysis. PLoS Genet 2007; 3(9):1724-35. (In eng). DOI: 10.1371/journal.pgen.0030161.
19. Korotkevich G, Sukhov V, Budin N, Shpak B, Artyomov M, Sergushichev A. Fast gene set enrichment analysis. bioRxiv; 2016.
20. Reimand J, Arak T, Adler P, et al. g:Profiler-a web server for functional interpretation of gene lists (2016 update). Nucleic Acids Res 2016; 44 (W1):W83-9. (In eng). DOI: 10.1093/nar/gkw199.
21. Schindelin J, Arganda-Carreras I, Frise E, et al. Fiji: an open-source platform for biological-image analysis. Nature Methods 2012; 9(7):676-682. DOI: 10.1038/nmeth.2019.
22. BioCarta. Biotech Software & Internet Report 2001; 2(3):117-120. DOI: 10.1089/152791601750294344.
23. Kanehisa M, Goto S. KEGG: kyoto encyclopedia of genes and genomes. Nucleic Acids Res 2000; 28(1):27-30. (In eng). DOI: 10.1093/nar/28.1.27.
24. Jassal B, Matthews L, Viteri G, et al. The reactome pathway knowledgebase. Nucleic Acids Res 2020; 48 (D1):D498-d503. (In eng). DOI: 10.1093/nar/gkz1031.
25. Team RC. R: A language and environment for statistical computing. 2020 (https://www.R-project.org/.).
26. Pedregosa F, Varoquaux G, Gramfort A, et al. Scikit-learn: Machine learning in Python. the Journal of machine Learning research 2011; 12:2825-2830.
27. Breiman L. Random forests. Machine learning 2001; 45(1):5-32.
28. Vlachos I S, Zagganas K, Paraskevopoulou M D, et al. DIANA-miRPath v3.0: deciphering microRNA function with experimental support. Nucleic acids research 2015; 43 (W1):W460-W466. (In eng). DOI: 10.1093/nar/gkv403.
29. Okonechnikov K, Conesa A, Garciá-Alcalde F. Qualimap 2: advanced multi-sample quality control for high-throughput sequencing data. Bioinformatics 2016; 32(2):292-294.
30. DeLuca D S, Levin J Z, Sivachenko A, et al. RNA-SeQC: RNA-seq metrics for quality control and process optimization. Bioinformatics 2012; 28(11):1530-1532.

### Additional Embodiments

1. A method of treating Coronavirus Disease 2019 (COVID-19) in a subject or inhibiting the development of COVID-19 or a sequelae resulting from COVID-19 in a subject, the method comprising administering said subject with Slit2, or a variant or fragment thereof.

2. The method of 1 comprising administering said subject with a full-length Slit2.

3. The method of 1, comprising administering said subject with a N-terminal domain of Slit2 (Slit2-N).

4. A method of treating Coronavirus Disease 2019 (COVID-19) in a subject or inhibiting the development of COVID-19 or a sequelae resulting from COVID-19 in a subject, the method comprising upregulating the Slit2 signaling pathway in the subject.

5. The method of 4, comprising administering said subject with one or more factors or polypeptides from the Slit2 signaling pathway.

6. The method of 4, wherein the one or more factors or polypeptides from the Slit2 signaling pathway is GIT1, ARF6, Ab1, GTPase, Ena, Myo9b, or Hakai.

What is claimed is:

1. A method of determining Coronavirus Disease 2019 (COVID-19) severity in a subject, the method comprising:
obtaining a circulating blood sample from the subject;
obtaining a biomarker panel comprising one or more of angiopoietin-2 (Ang-2), endothelin-1 (ET-1), soluble intercellular adhesion molecule (sICAM), soluble vascular cell adhesion molecule (sVCAM), soluble E-selectin (sE-selectin), triggering receptor expressed on myeloid cells-1 (sTREM-1), interkeulin-6 (IL-6), interleukin-8 (IL-8), myeloperoxidase (MPO), and high-sensitivity cardiac troponin (hs-cTnI);
wherein a different level of at least one biomarker in the biomarker panel compared to a reference panel from a SARS-CoV-2-negative subject is indicative of COVID-19 severity.

2. The method of claim 1, wherein the biomarker panel comprises Ang-2.

3. The method of claim 1, wherein the biomarker panel comprises Ang-2, and MPO.

4. The method of claim 1, wherein the biomarker panel comprises Ang-2, ET-1, sICAM-1, sVCAM-1, sE-Selectin, sTREM-1, IL-6, IL-8, MPO and hs-cTnI.

5. The method of claim 1, wherein an elevated level of at least one biomarker is indicative of COVID-19 severity.

6. A method of determining mortality risk of a subject infected with SARS-CoV-2, the method comprising detecting in a circulating blood sample from the subject one or more circulating microRNA (miR) biomarkers selected from the group consisting of: one or more miR from miR-30 family, miR-181a-5p, miR-199a-3p, miR-4793-5p, miR-6080, and miR-6750-5p.

7. The method of claim 6, wherein the one or more biomarkers are selected from the group consisting of: miR-30b, miR-30c, miR-30e, miR-181a-5p, miR-199a-3p, and miR-6080.

8. The method of claim 6, comprising amplifying and detecting a target miR in the blood sample using polymerase chain reaction (PCR).

9. The method of claim 6, comprising capturing and sequencing the detected miR.

10. The method of claim 9, comprising sequencing the detected miR using next-generation sequencing.

11. The method of claim 9, wherein sequencing the detected miR comprises a nuclease protection assay.

12. The method of claim 6, further comprising measuring the expression level of at least one of the one or more circulating miR biomarkers.

13. The method of claim 12, comprising measuring the expression level of two or more of the circulating miR biomarkers.

14. The method of claim 12, comprising measuring the miR expression level using a multiplex assay, preferably a 5-plex assay.

15. The method of claim 6, wherein detecting the one or more circulating miR biomarkers comprises detecting miR using a colorimetic or a CRISPR-based biosensor.

16. A kit comprising one or more antibodies specific to angiopoietin-2 (Ang-2), endothelin-1 (ET-1), soluble intercellular adhesion molecule (sICAM), soluble vascular cell adhesion molecule (sVCAM), soluble E-selectin (sE-selectin), triggering receptor expressed on myeloid cells-1 (sTREM-1), interkeulin-6 (IL-6), IL-8, myeloperoxidase (MPO), or high-sensitivity cardiac troponin (hs-cTnI).

17. The kit of claim 16, comprising an antibody specific to Ang-2.

18. The kit of claim 16, comprising a first antibody specific to Ang-2, and a second antibody specific to MPO.

19. The kit of claim 18, further comprising one or more antibodies specific to a biomarker selected from Ang-2, ET-1, sICAM-1, sVCAM-1, sE-Selectin, sTREM-1, IL-6, IL-8, and hs-cTnI.

20. A kit comprising one or more probes for binding a circulating microRNA (miR) biomarker selected from the group consisting of: one or more miR from miR-30 family, miR-181a-5p, miR-199a-3p, miR-4793-5p, miR-6080, and miR-6750-5p.

21. The kit of claim 20, wherein the biomarker is selected from the group consisting of: miR-30b, miR-30c, miR-30e, miR-181a-5p, miR-199a-3p, and miR-6080.

22. The kit of claim 16, wherein the kit is for an assay.

23. The kit of claim 20, wherein the one or more probes are amplification probes.

24. The kit of claim 20, wherein the one or more probes are sequencing probes.
